(19) United States
(12) Patent Application Publication
(10) Pub. No.: US 2018/0228866 A1
(43) Pub. Date: Aug. 16, 2018

(54) COMPOSITIONS

(71) Applicant: Sigmoid Pharma Limited, Dublin (IE)

(72) Inventor: Ivan Coulter, Dublin (IE)

(73) Assignee: Sigmoid Pharma Limited, Dublin (IE)

(21) Appl. No.: 15/751,117

(22) PCT Filed: Aug. 12, 2016
(86) PCT No.: PCT/EP2016/069290

§ 371 Date: Feb. 7, 2018

(30) Foreign Application Priority Data
Aug. 12, 2015 (GB) 1514328.2

(51) Int. Cl. A61K 38/13; A61K 9/16; A61K 9/107; A61P 37/06

(52) U.S. Cl. CPC A61K 38/13; A61K 9/167; A61P 37/06; A61K 9/1617; A61K 9/107; A61K 9/1658

(57) ABSTRACT

The present invention relates to a formulation comprising an inhibitor of NFAT activation for use in treating or preventing undesirable effects, more particularly undesirable effects occurring in conjunction with T cell-mediated therapies. The undesirable effects may be cytokine release syndrome (CRS) or symptoms associated with gastrointestinal (GI) inflammation, for example associated with inflammatory bowel diseases, such as ulcerative colitis, optionally caused by activated T cell activity. In addition to ameliorating undesirable effects, the invention is aimed at also maintaining the therapeutic effects of the T-cell mediated therapy.

This invention relates to a composition comprising a pharmaceutically active ingredient for use in treating, e.g. ameliorating undesirable effects of a therapy, more particularly treating one or more undesirable effects occurring in conjunction with T cell-mediated therapies. The undesirable effects may be cytokine release syndrome (CRS) or symptoms associated with gastrointestinal (GI) inflammation, for example associated with inflammatory bowel diseases, such as ulcerative colitis, optionally caused by activated T cell activity. In addition to ameliorating undesirable effects, the invention is aimed at also maintaining the effectiveness of the therapy. The composition may be applied as an adjunct therapy where undesirable effects are observed or as a prophylactic therapy.

### BACKGROUND

It has long been known that the activity of T cells of the immune system is dependent on antigen-binding to the T cell receptor (TCR) but is regulated by complex interplay of a variety of signalling pathways, both stimulatory and inhibitory. Antigen-specific T cell activation requires two signals between T cells and antigen-presenting cells (APCs). The first signal requires presentation of an antigen to the TCR in conjunction with a major histocompatabilty complex (MHC) molecule on APCs. To complete T cell activation, the interaction of the CD28 receptor on T cells with co-stimulatory molecules on APCs is also required. Negative regulators of T cell immunity (also referred to as checkpoint proteins), including CTLA-4 and the PD-1 checkpoint receptor, are important in healthy individuals for control of T cell activity and preventing inappropriate T cell responses. After stimulation of naïve T cells through the TCR, CTLA-4 is upregulated and competes with CD28. The PD-1 checkpoint protein is a co-inhibitory molecule expressed on chronically stimulated T cells and normally plays a crucial role in modulating T cell activity through interaction with its ligands, PD-L1 and PD-L2. PD-L1, and to a lesser extent PD-L2, have been found to be expressed on many hematologic and non-hematologic human tumours. Immune checkpoint blockade by antibody targeting of for example CTLA-4 and/or PD-1 ligand binding is now recognised as one of a number of immunologically-based strategies for tackling various forms of cancer which harnesses the activity of autologous T cells (Gelao et al. Toxins (2014) 6, 914-933).

A key protein linking antigen-binding at a TCR with activation of T cell effector function is NFAT (nuclear factor of activated T cells). NFAT1-4 are a family of calcium/calcineurin-regulated transcription factors. Their expression and activation varies among different cell types and their function depends on cellular context. Increase in cytoplasmic Ca²⁺ upon antigen-TCR binding increases the activity of calcineurin which dephosphoryates NFAT. When dephosphorylated, NFAT translocates to the nucleus where it interacts with other transcription factors to drive the expression of various pro-inflammatory cytokines. In the nucleus NFAT is known for example to interact with AP-1. Together, the pair bind to regulatory regions in the genome and initiate a genetic program that activates T cells. Recently, evidence has been presented that depending on the availability of AP-1, NFAT tips the scale toward T cell activation or exhaustion; without it, NFAT initiates a negative regulatory program (Martinez et al. (February 2015) Immunity 42, 265-278). PD-1/PD-L1 binding is known to affect NFAT activation of T cells. Hence, blockage of PD-1:PD-L1 interaction with either anti-PD-1 or anti PD-L1 antibodies can reactivate the NFAT activation pathway (Cong et al. (May 2015) Gen. Eng. Biotechnol. News vol 35, No. 10). NFAT also interacts with transcription coactivators such as CREB-binding protein (CBP) and p300.

While calcineurin, which promotes the nuclear entry of NFAT, is the most well characterized signalling molecule in NFAT activation, it is not alone. A number of other T cell signalling molecules have also been linked to NFAT activation. Ras and protein kinase C stimulate the synthesis and activation of Jun/Fos for the full activation of the NFAT-AP-1 complex. c-Raf and Rac have been shown to promote a NFAT-CBP interaction. In contrast, the phosphorylation of NFAT by glycogen synthase kinase 3 leads to the nuclear export of NFAT. By blocking NFAT activation, glycogen synthase kinase 3 has been shown to be a negative regulator of T cell activation. Among different MAPKs, c-Jun N-terminal kinase inhibits the targeting of calcineurin to NFAT in T cells, and extracellular signal-regulated kinase increases the nuclear export of NFAT. It has been shown that p38 MAPK phosphorylates NFATp and NFAT3 to promote their nuclear export. p38 MAPK activates the NFAT promoter, stabilizes NFAT mRNA, increases NFAT translation, and promotes NFAT-CBP binding. The overall effect of p38 MAPK is the activation of NFAT.

It has been shown that, unlike resting Tconv cells where NFAT is exported from the nucleus and resides in the cytoplasm in its inactive phosphorylated form, a fraction of NFAT protein constitutively localizes in the nucleus of primary Tregs, where, through interacting with various transcription factors, it selectively binds to Foxp3 target genes. Therefore, when Treg cells are exposed to calcinueurin inhibitors, export of NFAT from the nucleus is not induced, thus indicating that its nuclear translocation is independent of calcineurin activity. Importantly, while Tregs are resistant to calcinueurin inhibitors in the presence of IL-2 and continue to proliferate in response to anti-CD3 stimulation, proliferation of non-Tregs is abrogated by calcinueurin inhibitors. In addition, PMA, which activates other transcription factors required for T cell activation but not NFAT, selectively induces Treg proliferation in the absence of ionomycin. TCR interaction with self-MHC class II is not required for PMA-induced Treg proliferation. Uniquely, Tregs expanded by PMA or in the presence of calcinueurin inhibitors maintain Treg phenotype and functionality (Li, Q et al., Constitutive Nuclear Localization of NFAT in Foxp3+ Regulatory T Cells Independent of Calcineurin Activity J Immunol. 2012 May 1; 188(9):4268-77).

The role of NFAT in controlling regulatory T cells—thymus-derived naturally occurring regulatory T cells (nTreg)—similar to that in T cony cells involves calcium signals. However, unlike in Tconv cells, Treg control and activity involves interaction of NFAT with different transcription factors such as Foxp3 (Fork head box P3) that result in binding to NFAT binding regions of the Foxp3 gene. The generation of peripherally induced Treg (iTreg) by TGF-β is known to be dependent on NFAT expression because the ability of CD4+ T cells to differentiate into iTreg diminished markedly with the number of NFAT family members missing. It can be concluded that the expression of Foxp3 in TGF-β-induced iTreg depends on the threshold value of NFAT rather than on an individual member present. This is specific for iTreg development, because frequency of nTreg remained unaltered in mice lacking NFAT1, NFAT2, or NFAT4 alone or in combination. Importantly, the function of both nTreg and iTreg was independent on robust NFAT levels, reflected by less nuclear NFAT in nTreg and iTreg. It has previously been shown that Treg cells can suppress colitis or transplant rejection in mouse models. Vaeth demonstrated that deletion of NFAT members did not alter suppressor activity in vitro or during colitis and transplantation in vivo. This scenario emphasizes an inhibition of high NFAT activity as treatment for autoimmune diseases and in transplantation, selectively targeting the proinflammatory conventional T cells, while keeping Treg functional (Vaeth M, et al. (2012). Dependence on nuclear factor of activated T cells (NFAT) level discriminates conventional T cells from Foxp3+ regulatory T cells. Proc Natl Acad Sci USA 109(40):16258-16263).

It has recently been established that graft-versus-host disease (GvHD), an immunological complication of allogeneic hematopoietic stem cell transplantation (allo-HCT), is driven largely by NFAT-1 and NFAT-2 activated T cells. An important factor which promotes GvHD is the necessary conditioning regime for allo-HCT employing chemo- and/or radio-therapy. Such conditioning is liable to be accompanied by GI barrier damage and local release of inflammatory cytokines. This promotes T cell activation through NFAT-activation with consequent pro-inflammatory cytokine secretion and may result in a cytokine storm. In contrast, it has recently been established through selective inhibition or deletion of Foxp3+ regulatory T cells, also provided amongst donor T cells by allo-HCT treatment, that Treg cells lacking NFAT-1 and NFAT-2 retain activity. Also, the ability of Tconv cells deficient in NFAT-1 and NFAT-2 to induce GvHD is limited. Therefore, it is suggested that NFAT activity in Tregs, a population of cells that are believed to be important in promoting the desirable graft-versus leukaemia (GvL) effect of the transplantation, is not essential. It is noteworthy that, compared to normal NFAT expressing T cells, NFAT-deficient T cell propagation and homing to target cells, tissues or organs is reduced. (Vaeth et al. (January 2015) PNAS 112, 1125-1130).

NFAT activation plays an important role in T and B cell activation and T and B cell development as well as a range of other immune cells, including dendritic cells. The spatial-temporal activation patterns of proliferating T lymphocytes during graft-versus-host disease (GVHD) and T cell precursors during T cell development after allogeneic hematopoietic stem cell transplantation (HSCT) showed that in the first days after HSCT, donor T cells migrated to the peripheral lymph nodes and the intestines, with NFAT activation dominant in the intestines, suggesting an important role for the NFAT activated donor T cells in the intestines in the early stages of alloactivation during development of GVHD. The activation of NFAT following T Cell Receptor stimulation, involving dephosphorylation of NFAT proteins and subsequent translocation of the dephosphorylated NFAT from the cytoplasm to the nucleus, is rapid and is implicated in the regulation of several important genes associated with T cell activation, including, but not limited to, interleukin-2 (IL-2), interferonγ (IFNγ), tumour necrosis factor-α (TNFα). In the first 2 to 3 days after BMT, donor T cells migrated to the peripheral lymph nodes (PLN) and the intestines, but NFAT activation was predominantly seen in the intestines, and not in the PLN. In the following days, 4 to 8, the NFAT activity was comparable; however, there were significantly more T cells in the PLN than in the gut, resulting in a higher percentage of activated T cells overall in the gut. Subsequently, the NFAT activation to constitutive signal ratio was higher in the gut at each time point (Na et al. Concurrent visualization of trafficking, expansion, and activation of T lymphocytes and T cell precursors in vivo; Blood, 16 Sep. 2010 volume 116, no. 11). This observation suggests either more activation per cell or a relatively higher percentage of activated cells in the gut. All indicate a dominant role for the intestines in the early stages of alloactivation during the development of GvHD.

Conventional treatment for GvHD employs NFAT inhibitors such as cyclosporin A or tacrolimus (FK506). Currently, the only available formulations of each drug are intended to provide broad systemic exposure following administration by injection or per orally. Both these drugs inhibit calcineurin action and thereby suppress activation of NFAT. At the doses required to provide benefit in GvHD, generally high doses, first administered by injection followed by initial higher doses administered per orally, the entire body is exposed to high levels of broad NFAT suppression, a suppression that does not discriminate between various members of the NFAT family. At such high doses, the systemic concentrations are known to have significant potential to cause severe adverse events directly to tissues and cells in organs such as the kidney, liver, central nervous system and cardiovascular system. In addition, broad systemic NFAT inhibition results in suppression of IL-2 expression throughout the body. So, NFAT inhibitors such as cyclosporine A and tacrolimus, in addition to the known potential to cause severe adverse effects may also interfere with the GvL effect through indirect perturbation of Treg function due to impaired IL-2 production of effector T cells as well as through reduced propagation of donated T cells. Investigation of the spatial-temporal activation pattern of NFAT activation during acute GvHD in mouse models revealed that the strongest NFAT activation is in the gastrointestinal tract. Furthermore, it is recognised that NFAT activity is linked to upregulation of the gut-homing receptor α4β7 integrin. Compositions for oral delivery of CsA as disclosed in WO 2008/122967 (in common ownership with the present application) are designed to improve the benefit versus adverse effect balance of CsA use for GvHD treatment.

In allo-HCT treatment, NFAT-activated T cells are solely the source of a potential life threatening problem, a problem that can be reduced when NFAT expression is reduced or eliminated. The beneficial GvL effects associated with allo-HCT Tcells, including those associated with Tregs, is not affected to a lesser extent by NFAT inhibition. In addition to immunomodulation through the traditional allo-HCT cell therapy, it is also now recognised that clinical immunotherapy through the provision or promotion of NFAT-activated T cells can provide an important therapeutic strategy in the fight against various diseases, especially cancers as well as infection, including chronic infections such as HIV-1/AIDS. Immune checkpoint blockade as already referred to above, leading to promotion of autologous T cell attack on tumours, is just one of a number of approaches exemplifying such immune strategy mediated by NFAT-activated T cells. Other such approaches now receiving much interest are T cell engaging therapies wherein T cells are activated when directly or indirectly bound to target disease antigen, e.g. a tumour antigen, via a receptor at the surface of the T cells. Such therapies include use of multivalent, including bi-specific T cell engagers, including bispecific antibodies and bispecific constructs in which an engineered TCR is linked to an anti-CD3 antibody fragment (ScFv) which engages T cells (exemplified by ImmTACs (immune mobilising monoclonal TCRs against cancer) as under investigation by immunocore Limited). They also include use of otherwise modified T cells, including Treg cells, engineered to present a chimeric antigen receptor, CAR-T therapy as well as TCR-engineered T-cells. Such cells may be autologous cells engineered ex vivo and returned to the same patient. However, engineered allogenic CAR-T cells are also under investigation wherein the normal TCR is subject to inactivation by gene editing. While such therapies have immense therapeutic potential, they are not without undesirable side effects, which can prove life threatening in some instances and are the innate consequence of NFAT activation in effector T cells.

Just as excessive release of pro-inflammatory cytokines is a feature associated with activated T cells mediating GvHD, such a cytokine storm is liable to occur with cell therapies mediated by NFAT-activated T cells and is generally referred to as cytokine release syndrome. CRS is characterised by fever, nausea, headache, tachycardia, hypotension, rash and shortness of breath and may also have neurologic manifestations. CRS occurrence is not rare but is often manageable. However, there have been fatalities in subjects administered with a T cell-based therapy who have subsequently developed CRS. CRS has for example been of particular note in recent clinical trials of autologous CAR-T therapy to treat haematological malignancies as reviewed in Xu et al. (2014) Cancer Letters 343, 172-178: ‘Cytokine release syndrome in cancer immunotherapy with chimeric antigen receptor engineered T cells.’ See also Maude et al. (2014) Cancer J. 20, 119-122: ‘Managing Cytokine Release Syndrome Associated with Novel T Cell-engaging Therapies’ and Minagawa et al. (May 2015) Pharmaceuticals 8, 230-249: ‘Seatbelts in CAR therapy: How safe are CARs?’

The liability for, and seriousness of, CRS development with such T cell therapy has been linked to pre-conditioning as well as baseline cytokine levels, the tumour burden and the T cell dosing. When the baseline cytokine level is high at the time of CAR-T cell infusion or a there is a large amount of CAR-T cells encountering with target cells over a short time, CRS can be triggered quickly and severely. While the cytokine profiles vary greatly, IFN-γ, TNF-α and IL-6 are the most frequently monitored cytokines. IFN-γ and IL-6 are increased more than 10-fold in most patients with problematic CRS.

The administration of biologics resulting in cytokine release syndrome was first reported to be associated with the administration of anti-CD3 mAb OTK3, a biologic that is known to induce NFAT, which was administered as a systemic immunosuppressive agent during organ transplantation. Within 1-4 hours after OKT3 injection, serum levels of proinflammatory cytokines such as TNF-α, IFN-γ, and IL-6 were markedly elevated. Most recently a cytokine storm was reported in six of six patients that were treated with anti-CD28 mAb TGN1412. In that report, TNF-α levels peaked within 1 hour after infusion and IL-2, IL-6, IL-10, and IFN-γ reached maximum levels at the next time point, 4 hours after infusion (elevation in other cytokines included IL-4, IL-8, IL-12, and IL-1β). All six patients in that study required supportive care in an intensive care unit and two of the six required extensive intensive care unit stays of 11 and 21 days.

Treatment of CRS has been reported with corticosteroids and more recently with the anti-IL-6 monoclonal antibody tocilizumab. Corticosteroids treat CRS but also halt the therapeutic activity of the cell therapy. On the other hand tocilizumab has been reported, but not fully validated, as treating CRS with retention of therapeutic effect. Also, while tocilizumab suggests the benefit of using anti-IL-6 antibodies in treating CRS, it is not clear if it could be equally effectively employed as an approach to prevent CRS and act as an adjunct therapy to the various T cell modulating therapeutic approaches outlined above. Also, as injectable monoclonals, the IL-6 antibodies may be prohibitively expensive. Therefore, there is a need for a treatment that treats, reduces, and/or prevents CRS whilst maintaining therapeutic activity of the associated T cell mediated therapy.

Symptoms associated with inflammatory bowel disease have been noted as an undesirable “immune-related adverse event” associated with T cell mediated therapy, including immune check point blockade (Gelao et al. ibid). This is consistent with the fact that in both ulcerative colitis (UC) and Crohn's disease, the inflamed tissue is heavily infiltrated with activated T cells secreting large amounts of cytokines. Shih et al. reported nuclear translocation and activation of NFAT2 in infiltrating lymphocytes of UC diseased colonic mucosa (World J. Gasteroenterol. 14, 1759-1767).

Cytokine elevations are measurable in most patients, but the degree of elevation may not correlate with severity of CRS or response to therapy. Moreover, some patients experience symptoms without marked cytokine elevation, whereas others demonstrate laboratory findings out of proportion to clinical symptoms.

Consequently, it can be difficult to identify a subject with CRS that may resolve or a subject with CRS that might be fatal. Therefore, there is a need for a clinically effective, cost effective therapy that can be administered as an adjunct therapy throughout a T cell mediated therapy.

Cyclosporin A is a cyclic polypeptide which has immunosuppressive and anti-inflammatory properties. The compound has been approved for the prevention of organ rejection following kidney, liver, heart, combined heart-lung, lung or pancreas transplantation, for the prevention of rejection following bone marrow transplantation; the treatment and prophylaxis of Graft Versus Host Disease (GVHD); psoriasis; atopic dermatitis, rheumatoid arthritis and nephrotic syndrome (Neoral™ Summary of Product Characteristics 24/02/2012). Cyclosporin A may also be useful for the treatment of a range of other diseases including for the treatment of severe recalcitrant plaque psoriasis Bechet's disease, anaemia, myasthenia gravis and various conditions affecting the GI tract, including irritable bowel syndrome, Crohn's disease, colitis, including ulcerative colitis, diverticulitis, pouchitis, proctitis, Gastro-Intestinal Graft Versus Host Disease (GI-GVHD), colorectal carcinoma and adenocarcinoma as well as ischemia induced disease. A range of other diseases may benefit from treatment with cyclosporin A (Landford et al. (1998) Ann Intern Med; 128: 1021-1028) the entirety of which is incorporated herein by reference. Cyclosporin A has been used to treat a number of gastrointestinal conditions including inflammatory bowel disease (Sandborn W J, a critical review of cyclosporin therapy in inflammatory bowel disease, Inflamm Bowel Dis. 1995; 1:48-63), including ulcerative colitis (Lichtiger et al, preliminary report (cyclosporine in the treatment of severe ulcerative colitis), Lancet. 1990; 336:16-19; Cohen et al, Intravenous cyclosporine in ulcerative colitis (a five-year experience), Am J Gastroenterol. 1999; 94:1587-1592).

However, as noted above, cyclosporin A has a number of undesirable side effects including hypertension, impaired renal function, and neurotoxicity (Feutren et al, Risk factors for cyclosporine-induced nephropathy in patients with auto-immune diseases, International kidney biopsy registry of cyclosporine for autoimmune diseases, N Engl J Med. 1992; 326:1654-1660; Wijdicks et al., Neurotoxicity in liver transplant recipients with cyclosporine immunosuppression, Neurology. 1995; 45:1962-1964; and Porter et al, Cyclosporine-associated hypertension, National High Blood Pressure Education Program. Arch Intern Med. 1990; 150:280-283).

Cyclosporin A is available as an intravenous formulation; Sandimmun™ which is a solution of 50 mg/ml of cyclosporin A in ethanol and polyethoxylated castor oil (for example Kolliphor™ EL). The product is also available as orally administered formulations, including a soft gelatin capsule containing a solution of cyclosporin A in ethanol, corn oil and lineoyl macrogolglycerides (Sandimmune™ Soft Gelatin capsules) and as an orally administered solution containing the cyclosporin dissolved in olive oil, ethanol, and labrafil M 1944 CS (polyethoxylated oleic glycerides) (Sandimmune™ Oral Solution). More recently a microemulsion concentrate formulation has been approved containing cyclosporin A dissolved in DL-α-tocopherol, absolute ethanol, propylene glycol, corn oil-mono-di-triglycerides, polyoxyl 40 hydrogenated castor oil (Neoral™). Following oral administration the Neoral™ formulation results in the formation of a microemulsion and is stated to have an improved bioavailability compared to orally administered Sandimmune™. These orally administered cyclosporin A compositions, primarily developed with the intent of enabling the systemic immunosuppression believed to be required to prevent solid organ rejection or systemic autoimmune diseases such as rheumatoid arthritis or psoriasis, are all instant release compositions and cyclosporin A will be present at high concentration in the stomach and small intestine from where it is systemically absorbed.

Sandborn et al. (J Clin Pharmacol. 1991; 31:76-80) determined the relative systemic absorption of cyclosporin following oral and intravenous as well as oil- and water-based enemas. Based on negligible plasma cyclosporin concentrations observed following enema administration, it was suggested that cyclosporin, even when solubilised, is poorly absorbed from the colon. The enemas however demonstrated considerable efficacy in the treatment of inflammatory bowel disease (Ranzi T, et al, Lancet 1989; 2:97). Intravenous or orally administered cyclosporin efficacy in the treatment of inflammatory bowel disease is dose dependent, requiring high doses to ensure adequate concentration reaches the colon. Systemic toxicity is known to be dose and duration dependent. At the concentrations required following oral or injected administration of the approved soft-gel or emulsion-based formulations to treat inflammatory bowel disease, the risk of developing side effects is high. Thus, while cyclosporine is noted as a therapeutic option in a number of learned treatment guidelines, its recommended use is limited to no more than 3 months and requires frequent monitoring of drug levels in the blood as well as kidney and liver function, not to mention blood pressure monitoring.

Formulating pharmaceutically active ingredients into a form suitable for administration to a patient is a developed area of science. It is also a key consideration for the efficacy of a drug. There are many examples of methods for formulating drugs and other active ingredients. The aim of these formulations are varied and can range from increasing systemic absorption, allowing for a new route of administration, improving bioavailability, reducing metabolism of the active, or avoiding undesirable routes of administration.

WO 2008/122965 discloses oral cyclosporin minicapsule compositions with modified release properties which release cyclosporin in at least the colon. WO2010/133609 discloses compositions comprising a water-soluble polymer matrix in which are dispersed droplets of oil, the compositions comprising a modified release coating. The disclosed compositions also contain an active principle.

### BRIEF SUMMARY OF THE DISCLOSURE

A novel use of an NFAT activation inhibitor delivered to the GI tract is now proposed to reduce or prevent immune-related adverse effects associated with therapies mediated by NFAT-activated T cells while maintaining effectiveness of the therapy.

There is provided a composition comprising an inhibitor of NFAT activation for use in treating one or more undesirable effects occurring in conjunction with a therapy mediated by NFAT-activated T cells, wherein said undesirable effects are selected from Cytokine Release Syndrome (CRS) and symptoms associated with gastrointestinal inflammation and wherein said composition is administered to the gastrointestinal tract whereby said one or more undesirable effects are treated with maintenance of effectiveness of the therapy.

A composition comprising an inhibitor of NFAT activation may be used to treat one or more undesirable effects occurring in conjunction with a therapy mediated by NFAT-activated T cells. The undesirable effects may be selected from cytokine release syndrome and symptoms associated with gastrointestinal inflammation, e.g. symptoms associated with inflammatory bowel diseases such as ulcerative colitis and Crohn's disease. The composition may be administered to the gastrointestinal tract (GIT). The one or more undesirable effects are treated with maintenance of effectiveness of therapy mediated by NFAT-activated T cells. The composition may be for supplying a NFAT inhibitor to specific regions of the GIT or throughout the entire GIT. The composition may optionally provide for modulated or limited systemic absorption. Throughout the present application the inhibitor of NFAT activation may be referred to as an NFAT inhibitor. The two terms, “inhibitor of NFAT activation” and “NFAT inhibitor”, are therefore considered to be equivalent.

The undesirable effects treated by the composition comprising an inhibitor of NFAT activation occur in conjunction with a therapy mediated by NFAT-activated T cells. This includes when the undesirable effects are caused by the therapy mediated by NFAT-activated T cells, the therapy optionally being co-administered with the composition comprising an inhibitor of NFAT activation. Co-administration covers the situation where the composition comprising an inhibitor of NFAT activation is administered simultaneously, sequentially or separately to the therapy mediated by NFAT-activated T cells.

Furthermore, the composition comprising an inhibitor of NFAT activation may be for use in treating a patient suffering from one or more undesirable effects occurring in conjunction with or caused by the therapy mediated by NFAT-activated T cells.

In embodiments the undesirable effects are NFAT-activated T-cell induced CRS or NFAT-activated T-cell induced GI inflamation occurring in conjunction with or caused by a therapy mediated by NFAT-activated T cells.

Such administration is preferably oral administration employing a controlled-release formulation of an inhibitor of NFAT activation. The composition may be a controlled or modified release composition which releases the inhibitor of NFAT activation to specific sites of the GIT. For example, the composition may be adapted to release the NFAT inhibitor in the stomach, small intestine (duodenum, jejunum or ileum), large intestine (cecum, colon, or rectum) or a combination thereof. Preferably the composition employed is a controlled release formulation of cyclosporin A for oral administration, including known formulations and formulations further discussed below. This provides a cost effective manner of treating undesirable effects, e.g. ameliorating or preventing immune adverse events of concern associated with the therapy mediated by NFAT-activated T cells. As such, intervention need not await any serious effect arising following occurrence of the undesirable effects associated with the therapy.

It will be recognised that a composition of the invention may be administered to treat (ameliorate) a diagnosed undesirable effect, such as an immune adverse event (e.g. CRS) or as a prophylactic composition to prevent or reduce development of such an undesirable effect. Such prophylactic administration may be as an adjunct therapy in conjunction with the T cell mediated therapeutic effect, but may also additionally precede the T cell therapeutic action, e.g. during or after a pre-conditioning regime. Preferably, the composition may be administered to the GI tract throughout any pre-conditioning regime and as an adjunct treatment with the therapy mediated by NFAT-activated T cells.

There are a growing number of therapies which rely on provision or promotion of NFAT-activated T cells to target disease antigens, especially tumour antigens. The invention may find use whenever such activated T cells are employed for intended therapeutic benefit. It is envisaged, however, that its use may be particularly favoured where a pre-conditioning regime, employing chemotherapy and/or radiotherapy, is employed to deplete autologous T cells. In such instances, it may be preferred to administer a composition in accordance with the invention throughout the pre-conditioning regime or at least ahead of the therapy mediated by NFAT-activated T cells. As indicated above, such administration may also desirably continue during the actual therapy period and in instances where the therapy is directed against a haematological disorder may be continued during application of allo-HCT. Examples of therapies mediated by NFAT-activated T cells for which application of the invention may be advantageous are now further briefly described below.

Cell types for administration in cell therapies can be allogeneic or autologous. Further, the cell therapies may be modified or unmodified. The cell therapies may be any combination of allogenic or autologous that are modified or unmodified. Cell therapies may include hematopoietic stem cell transplant, whole blood transfusion, serum transfusion or fractions thereof, all of which included natural cell populations and proportions thereof. Cell therapy treatment strategies also include isolation and transfer of specific stem cell populations, administration of effector cells, induction of mature cells to become pluripotent cells, and reprogramming of mature cells. Administration of large numbers of effector cells may benefit cancer patients, transplant patients with unresolved infections, and patients with chemically destroyed stem cells in the eye.

Cell therapies associated with the present invention include the following approaches: (i) therapy with immune cells such as dendritic cells which are designed to activate the patient's own resident immune cells (e.g. T cells) to kill tumour cells, and (ii) direct infusion of immune cells such as T cells that find, recognize, and kill cancer cells directly. In both cases, therapeutic cells may be harvested and prepared in the laboratory prior to infusion into the patient. Immune cells, for example T cells, can be selected for desired properties and grown to high numbers in the laboratory prior to infusion. Challenges with these cellular therapies include the ability of investigators to generate sufficient function and number of cells for therapy.

The therapy may also be a therapy comprising the combination of both gene and cell therapies. Specifically, the therapy may comprise genes which encode for artificial receptors, which, when expressed by immune cells, allow these cells to specifically recognize cancer cells. This increases the ability of these gene modified immune cells to kill cancer cells in the patient. One example of this approach is the gene transfer of a class of novel artificial receptors called “chimeric antigen receptors” (CARs), into immune cells, typically a patient's own immune cells, e.g. T cells, which are then referred to as “CAR-T” cells. Accordingly the therapy may be a T cell therapy, wherein the T cell is genetically modified to express tumour specific CARs.

Of particular note as therapies of the present invention are cell-based therapies, including adoptive transfer of tumour infiltrating lymphocytes (TILs), or genetically engineered T cells, and T cell-engaging soluble bi-specific reagents, such as BiTEs and ImmTACs.

Chimeric antigen receptors (CARs) may mediate signalling through the zeta subunit of the TCR signalling complex. A major limitation of these CARs is the poor persistence of the CAR-engineered T cells in vivo; CAR persistence correlates with tumour regression in patients with advanced metastatic cancer. CARs may include additional co-stimulatory signalling domains, such as those from CD28, CD27 and 41-BB, all designed to enhance immune activation and T cell persistence.

CAR-engineered T cells may be targeted to the B cell antigen CD19.

The therapy may be TCR affinity-enhanced T cells, optionally specific for an epitope of NY-ESO-1.

The therapy may by a soluble bispecific molecule. The manufacture of soluble biologics can be considerably less expensive and time-consuming than cell-based therapies. Typically, T cell-engaging biologics are bi-specific fusion proteins that combine high-affinity TAA recognition—either antibody or TCR-based—with T cell activation, (usually via an anti-CD3 scFv antibody fragment), resulting in an activation that is independent of the T cells' natural specificity. Of all the antibody-based approaches, Triomabs are the most advanced, with catumaxomab recently on the market. These reagents incorporate an additional fragment crystallisable (Fc) component to enable the formation of a bridge between three cells: tumour target cell, T cell and accessory cell (macrophage, dendritic cell or natural killer (NK) cell). Triomabs targeting EpCam or Her2 are currently in Phase 1-3 clinical trials in various solid tumour indications. T cell-engaging antibodies (BiTEs) contain two single-chain variable fragments (scFv) produced as a single polypeptide chain. The most advanced BiTE—a CD19 targeting agent—resulted in an 80 percent response rate in a Phase 2 trial in patients with acute lymphoblastic leukaemia. Also in early phase clinical development are the closely related dual affinity retargeting antibodies (DARTS), which are produced as separate polypeptides joined by a stabilising interchain disulphide bond, and tetravalent tandem diabodies (TandAbs), in which the antibody fragments are produced as non-covalent homodimers folded in a head to-tail arrangement. Unlike antibodies, TCR-based biologics potentially access the entire repertoire of target antigens; however, progress has historically been limited because of the challenges of producing soluble TCRs. Immune-mobilising monoclonal TCRs against cancer (ImmTACs) provide a solution. ImmTACs comprise a soluble TCR, stabilised by a novel interchain disulphide bond and engineered to possess picomolar affinity for pMHC, fused to anti-CD3 scFv, to trigger potent T cell-mediated tumour cell killing in vitro and in vivo. ImmTACs represent the first generation of TCR-based soluble agents with picomolar affinities for their pMHC, overcoming one of the most pertinent obstacles to T cell tumour recognition. The furthest advanced ImmTAC is a gp100 peptide targeting agent which is currently being tested in a Phase 1/2 clinical trial in metastatic melanoma patients: some promising preliminary data is emerging from these studies.

Yet other approaches to harnessing the inherent power of the patient's immune system are focused on further improving the safety and clinical success of redirected T cell therapies. Approaches include better understanding in choosing a suitable target as well as an improved understanding of target expression in cancers and normal tissues. This is of paramount importance, particularly for TCR-based targeting systems that can access a vast array of potential antigens. In parallel, the application of comprehensive pre-clinical tests using more elaborate in vitro and in silico tools to predict clinical safety and toxicity is crucial to ensure the progression of the most promising pipeline candidates. Indeed, an effective preclinical pathway has recently been described for TCR-based therapeutics. Also, the implementation of an ‘abort’ mechanism for adoptively transferred T cells, such as an inducible suicide system, could improve clinical safety.

Given the complexity of cancer and its ability to evade the immune system, successful treatment (for example tumour eradication or inhibition of tumour growth or metastasis) will likely require a combination of therapies. Combination therapies may include two or more of the therapies mediated by NFAT activated T cells described herein, or combining one or more such therapies with another agent(s) for example chemotherapies or antibodies, (such as those chemotherapy agents discussed below) may improve efficacy and response durability. The combined therapy may provide an additive or more preferably, a more than additive effect compared to the use of the individual agents alone. For example potential synergies between immune checkpoint antibody inhibitors such as anti-PD1 and redirected T cells may prove persistently more active as a consequence of inhibiting T cell negative regulation. Any combination may include molecules that possess immunostimulatory properties.

The current invention is focused on selectively controlling the pharmacokinetics of a broad NFAT inhibitor such that the entire GIT, or in certain embodiments at least the colon, is exposed to the NFAT inhibitor to protect against GI ‘off target’ immune-driven adverse events and that the kinetics may be future modulated to provide the systemic exposure to protect against non-GI ‘off target’ effects without impacting on the ‘on target’ effects. This is achieved by the development of formulations that release a solubilised NFAT inhibitor, e.g. cyclosporine, throughout the GIT or at least in the colon.

In addition to the T effector cells—mainly Th1, Th2 and Th17—which, when activated, induce inflammation, a positive property of which is direct or indirect killing of cancer cells, another type of T cell, the T regulatory cell, provides a counterbalance. Thus, it would be expected to be beneficial if T effector cell activation can be controlled to provide ‘on target’ effects while a combination of an NFAT inhibitor to prevent ‘off target’ effects through the reduced activation of ‘off target’ T cells and preferential activation of T reg cells, to provide an improved balance of Treg:Teff cells while also an appropriate balance of ‘on target’ and ‘off target’ effects.

It has been reported that NFAT Is Required for Foxp3 Expression in iTreg or adaptive Treg which are generated from conventional naïve CD4+ cells in peripheral tissues. With respect to iTreg, NFAT2 was reported to bind CNS1 of Foxp3, an element that is considered to be crucial for iTreg generation in gut-associated lymphoid tissues. Using knock-out mice, the dependence of FoxP3 expression on NFAT2 in comparison with NFAT1 and NFAT4 was analysed. It was shown that combined with anti-CD3/28 and IL-2, TGF-6 induced robust Foxp3 expression in WT CD4+CD25-T cells, whereas induction was moderately diminished in the absence of NFAT2 and but more so when both NFAT1 and NFAT2 were missing. Further analysis of NFAT1 single- and NFAT1-NFAT4 double-deficient CD4+CD25-T cells showed that lack of one family member led to some reduction of Foxp3-expressing cells while loss of two members almost abrogated iTreg induction. The same group demonstrated that pharmacological inhibition of all NFAT members by cyclosporine A completely blocked Foxp3 induction in naïve CD4+ T cells during stimulation with anti-CD3/28 plus TGF-6 and IL-2. Using a thymocyte/T cell-specific Nfat2 knockout, the development of thymus-derived nTreg in the absence of NFAT2 was analysed. The data showed that the frequency of Foxp3+CD25+ nTreg among the CD4+ cell population in thymus, spleen, and lymph nodes (LN) were not affected by deficiency of any individual or combination of NFAT members. It was summarised that while nTreg develop irrespective of NFAT expression, the peripheral development of iTreg crucially rely on high NFAT levels with permissiveness for individual family members. While these data highlight the dependency of iTreg on NFAT transcription factors to develop and express FoxP3, once differentiated into nTreg or iTreg the suppressor function can be exerted even in the presence of low levels of NFAT. Supported by the finding that combined deletion of two of the three NFAT family members expressed in T cells barely impairs Treg suppressive activity which indicates that either minimal levels of NFAT activity suffice for regulatory function or that suppressive capacity is even independent of NFAT, this group concluded with the implication that high NFAT activity should be avoided for Treg function. On this basis, the authors proposed a bias against calcinuerin inhibitors. It was proposed that the calcineurin inhibitors CsA and FK506 were less preferred and new therapeutics such as R11-VIVIT and MCV1 that reduce NFAT activation specifically, thereby functionally inhibiting proinflammatory Teff but not Treg suppression should be advanced (Vaeth et al. PNAS, Oct. 2, 2012; vol. 109; no. 40; 16263).

The same group highlighted that pan-NFAT pharmacological inhibition using cyclosporine or tacrolimus was effective in reducing the ‘off-target’ adverse events associated with the immune-oncology therapeutic allogenic hematopoietic stem cell transplant approach. The ‘on target’ beneficial effects of this immune-therapy is due to activated T cells attacking tumour cells. The ‘off target’ adverse events are caused by activation and expansion of the allogeneic T lymphocytes attacking various tissues and organs. Based on previous reports that Tregs mitigate the ‘off target’ effects while maintaining ‘on target’ effects and drawing on the earlier NFAT knock-out investigations that highlighted the essential role of NFAT for Teff, but not Tregs, this group studied the specific contribution of individual NFAT family members to the immune pathogenesis of the ‘off target’ effect as well as the impact of NFATs on activated T cell antitumor activity. It was found that allogenic donor T cells deficient in NFAT not only reduced proliferation, target tissue homing, and impaired effector function, but conversely, increased Foxp3+ Treg frequencies following allogenic hematopoietic stem cell transplant. This work demonstrated that NFAT-deficient Tregs were fully suppressive and protected from ‘off target’ effects. Previous studies demonstrated that the spatial-temporal NFAT T cell activation pattern during ‘off target’ was strongest in the gastrointestinal tract, a primary target organ for ‘off target’ effects and that this was contemporaneous with inflammatory signals. It was proposed that this links with the identification through these NFAT knock-out studies that NFAT is functionally essential for target tissue homing via up-regulation of the gut-homing receptor α4β7-integrin. This report suggested that Tregs operating largely independent of NFAT and that cyclosporine treatment perturbs Treg function in an indirect manner, due to impaired IL-2 production of effector T cells. The authors concluded that other NFAT inhibitors with higher specificity for specific NFATs could modulate the ‘off target’ effects that would limit the severe side effects associated with pan-NFAT inhibitors such as cyclosporine, while at the same time not negating the ‘on target’ efficacy associated with T cell therapies. It was proposed that the use of cyclosporine, which can reduce both the ‘off target’ and ‘on target’ effects could be supplemented with low-dose IL2 to maintain the ‘on target’ effects (Vaeth et al.; PNAS; Jan. 27, 2015; vol. 112; no. 4; 1129). The above teaches away from a pan-/non-selective NFAT inhibitor and that maintainance of Treg function in the presence of cyclosporin will benefit from exogenous IL2. The current invention demonstrates that the selective distribution of a pan-NFAT inhibitor (in the absence of exogenous IL2) improves survival, modulates cytokine expression in target organs and improves the Treg;Teff balance in a model of NFAT-activated Tcell therapy.

Accordingly, in an embodiment of the invention IL2 (for example exogenous IL2) is not co-administered with the composition of the invention, co-administration optionally referring to simultaneous, sequential or separate administration of the IL2.

Therapies Mediated by NFAT Activated T Cells

The invention may find application whenever any form of T cell engaging therapy is employed in which T cells are activated when directly or indirectly bound to a target disease antigen via a receptor at the surface of the T cells. This receptor may be a naturally occurring T cell receptor, e.g. activated by a vaccine comprising a target antigen, e.g. a portion of an identified tumour antigen of interest, or a modified T cell receptor, e.g. directed against a tumour antigen or e.g. a high affinity T cell receptor. Other T cell engaging therapies which are envisaged may especially benefit from application of the invention include as noted above use of bispecific T cell engagers such as bispecific antibodies and CAR-T therapies.

The therapy mediated by NFAT activated T cells may be any of the therapies discussed herein and below. Optionally, the therapy is a bispecific T cell engager (also referred to as a bispecific T cell engaging therapy), a CAR cell therapy (e.g. a CAR-T cell therapy), or an immune checkpoint blockade therapy.

Bispecific T Cell Engagers

Bispecific T cell engagers, such as bispecific antibodies may bind both a T cell antigen and a disease antigen, e.g. a tumour antigen, or a T cell receptor and a disease antigen, e.g. a tumour antigen. Such bispecific T cell engagers have been of much interest, especially in relation to treating various cancers. Construction of such bispecific antibodies is well-known and includes use of linked antibody fragments, e.g. combined scFvs. By way of example, the therapeutic antibody blinatumomab is a CD19/CD3 bispecific T cell engaging antibody. In clinical trial its use has been reported to give rise to CRS. Reported attempts to date to manage such CRS occurrence have focussed on steroid use or additional use of tocilizumab (see for example, Bouhassira et al. (2015) Expert Opin. Biol. 15, 403-416). The anti-CD3 antibody is a strong activator of NFAT. Activation of NFAT via this pathway is inhibited by the inhibitor of NFAT activation. Accordingly, the composition of the present invention may be for use in a therapy comprising an anti-CD3 antibody or a fragment thereof.

As indicated above, alternatively it is known to link an anti-CD3 scFv with a monoclonal TCR to provide therapeutic bispecific T cell engagers. As in the case of bispecific antibodies, such bispecific constructs are of particular interest in relation to cancer treatment. For example, such a bispecific construct with a high-affinity engineered TCR to an HLA-A2 restricted peptide from the melanoma—associated antigen gp-100 is currently under investigation for the treatment of malignant melanoma. Similar constructs are envisaged with wide applicability in the field of cancer treatment. Again, the present invention may find use as an adjunct therapy or prophylactically in any clinical application where therapeutic benefit of the T cell targeting and activation also automatically comes with risk of unavoidable accompanying side effects, especially CRS and symptoms associated with gastrointestinal inflammation.

The therapy mediated by NFAT activated T cells may be a bi-specific antibody selected from: Blinatumomab [CD19 and CD3 III (ALL)], MEHD7945A [HER3 and EGFR II (colorectal cancer, head and neck cancer], ABT-122 [TNF and IL-17 II (rheumatoid arthritis)], ABT-981 [IL-1α and IL-1β II (osteoarthritis)], SAR156597 [IL-4 and IL-13 II (IPF)], MM-111 [ HER2 and HER3 II (gastric cancer)], IMCgp100 [a monoclonal T cell receptor anti-CD3 scFv fusion protein, GP100 and CD3 II (melanoma)], R05520985 [ANG2 and VEGFA II (colorectal cancer)], XmAb5871 [CD19 and CD32B I/II (rheumatoid arthritis)], COVA322 [TNF and IL-17A (psoriasis)], ALX-0761 [IL-17A and IL-17E I (psoriasis)], AFM13 [CD30 and CD16A I (Hodgkin's lymphoma)], AFM11 [CD19 and CD3 I (non-Hodgkin's lymphoma)], MEDI-565 [CEA and CD3 I (GI adenocarcinoma)], Ertumaxomab [HER2, CD3 and FcR I (solid tumours)], MGD006 [CD123 and CD3 I (AML)], MGD007 [GPA33 and CD3 I (colorectal cancer)], LY3164530 [MET and EGFR I (advanced cancer)].

The bispecific T-cell engagers may be multivalent, for example a trivalent or quadrivalent antibody or protein. For example, the therapy may be a tetravalent tandem diabody (TandAb), one example of which binds to the CD3 recptor of a T cell with two of its binding sites and uses the other two binding sites to bind to a receptor on the tumour (e.g. via a CD 19 or EGFRvIII receptor). Examples of a tetravalent tandem diabody include AFM11 and AFM21. The bispecific T-cell engager may be trivalent, for example a protein or antibody wherein, for example one binding site binds to a receptor on the T cell (e.g CD3) and the other two binding sites bind to two different receptors on the target tumour. Targeting two different sites on a tumour is expected to provide greater selectivity and/or efficacy of the T cell engaging therapy.

The therapy may be a multivalent antibody, including tetravalent antibodies, for example AMv-564 (Tetravalent antibody mimetic, a CD33/CD3 being developed by Amphivena Therapeutics, Inc.)

High Affinity T Cell Receptors (TCRs)

The therapy mediated by NFAT activated T cells may be instigated by T cells engineered with high affinity T cell receptors which can recognise tumour associated antigens. Examples of such TCRs include a TCR selected from NY-ESO-1 TCR 1, HPV-16 E6 TCR1, HPV-16 E6 TCR, MAGE A3/A6 TCR1, MAGE A3 TCR1, SSX2 TCR1, NY-ESO TCR (an engineered higher-affinity TCR targeting the NY-ESO-1 cancer testis antigen), MAGE-A-10 TCR (an engineered higher-affinity TCR targeting MAGE), BPX-701 (a TCR product candidate for solid tumours expressing the preferentially-expressed antigen in melanoma, or PRAME), ATTCK20 (Antibody-Targeted Tumour Cell Killing 20 (ATTCK20) is a combination of a patient's antibody-coupled T-cell receptor (ACTR) T-cells administered with rituximab, a monoclonal antibody targeting CD20. ACTR is a chimeric protein that combines components from receptors normally found on two different human immune cell types, natural killer (NK) cells and T-cells, in order to create new cancer cell killing activity. ATTCK occurs when T-cells expressing an ACTR engage a tumour-targeting antibody on the surface of a cancer cell).

CAR Therapies

The CAR therapy may be a CAR-immune cell therapy for example a CAR-T. Administration of a composition comprising an inhibitor of NFAT activation in accordance with the invention is also viewed of particular interest in relation to both autologous CAR-T therapies and allogenic CAR-T therapies, especially such therapies aimed at tackling haematological malignancies, e.g. B cell acute lymphoblastic leukaemia (B-ALL), chronic lymphocytic leukaemia (CLL) and acute myelogenous leukaemia (AML).

In such therapies, the receptor for the target antigen is an engineered chimeric receptor presented by the T cells comprising an extracellular antigen-binding portion commonly derived from an antibody, e.g. an ScFV, linked by a spacer to a transmembrane domain and T cell stimulatory domains, commonly a CD3-zeta domain as required for normal T cell activation and at least one co-stimulatory domain, e.g. a CD28 and/or 4-1BB signalling domain. For example, CAR-T cells expressing a fusion protein comprised of an anti-CD19 monoclonal antibody derived ScFv fused with CD28 costimulatory and CD3-zeta chain signalling domains are receiving much attention in relation to B-ALL patients. Such engineered chimeric receptors have the advantage of avoiding need to consider HLA restriction in target recognition and have been shown to effectively harness the normal beneficial mechanisms of T cell activation; it has been shown that antigen-binding will result in NFAT activation. However, as already noted above, CRS is a well-documented possible undesirable side effect of such therapy which can have life-threatening consequences.

In patients with leukaemias, infused CAR-T cells may be intensively activated after encountering a large amount of cancer cells in the peripheral blood. The CAR-T cells will proliferate which may potentiate CRS or add to the risk of CRS. Moreover, it is possible that pre-conditioning before CAR-T cell fusion may add considerably to the risk of CRS through cytokine production. As noted above, pre-conditioning by chemo- and/or radiotherapy may be associated with local inflammation in the GI tract with production of pro-inflammatory cytokines which result in differentiation of naïve T cells into activated T cells. It is also known that NFAT up regulates the gut-homing receptor α4β7-integrin on activated T cells.

Hence, inflammatory cytokine monitoring has become standard in carrying out CAR-T cell adoptive therapy. A dose escalation strategy may be employed to reduce the risk of problematic CRS arising, but it is difficult to judge an appropriate starting dose and therapeutic efficacy may be curtailed. Other means previously trialled for suppressing CRS are far from ideal. For example, corticosteroids such as methylprednisolone have been used in patients with mild and moderate CRS but affect CAR-T cell efficacy. As noted above, IL-6 receptor antibody directed therapy has more recently been suggested as favourable to suppress CRS in CAR-T therapy, but use of such a recombinant biologic has a high associated cost. The invention advantageously targets NFAT—expressing activated T cells in the GI tract which is separate from the normal main required site of action of CAR-T cells, e.g. systemically against a haematological malignancy, but can be anticipated to be a key site for development of the unwanted complication of CRS. Furthermore, the invention can employ an inhibitor of NFAT activation, preferably for example cyclosporin A, formulated for administration to the GI tract, which renders even routine prophylactic use of the inhibitor of NFAT activation plausible. In contrast, currently CRS associated with CAR-T therapy and other T cell therapies is normally only the subject of monitoring with a view to intervention with for example a steroid treatment when the CRS is considered severe.

The CAR-T therapy may target any disease associated antigen, for example a tumour associated antigen. For example, the CAR therapy may target an antigen selected from: Carbonic anhydrase IX (CAIX), CD19, CD20, CD22, CD30, CD33, CD38, CD44 (particularly variants 7/8), CD123, CD138, carcinoembryonic antigen (CEA), EGFR, EGFRvIII, erb-B2, erb-B3, erb-B4, WT1, c-Met, FAB, GD2, GD3, melanoma antigen family A1 (MAGE-A1), protein melan-A (melanoma antigen recognized by T cells 1 or MART-1), glycoprotein 100 (gp100), mesothelin, mucin 1, cell surface associated (MUC1), NY-ESO1, Prostate stem cell antigen (PSCA), prostate specific membrane antigen (PSMA), L1 cell adhesion molecule (L1CAM; CD171), MUC16(ecto), ROR1, VEGF-R2 and KDR (Tumour neovasculature), EGP-2, EGP-30, IL-13R-a2, k-light chain, TNFRSF17 (BCMA; CD269, SLAM family member 7 (SLAMF7 or CS1)) and Epstein Barr virus (EBV) antigens. Further examples of CARs include those described “In-cell immunotherapy: looking forward” Corrigan-Curay et al. Mol. Ther. 2014 September; 22(9):1564-74 and “chimeric antigen receptor T cell therapy to target hematologic malignancies” Kenderian et al. Cancer Res. 2014 November 15; 74(22):6383-9.

The CAR-T therapy may target an antigen selected from: CD19, CD20, and CD123.

Autologous CAR-T Therapies

The therapy may be an autologous CAR-T for example selected from CD19 CAR1, KTE-C19 CAR, EGFRvIII CAR, JCAR015 (CD19), JCAR017 (CD19), JCAR014 (CD19), BPX-401 (CD19) CBM-C19.1, CAR-T CD19, CTL109 (CD19) JCAR018 (CD22), JCAR023 (L1-CAM), JTCR016 (WT-1), a CAR-T directed to MUC16, for example IL-12 secreting, MUC-16(ecto) CAR T cells CAR-T directed to ROR1, BPX-601 (a CAR T product in development for the treatment of solid tumours overexpressing the prostate stem cell antigen, or PSCA, bb2121 (CAR-T cell therapy against tumour necrosis factor (TNF) receptor superfamily member 17 (TNFRSF17; BCMA; CD269)), CAR-T CD30 (CAR T cells specific to the CD30 antigen), CAR-T EGFR and CART-meso (a CAR-T cell directed against mesothelin)

Allogenic CAR-T Therapies

The therapy may be an allogenic CAR-T Therapy, for example selected from UCART19, UCART123, UCART38, UCARTCS1 and EBV-CTL

Immune Checkpoint Blockade Therapy

Immune checkpoint blockade therapy is another form of therapy mediated by autologous T cells but in this case unmodified T cells; one or more agents, e.g. antibodies, are employed to inhibit one or more known T cell inhibitory pathways.

T cell exhaustion in chronic viral infections has long been known to be linked to T cell exhaustion mediated by operation of such pathways. PD-1 and other inhibitory receptors such as LAG-3, 2B4 and Tim-3 act, at least in part, synergistically, contributing via non-redundant signalling pathways to establishment of T cell exhaustion. Thus, inhibitory-receptor mediated exhaustion is “tuned” by the availability of ligands in the environment. It is furthermore now known that T cells in the context of established progressing cancers exhibit an exhausted state similar to that observed in chronic infections due to high tumour-antigen load and immunosuppressive factors in the tumour micro-environment. T cells isolated from human tumours as well as experimental tumour models share many phenotypic and functional characteristics of exhausted T cells in chronic infections: tumour-infiltrating CD8 T cells are impaired in production of effector cytokines, express inhibitory receptors including PD-1, LAG-3, 2B4, TIM-3, CTLA-4, and display alterations in signalling pathways described for exhausted T cells.

Against this background, blockade of negative checkpoint receptors has emerged as a highly promising approach for treatment of cancers, especially antibody blockade of PD-1:PD-L1 interaction and anti-CTLA antibodies. Ipilimumab, a CTLA-4 blocking monoclonal antibody was the first FDA approved cancer immunotherapy for treatment of melanoma. However, reversing T cell hypo-responsiveness by PD-1 and/or or CTLA-4 blockade comes at cost: adverse immune toxicities, some serious and even fatal, have been observed in some patients (Schietinger and Greenberg (2014) Trends Immunol. 35, 51-60). Clinical trial of the anti-PD1 monoclonal antibody Lambrolizumab in a total of 135 patients with advanced melanoma reported promising results with common adverse events largely being designated low grade. However, the study was not without observation of adverse symptoms consistent with excessive production of cytokines and symptoms consistent with GI tract inflammation (Hamid et al. (2013) New Eng. J. Med. 369, 134-144). However, it needs to be borne in mind that such adverse symptoms are an innate risk of reversing T cell hypo-responsiveness and are difficult to eliminate entirely or predict as regards severity. The invention is envisaged as an advantageous means of managing this risk by administration of an inhibitor of NFAT activation, especially for example cyclosporin A to the GI tract. Importantly, such administration may enable prophylactic management of such risk a realistic and cost-effective option without jeopardising the therapeutic benefit of a relatively expensive biologic for tackling a cancer, especially in the context of therapeutic targeting of a haematological malignancy.

Examples of Checkpoint inhibitors that may be used in conjunction with the composition comprising the inhibitor of NFAT include, for example anti-PD-1/anti-PD-L1 inhibitors; antibodies targeting lymphocyte-activation gene 3 (LAG3; CD223); antibodies targeting glucocorticoid-induced tumour necrosis factor receptor (TNFR)-related protein (GITR; TNFRSF18)/Treg stimulators; anti-CTLA-4 receptor inhibitors; and anti-TIM-3 receptor inhibitors.

The therapy mediated by NFAT activated T cells may be for example, an anti-PD-1/anti-PD-L1 inhibitor selected from: REGN2810, Opdivo (nivolumab, a human IgG4 mAb against PD-1), Keytruda (pembrolizumab), humanized IgG4 mAb against PD-1, MED14736 is a human IgG1 mAb targeting PD-L1, anti-PD-L1 antibody MPDL3280A and PDR001 (PDR1).

The therapy mediated by NFAT activated T cells may be for example, an antibody targeting lymphocyte-activation gene 3 (LAG3; CD223), for example LAG525.

The therapy mediated by NFAT activated T cells may be for example an anti-CTLA-4 receptor inhibitor selected from: Yervoy (ipilimumab, a human mAb against CTLA-4 receptor) and tremlimumab, a human mAb against CTLA-4 (CD152).

The therapy mediated by NFAT activated T cells may be for example, an anti-TIM-3 receptor inhibitors for example MBG453.

It is also worthy of note that there is now much evidence linking Th-17-associated cytokine genes IL-17A and IL-17F and responsiveness of lymphoid cells to IL-23 with the etiology of inflammatory diseases including inflammatory bowel diseases such as ulcerative colitis and Crohn's' Disease (Geremia et al. (2011) J. Exp. Med. 208, 1127-1133; Liu et al. (2009) World J. Gastroenterol. 15, 5784-5788) In the case of colorectal cancers, chemotherapy has been reported to induce stromal cells to secrete high levels of IL-17A, a matter also of note in the development of oral small molecule antagonists of RORyt—the key transcription factor for driving the differentiation of IL-17A/F producing T helper lymphocytes (Th 17 cells). Such RORyt antagonists are now being developed by Visionary Pharmaceuticals for use in treating various Th17 cell inflammatory diseases. Moreover, cyclosporin A has previously been suggested to have clinical efficacy in treatment of steroid-resistant inflammatory conditions through attenuation of Th17 cells and IL17 production (Schweiz-Bowers et al. (March 2015) PNAS 4080-4085)) Hence, use of an inhibitor of NFAT activation in accordance with the invention, especially cyclosporin A formulated for oral delivery to the GI tract, might be usefully considered either alone or in conjunction with a RoRyt antagonist to reduce or prevent symptoms locally in the GI tract associated with GI inflammation for example, symptoms associated with inflammatory bowel disease, for example colitis.

It is also worthy of note that intestinal epithelial cell (IEC) apoptosis has been reported to contribute to ulcerative colitis and therapies that target the inflammatory cytokine TNF have been found to inhibit IEC apoptosis in patients with IBD Qiu et al. (2011) J. Clin. Invest. 121, 1722-1732). This may contribute to the effectiveness of use of inhibitors of NFAT activation as now proposed.

Furthermore, certain therapies mediated by NFAT-activated T cells contemplated by the invention cause apoptosis in the GIT. Cellular apoptosis in the GIT can manifest sypotmatically as diarrhea and inflammation. As with the paragraph above, the composition of the present invention inhibit apoptosis in the GIT further contributing to the effectivenesss of the therapy.

T cell therapies of interest in connection with the present invention are often employed as bridging therapies to allo-HCT treatment. It will be recognised that an important advantage of the present invention is that it may be employed to treat (ameliorate or prevent) undesirable effects arising in conjunction with therapeutic NFAT-activated T cells and then administration of the same composition may be continued to ameliorate or prevent GvHD with subsequent implementation of allo-HCT treatment. Accordingly, the invention contemplates a composition comprising an inhibitor of NFAT activation for use to treat an undesirable effect occurring in conjunction with a therapy (optionally a T cell therapy, preferably a CAR-T cell therapy) mediated by NFAT activated T cells and for use to treat GvHD in a subsequent allo-HCT therapy. The undesirable effects may be selected from cytokine release syndrome (CRS) and symptoms associated with gastrointestinal inflammation, e.g. associated with inflammatory bowel diseases such as ulcerative colitis and Crohn's' Disease. The composition is administered to the gastrointestinal tract whereby the one or more undesirable effects are reduced or prevented with maintenance of effectiveness of the therapy.

The conditioning chemotherapy and/or radiotherapy associated with an allo-SCT will eradicate residual CAR-T cells. However, considering that the lifespan of infused CAR-T cells in many patients is <3 months, then withholding allo-SCT until after B-cell recovery, an indirect measure of loss of CAR-T cell function, will ensure that patients get the full benefit of CAR T cell-mediated killing of malignant B cells. Of a small number of patients treated, 70% of eligible patients received an allo-SCT after CAR-T cell therapy, and there have been no relapses reported to date (follow up ranging from 2 to 24 months), which is supportive of the potential of adoptive CAR-T cell therapy as a bridge to allo-SCT, improving the clinical outcomes of this disease for patients with few-to-no treatment options.

The current invention may further be for use during allo-SCT procedure and beyond.

The present invention may advantageously be used as a precursor to treatment with a biologic, where such biologics activate T cells, at least part of the activation mechanism being through NFAT activation.

Also, the current invention may be: for use with repeated infusions of the therapy (optionally a T cell engaging therapy (e.g. CAR-T cell therapy)); for use with the therapy (e.g. CAR-T cell therapy), wherein the therapy is in combination with other procedures; for use as a prophylactic; for use in the therapy, wherein the therapy is administered to patients with a high disease burden; for use with the therapy, wherein the therapy is in combination with systemic stimulators, including IL-6.

High disease burd, also referred to as high tumour-burden, generally refers to patients with advanced cancers for example a Stage I, Stage II, Stage III or Stage IV cancer. High disease burden is well known to those of skill in the art and may for example be defined as a patient having >40%, >50%, >60% or >70% blasts in bone marrow, optionally wherein the blasts are B-cell lymphoblasts. High disease burden may also be suitably defined using the TNM system as has been accepted by the Union for International Cancer Control (UICC) and the American Joint Committee on Cancer (AJCC). The TNM system is based on the size and/or extent (reach) of the primary tumour (T), the amount of spread to nearby lymph nodes (N), and the presence of metastasis (M). By way of example, a high disease burden may be a patient with cancer defined as a T2, 3, or 4, and/or N1, 2 or 3; and/or M1 in the tumour, mode, metastasis (TNM) staging system. For example a high tumour burden may be defined as a cancer that is: T4 N3 M1, T4 N3 M0, T4 N2 M1, T4 N1 M1, T4 N2 M0, T4 N1 M0, T3 N3 M1, T3 N3 M0, T3 N2 M1, T3 N1 M1, T3 N2 M0, T3 N1 M0, T2 N3 M1, T2 N3 M0, T2 N2 M1, T2 N1 M1, T2 N2 M0 or T2 N1 M0.

The above highlights just some of the ways NFAT-activated T cells are being harnessed to fight disease. More are under consideration, e.g. rescuing AP-1 signalling in exhausted T cells (Martinez et al. (2015) ibid). It has also been found that VEGF-A produced in the tumour microenvironment enhances expression of PD-1 and other inhibitory checkpoints involved in CD8′ T cell exhaustion, which could be reverted by agents targeting VEGF-A/VEGFR (Voron et al. (January 2015) J. Exp. Med. 212, 139-148). However, it is further emphasised that any such therapy relying on activation of T cells relies on a balancing act between gaining effective therapeutic effect without at the same time engendering undesirable adverse events in the patient, most notably CRS and symptoms associated with gastrointestinal inflammation.

Undesirable Effects

The undesirable effects that occur in conjunction with therapies mediated by NFAT-activated T cells that are treated by the composition of the present invention are cytokine release syndrome or symptoms associated with gastrointestinal inflammation. Optionally, the symptoms may be associated with an inflammatory bowel disease such as any of those disclosed herein. The gastrointestinal (GI) inflammation may be caused by activated T cell activity, optionally the GI inflammation is caused by activated T cell activity in a patient who has been subjected to radiation or chemotherapy. Accordingly, the GI inflammation may be caused by activated T cell activity in a patient with or without radiation- or chemotherapy-induced damage.

As indicated above, CRS is a well-recognised undesirable immune adverse event associated with activity of NFAT-activated T cells. It can vary greatly in severity. Problematic CRS rendering intervention desirable may be equated with the following criteria for diagnosis: (i) fever, especially fever persisting for 3 or more consecutive days (ii) elevation in level of at least one of the main seven associated cytokines (IFN-γ, IL-6, Flt-3L, Fractalkine, IL-5, IL-10 and GM-CSF) (iii) at least one clinical sign of toxicity such as hypotension and hypoxia (PO₂ of less than 90%). Neurological changes may be observed. For example, a 75-fold elevation in level of two of the main seven CRS-associated cytokines or a 250-fold elevation of one such cytokine may be a key measure for CRS diagnosis. More recently, it was observed that patients with severe CRS consistently showed increased levels of C reactive protein (CRP) of 20 mg/dl or more in serum when compared to patients with no or non-problematic CRS. Hence, CRP at about 20 mg/dl or more has been proposed as a serum biomarker for severe CRS [Patel et al. (2014) Immunotherapy 6, 675-678]. Raised serum CRP may thus be a useful marker, alone or in conjunction with other diagnostic markers as noted above, for administering an inhibitor of NFAT-activation in accordance with the invention or monitoring the effectiveness of its administration to the GI tract, e.g. when administered prophylactically.

While CRS may often be the principle or only undesirable effect of concern, as hereinbefore indicated symptoms of gastrointestinal inflammation can also be anticipated to be an undesirable effect of therapies mediated by NFAT-activated T cells. Such symptoms may equate with or be associated with a condition selected from: irritable bowel disease, Crohn's disease, ulcerative colitis, celiac disease, gastroenteritis, duodenitis, jejunitis, ileitis, peptic ulcer, pouchitis, Curling's ulcer, appendicitis, colitis, pseudomembraneous colitis, diverticulosis, diverticulitis, collagenous colitis, systemic inflammation optionally emanating from the GIT, colorectal carcinoma and adenocarcinoma. The invention may find particular application in reducing or preventing symptoms of ulcerative colitis or Crohn's disease.

The invention is applicable to reducing or preventing such undesirable effects in relation to any of a wide range of therapies mediated by T cells in which functional NFAT expression is present as will be further expanded upon below. Such a therapy may be a combined therapy where T cell activation is promoted by more than one means, e.g. combining T cells presenting a chimeric antigen receptor to a target antigen with a means for immune checkpoint blockade, e.g. an anti-PD1 antibody.

The invention may find application whenever any form of T cell engaging therapy is employed in which T cells are activated when directly or indirectly bound to a target disease antigen via a receptor at the surface of the T cells. This receptor may be a naturally occurring T cell receptor, e.g. activated by a vaccine comprising a target antigen, e.g. a portion of an identified tumour antigen of interest, or a modified T cell receptor, e.g. directed against a tumour antigen. The receptor may be a bispecific antibody which binds both a T cell antigen and a disease antigen, e.g. a tumour antigen. Construction of such bispecific antibodies is well-known and includes use of linked antibody fragments, e.g. combined scFvs. Administration of a composition comprising an inhibitor of NFAT activation in accordance with the invention is however viewed of especial interest in relation to CAR-T therapies, both autologous CAR-T therapies and allogenic CAR-T therapies, especially such therapies aimed at tackling haematological malignancies, e.g. B cell acute lymphoblastic leukaemia (B-ALL), chronic lymphocytic leukaemia (CLL) and acute myelogenous leukaemia (AML).

In such therapies, the receptor for the target antigen is an engineered chimeric receptor presented by the T cells comprising an extracellular antigen-binding portion commonly derived from an antibody, e.g. an ScFV, linked by a spacer to a transmembrane domain and T cell stimulatory domains, commonly a CD3-zeta domain as required for normal T cell activation and at least one co-stimulatory domain, e.g. a CD28 and/or 4-1BB signalling domain. For example, CAR-T cells expressing a fusion protein comprised of an anti-CD19 monoclonal antibody derived ScFv fused with CD28 costimulatory and CD3-zeta chain signalling domains are receiving much attention in relation to B-All patients. Such engineered chimeric receptors have the advantage of avoiding need to consider HLA restriction in target recognition and have been shown to effectively harness the normal beneficial mechanisms of T cell activation; it has been shown that antigen binding will result in NFAT activation. However, as already noted above, CRS is a well-documented possible undesirable side effect of such therapy which can have life-threatening consequence.

In patients with leukaemias, infused CAR-T cells may be intensively activated after encountering a large amount of cancer cells in the peripheral blood. The CAR-T cells will proliferate which may potentiate CRS or add to the risk of CRS. Moreover, it is possible that pre-conditioning before CAR-T cell fusion may add considerably to the risk of CRS through cytokine production. Hence, inflammatory cytokine monitoring has become standard in carrying out CAR-T cell adoptive therapy. A dose escalation strategy may be employed to reduce the risk of problematic CRS arising, but it is difficult to judge an appropriate starting dose and therapeutic efficacy may be curtailed. Other means previously trialled for suppressing CRS are far from ideal. For example, corticosteroids such as methylprednisolone have been used in patients with mild and moderate CRS but affect CAR-T cell efficacy. As noted above, II-6 receptor antibody directed therapy has more recently been suggested as favourable to suppress CRS in CAR-T therapy but use of such a recombinant biologic has a high associated cost. The invention advantageously targets NFAT-expressing activated T cells in the GI tract which is separate from the normal main required site of action of CAR-T cells, e.g. systemically against a hematologic malignancy, but can be anticipated to be a key site for development of the unwanted complication of CRS.

As indicated above, this may be particularly so where pre-conditioning is carried out to deplete autologous T cells. Pre-conditioning by chemo- and/or radiotherapy may be associated with local inflammation in the GI tract with production of pro-inflammatory cytokines and chemokines by innate and adaptive immune cellswhich result in differentiation of naïve T cells into activated T cells. It is also known that NFAT up regulates the gut-homing receptor α4β7-integrin. Furthermore, the invention can employ a chemical entity, preferably for example cyclosporin formulated for administration to the GI tract, for prophylactic use. In contrast, CRS is relation to CAR-T therapy and other T cell therapies is at present normally only the subject of monitoring with a view to intervention when considered severe.

Accordingly, the present invention can be for use wherein the therapy comprises a pre-conditioning regime to deplete autologous T cells in the patient. The pre-conditioning may be by chemo- and/or radiotherapy. The present invention may be for treating undesirable effects associated with gastrointestinal inflammation and CRS where the therapy mediated by NFAT activated T cells comprises a chemo- and/or radiotherapy pre-conditioning regime.

Cytokine release syndrome (CRS) can be characterised as being between mild (with fevers, chills, fatigue and headaches) all the way to life-threatening cases (with hypotension, tachycardia, pulmonary edema, altered mental status and seizures). The most severe cases require pressor support and mechanical ventilation. Generally these are associated with a concomitant rise in cytokines and CAR T cells. CRS symptoms may begin to manifest as early as 2 days after administration of the therapy (for example CAR T cell infusion).

The cytokine release syndrome (CRS) is a set of clinical toxicities including fevers, hypotension and neurologic changes associated with en masse T cell activation associated with the administered therapy, for example en-mass activation of CAR-T cells by their target antigen. The severe illnesses associated with this syndrome represent the main clinical limitation to adapting therapies such as CAR T cells to a larger population of patients. Toxicities observed during clinical trials of cell therapies such as CAR T cells include hypotension, fevers, fatigue, renal failure and obtundation. Cytokine elevations were seen coincident with the toxicities, so they are believed to be secondary to a cytokine release syndrome (CRS).

Patients presenting with CRS had many varied elevations in the 39 cytokines examined. CRS may be characterised by an elevation of cytokines, such as IL-2, IFN-g and IL-10, whereas others stand out as less expected; for example, IL-1b is produced by macrophages, dendritic cells, endothelial cells and hepatocytes, and IL-12 is produced by macrophages and dendritic cells.

The undesirable effect may be clinically significant, severe CRS (sCRS) associated with CAR T cell activation. sCRS is defined as consisting of: i) fevers for at least 3 consecutive days; ii) two cytokine maximum fold changes of at least 75-fold from baseline or one cytokine maximum fold change of at least 250-fold from baseline (out of a preselected group of seven cytokines found to be commonly elevated in patients with CRS); and iii) at least one clinical sign of toxicity such as hypotension (requiring at least one intravenous vasoactive pressor), hypoxia (PO2<90%), or neurologic disorders (including mental status changes, obtundation and seizures), Davila et al.

The severity of CRS and cytokine elevation correlates significantly with tumour burden at the time of CAR T cell infusion. Therefore, the invention contemplates administering a higher dosage of NFAT inhibitor to subjects known to have a high tumour burden. Alternatively or additionally, patients who receive an NFAT inhibitor (for example by administration of a composition of the invention) may receive a higher dose of the cell therapy, (e.g. CAR-T cells). This higher dose of cell therapy may be in conjunction with a higher tumour burden or may not (i.e. the higher dosage of cell therapy may be administered regardless of the tumour-burden prior to treatment). Patients without morphologic residual disease showed minor or undetectable cytokine elevations, whereas all the patients who developed sCRS had morphological residual leukaemia. Distinguishing the severity of CRS and determining when to treat the toxicities is essential to avoid premature, unnecessary intervention that could limit the efficacy of CAR T cells.

The dose of NFAT inhibitor may also be changed, as appreciated by one skilled in the art. The amount of NFAT inhibitor administered may be different as discussed elsewhere herein. The timing of the NFAT inhibitor may also be differed. For example, the NFAT inhibitor may be administered constantly (e.g. on each consecutive day optionally at the same time(s)) or the NFAT inhibitor may be administered in a pulsatile manner (e.g. on non-consecutive days).

A correlation between CRP and severity of CRS has been established, with the sickest patients reaching CRP elevations of 40-50 μg/ml. There was a significant difference in average CRP in patients with or without sCRS. Therefore, it is recommended that when CRP levels reach 20 μg/ml, patients should begin an intensive monitoring program because they are at risk of impending clinical toxicities. Accordingly, a composition of the invention may be administered to a patient when their CRP level is >5 μg/ml, >10 μg/ml or >20 μg/ml, for example from 5 to 40 μg/ml, 10 to 40 μg/ml, 5 to 30 μg/ml, 5 to 20 μg/ml, 10 to 30 μg/ml, 10 to 20 μg/ml.

The current invention will support the development of treatment algorithms that have the potential to enhance immuno therapeutic efficacy (for example CAR-T function) and outcome, while limiting the risk of CRS or similar responses, while also having the potential to enable a standardisation of a protocol for prophylaxis as well as intervention. Such treatment algorithms may include for example combination therapies and/or dosage regimens.

The composition of the invention is for use in reducing or preventing one or more undesirable effects. The undesirable effect may be selected from cytokine release syndrome and symptoms associated with inflammatory bowel diseases. The composition may comprise at least one further active ingredient, for example at least one immunosuppressant. In particular the undesirable effects may be selected from CRS and symptoms associated with an inflammatory bowel disease, irritable bowel syndrome, Crohn's disease, ulcerative colitis, celiac disease, graft-versus-host disease, gastrointestinal graft-versus-host disease, gastroenteritis, duodenitis, jejunitis, ileitis, peptic ulcer, Curling's ulcer, appendicitis, colitis, pseudomembraneous colitis, diverticulosis, diverticulitis, pouchitis, collagenous colitis, macorscopic colitis, diarrheal colitis, endometriosis, colorectal carcinoma and adenocarcinoma. The symptoms may also be associated with proctitis. The symptoms may be associated with primary sclerosing cholangitis, familial adenomatous polyposis, or perianal Crohn's, including perianal fistulae.

Dendritic cells may play a role in the undesirable effect. The composition of the invention may inhibit NFAT activation in dendritic cells. For example the therapy may comprise a modified dendritic cell such as DC-Ad GM-CAIX (Kite Pharma), dendritic cells which are modified to express a fusion protein consisting on GM-CSF and CAIX.

More generally immune cells, for example innate and adaptive immune cells, may play a role in the undesirable effect. The composition of the invention may inhibit NFAT activation in immune cells, for example immune cells in the GIT such as dendritic cells. The immune cells may be part of the therapy, for example allogenic cells, the therapy may comprise a modified dendritic cell such as those discussed in the preceding paragraph, or the immune cells may be autologous.

The cyclosporin compositions according to this aspect may be administered orally, for example to provide an instant release composition. Also contemplated is the administration of the composition to the GI tract rectally, for example in the form of an enema or suppository. Other routes of administration of the composition are also contemplated, for example the composition may be administered directly to the GIT, by for example intra-duodenal administration, intra-jejunal or intra-ileal administration. Such routes of administration enable the composition to bypass the stomach (and optionally other parts of the GI tract) for delivery to specific points in the lower GI tract. These routes of administration may be achieved using for example suitable tubing with an exit at the desired location within the GI tract. Suitably the tubing is inserted orally or nasally into the GI Tract. Alternatively, administration may be achieved by gastric tubing, or continuous or discontinuous percutaneous endoscopic gastrostomy (PEG) tubing. PEG is an endoscopic medical procedure in which a tube (PEG tube) is passed into a patient's stomach through the abdominal wall. This method of administration may be particularly suitable for patients that cannot take the drug orally due to for example dysphagia or sedation.

The composition may be a solid composition. The composition may be coated with an enteric coating (for example a delayed release polymer coating, an immediate release polymer coating) or no coating, the coating (or absence thereof) enabling the composition to be released at sites of the GIT, for example in the small intestine and/or the colon. The composition may comprise an oil phase, the oil phase optionally comprising the NFAT inhibitor.

The composition may also comprise a hydrogel forming polymer matrix. The composition may optionally comprise a surfactant. The composition may optionally comprise an oil phase. The oil phase is preferably dispersed in the hydrogel forming polymer matrix. The composition of the invention may be a composition comprising an NFAT inhibitor (optionally cyclosporin) and a hydrogel forming polymer matrix, and/or (preferably and) a surfactant and/or (preferably and) an oil phase, optionally being dispersed in the hydrogel forming polymer matrix. The surfactant may be any surfactant defined elsewhere herein. The surfactant optionally may be or may comprise a medium chain or long chain fatty acid mono- or di-glyceride or a combination thereof and may not comprise or may not be a polyethyleneglycol ether or ester. The composition may be a solid composition. The composition may be in the form of a dried bead. The composition may be in the form of a dried colloid. Preferably the composition is for oral administration.

Optionally, the NFAT inhibitor (optionally cyclosporin), the hydrogel forming polymer matrix, the surfactant and the oil phase are comprised within a core. Thus, the composition may comprise a core. Accordingly, the composition may comprise a core, wherein the core comprises cyclosporin, a hydrogel forming polymer matrix, a surfactant and an oil phase being dispersed in the hydrogel forming polymer matrix.

The core may be a dried colloid optionally formed by solidification of a liquid colloid, i.e. it may be a dried colloidal composition. The composition may be a solid colloid or the composition may be in the form of a solid colloid, i.e. it may be a solid colloidal composition. The liquid colloid may comprise a continuous phase which is or comprises a hydrogel-forming polymer and a disperse phase which is or comprises the NFAT inhibitor and an oil phase, wherein the liquid colloid further comprises a surfactant (also referred to as a first surfactant).

The solid colloidal composition of the invention may comprise a continuous phase which is or comprises a hydrogel-forming polymer matrix and a disperse phase which is or comprises cyclosporin A and an oil phase, wherein the colloidal liquid composition or the solid colloidal composition further comprise a surfactant (also referred to as a first surfactant) comprising or being a medium chain or long chain fatty acid mono- or di-glyceride or a combination thereof and not comprising or not being a polyethyleneglycol ether or ester.

In an embodiment the oil phase comprises a solution of the NFAT inhibitor. As such, the NFAT inhibitor may be dissolved in the oil phase, for example completely dissolved, substantially completely dissolved, or partially dissolved. Thus, the oil phase may comprise a solution of the NFAT inhibitor and some undissolved the NFAT inhibitor.

The NFAT inhibitor may be a calcinuerin inhibitor. The NFAT inhibitor may be lipid soluble. The NFAT inhibitor may be selected from: cyclosporin, cyclosporin derivatives, tacrolimus derivatives, pyrazoles, pyrazole derivatives, phosphatase inhibitors, S1P receptor modulators, toxins, paracetamol metabolites, fungal phenolic compounds, coronary vasodilators, phenolic adeide, flavanols, thiazole derivatives, pyrazolopyrimidine derivatives, benzothiophene derivatives, rocaglamide derivatives, diaryl triazoles, barbiturates, antipsychotics (penothiazines), serotonin antagonists, salicylic acid derivatives, phenolic compounds derived from propolis or pomegranate, imidazole derivatives, pyridinium derivatives, furanocumarins, alkaloids, triterpenoids, terpenoids, oligonucleotides, or peptides.

The NFAT inhibitor may be selected from: cyclosporin, tacrolimus, A 285222, endothall, 4-(fluoromethyl)phenylphosphate FMPP, norcantharidin, tyrphostins, okadaic acid, RCP1063, cya/cypa (cyclophilin A), isa247 (voclosporin)/cypa, [dat-sar]³-cya, fk506/fkbp12, ascomyxin/fkbp12, pinecrolimus/FKBP12, 1,5-dibenzoyloxymethyl-norcantharidin, am404, btp1, btp2, dibefurin, dipyridamole, gossypol, kaempferol, lie 120, NCI3, PD 144795, Roc-1, Roc-2, Roc-3, ST 1959 (DLI111-it), thiopental, pentobarbital, thiamylal, secobarbital, trifluoperazine, tropisetron, UR-1505, WIN 53071, caffeic acid phenylethyl ester, KRM-III, YM-53792, punicalagin, imperatorin, quinolone alkaloids compounds 1 and 3, impressic acid, oleanane triterpenoid compound 3, gomisin N, CaN₄₅₇₋₄₈₂-AID, CaN₄₂₄₋₅₂₁-AID, mFATc2₁₀₆₋₁₂₁-SPREIT, VIVIT peptide, R11-Vivit, ZIZIT cis-pro, INCA1, INCA6, INCA2, AKAP79₃₃₀₋₃₅₇, RCAN1, RCAN1-4₁₄₁₋₁₉₇-exon7, RCAN1-4₁₄₃₋₁₆₃-CIC peptide, RCAN1-4₉₅₋₁₁₈-SP repeat peptide, LxVPc 1 peptide, MCV1, VacA, A238L, and A238₂₀₀₋₂₁₃.

Preferably, the NFAT inhibitor is cyclosporin. Throughout this specification the term cyclosporin may be referring to the class of compounds or to cyclosporin A. Preferably, the use of cyclosporin is in reference to cyclosporin A.

The NFAT inhibitor (optionally cyclosporin) is suitably present in the composition in an amount of from about 5% to about 20%, from about 8% to about 15%, or from about 9% to about 14% by weight based upon the dry weight of the core or of the composition.

The NFAT inhibitor (optionally cyclosporin) is suitably present in the liquid composition in an amount of up to 10%, optionally from about 1% to about 10%, from about 2% to about 8%, from about 3% to about 6%, from about 3% to about 5% by weight of the liquid composition. Optionally the cyclosporin may be present in the liquid composition in about 4% by weight of the liquid composition.

The compositions described herein may be used to deliver the NFAT inhibitor locally to specific locations in the GIT, for example the solid compositions described herein, may be adapted to provide release of the NFAT inhibitor in at least the colon. The compositions may be used to provide the NFAT inhibitor locally in the GIT in an active form, for example in a solubilised form, thereby providing high concentrations of the NFAT inhibitor in an active (available) form within the GIT where it acts to reduce or prevent the undesirable effects of the therapy with maintenance of the therapeutic effect on the condition being treated such as cancer. The release of the NFAT inhibitor in an active form, for example a solubilised form, enables high concentrations of the NFAT inhibitor to be absorbed directly into the local tissues of the GIT, such as the colon. However, as described above, systemic exposure the certain NFAT inhibitors, particularly cyclosporin A, have a number of undesirable side effects. Therefore, a cyclosporin A composition which minimises systemic exposure to cyclosporin whilst maintaining therapeutically beneficial concentrations in the tissues of the GIT would be desirable.

The compositions of the invention may also be used to deliver the NFAT inhibitor to sites of the GIT that allow for systemic absorption and modulation of NFAT. The composition may be used to provide the NFAT inhibitor in a solubilised form, thereby providing high concentrations of NFAT inhibitor in an active (available) form. For example, the composition may be adapted to release the NFAT inhibitor in the stomach, small intestine (duodenum, jejunum or ileum), large intestine (cecum, colon, or rectum) or a combination thereof. Preferably, systemic absorption of the NFAT inhibitor can be achieved with a composition adapted to release the NFAT inhibitor in the stomach and/or small intestine (duodenum, jejunum and/or the ileum), preferably release is in the duodenum, jejunum and/or ileum.

The composition may comprise a coating to control or modulate release of the cyclosporin from the composition. Advantageously the coating is a polymeric coating to provide immediate, delayed and/or sustained release of the cyclosporin from the composition. Suitably such coatings are described in more detail below and include a coating which is or comprises a coating selected from a controlled release polymer, a sustained release polymer, an enteric polymer, a pH independent polymer, a pH dependent polymer and a polymer specifically susceptible to degradation by bacterial enzymes in the gastrointestinal tract, or a combination of two or more such polymers. In a particular embodiment the coating is or comprises a pH-independent polymer, for example a coating which is or comprises ethyl cellulose. In a further specific embodiment the coating is or comprises a pH-independent polymer, for example ethyl cellulose, and optionally a water-soluble polysaccharide, for example pectin or chitosan, or a combination thereof, particularly pectin.

In an embodiment the coating that is referred to in the preceding paragraph is an outer coating, also referred to as a second coating. The composition may optionally comprise a further coating, referred to as a sub-coat or a first coating. The respective polymers of the first coating and the second coating are different. Often the second coating does not have any polymer found in the first coating; for example, if the first coating comprises (e.g. is) a hydroxypropylmethyl cellulose, then the second coating will not also comprise a hydroxypropylmethyl cellulose. In addition the situation is contemplated where the first coating is or comprises a water-soluble ether or ester of a cellulose ether, the major component(s) (e.g. more than 50%) of the second coating is or comprises a different polymer to that of the first coating. Accordingly, the first and second coatings suitably provide two layers of material as part of the composition. It is to be understood that when the second coating comprises a mixture of components, minor components of the outer second coating may be the same as the material of the sub-coating. By way of example, when the first coating is or comprises HPMC and the second coating comprises ethyl cellulose, the ethyl cellulose may optionally further comprise a minor amount (e.g. less than 50%, 40%, 30% or 20%) of the first coating material, HPMC in this example. In such embodiments the first coating and the second coating are considered to be different.

In embodiments where the pharmaceutical composition does not comprise a second coating, the undesirable symptoms may be associated with conditions that affect the small intestine. Such compositions may be able to reduce or prevent symptoms associated with conditions selected from celiac disease or Crohn's disease.

The composition of the invention may comprise an NFAT inhibitor (optionally cyclosporin), a hydrogel forming polymer matrix, a surfactant and an oil phase being dispersed in the hydrogel forming polymer matrix. Optionally, the composition may further comprise a first coating, wherein the first coating is or comprises a water-soluble cellulose ether as described above and elsewhere herein. In addition to the first coating or alternatively to the first coating the composition may comprise a second coating. Optionally, the second coating is or comprises a coating, suitably a polymeric coating, to control or modulate release of the active ingredient from the composition. The polymeric coating may be as further described elsewhere in this specification.

Where the composition comprises a first coating and a second coating the second coating may be outside the first coating.

The composition may comprise: a core, wherein the core comprises an NFAT inhibitor (optionally cyclosporin), a hydrogel forming polymer matrix, a surfactant and an oil phase being dispersed in the hydrogel forming polymer matrix; a first coating outside the core, wherein the first coating is a water-soluble cellulose ether as described above and elsewhere herein; and a second coating outside the first coating, wherein the surfactant is as described herein. Throughout this specification “core” may refer to a core comprising cyclosporin, a hydrogel forming polymer matrix, a surfactant, as described herein, and an oil phase being dispersed in the hydrogel forming polymer matrix.

According to an embodiment of the invention, the surfactant optionally is a medium chain or long chain fatty acid mono-glyceride, di-glyceride or a combination thereof, the first coating is or comprises a water-soluble cellulose ether, and the composition further comprises a second coating outside the first coating wherein the second coating is or comprises a coating, suitably a polymeric coating, to control or modulate release of the active ingredient from the composition. The polymeric coating may be as further described elsewhere in this specification.

The first coating suitably may be or comprise a water-soluble cellulose ether. The water-soluble cellulose ether may be any cellulose ether or derivative of a cellulose ether, for example an ester of a cellulose ether that is soluble in water. Therefore, the water-soluble cellulose ether may be selected from: an alkyl cellulose; a hydroxyalkyl cellulose; a hydroxyalkyl alkyl cellulose; and a carboxyalkyl cellulose. Suitably the first coating is or comprises one or more water-soluble cellulose ethers selected from: methyl cellulose, hydroxyethyl cellulose, hydroxypropyl cellulose and hydroxypropylmethyl cellulose, and combinations thereof. In particular embodiments the first coating is or comprises a water-soluble hydroxypropyl methylcellulose. The water-soluble cellulose ethers and water-soluble derivatives thereof (e.g. water-soluble esters of a cellulose ether) present in the first coating (sub-coat) suitably form at least 20%, 40%, 50%, 60%, 70%, 80%, 85% or 90% by weight of the dry weight of the first coating.

In accordance with the present invention there is provided a pharmaceutical composition comprising a core and a first coating, wherein the core comprises cyclosporin, a hydrogel forming polymer matrix, a surfactant and an oil phase being dispersed in the hydrogel forming polymer matrix and the first coating comprises or is a water soluble cellulose ether and the first coating is present in an amount corresponding to a weight gain due to the first coating of from 0.5% to 20% by weight of the core, wherein the surfactant is as described herein, for example a medium chain or long chain fatty acid mono- or di-glyceride or a combination thereof and not comprising or not being a polyethyleneglycol ether or ester.

The first coating of the present invention modifies the release of the active ingredient from the composition. There would be an expectation that a coating on a composition would slow the rate of release of the active ingredient within a composition. One might reasonably expect this as coating the composition with additional material would provide an additional barrier to a dissolution medium coming into contact with the active ingredient in the composition. In contrast to this expected outcome, the compositions of the present invention comprise a coating comprising or being a water soluble cellulose ether that increases the rate of release of the active ingredient compared to a composition without the coating. In addition the coating of the present invention has the beneficial effect of maintaining the active ingredient in solution, whereas a comparable composition lacking the coating of the invention provides less of the active ingredient in solution as time progresses. Without wishing to be bound by theory, it is believed that the coating prevents precipitation of the active ingredient from solution, thereby maintaining a higher amount of the active in solution.

Throughout the present application active ingredient, active, and pharmaceutically active ingredient are used interchangeably and all refer to a NFAT inhibitor, optionally cyclosporin, preferably cyclosporin A.

The composition of the present invention may take any form known to the person skilled in the art. Preferably, the composition is an oral composition. The composition may be in the form of a single minibead or a multiplicity of minibeads. Accordingly the invention provides a multiplicity of minibeads of the invention. Similarly, the invention provides a multiple minibead formulation comprising a unit dosage form comprising a multiplicity of minibeads.

The invention also provides for a pharmaceutical composition comprising a core and a first coating, wherein the core comprises a NFAT inhibitor, a hydrogel forming polymer matrix, a surfactant and an oil phase being dispersed in the hydrogel forming polymer matrix and the first coating comprises or is a water-soluble cellulose ether and the first coating has a thickness of from 1 μm to 1 mm wherein the surfactant is as described herein, for example a medium chain or long chain fatty acid mono- or di-glyceride or a combination thereof and not comprising or not being a polyethyleneglycol ether or ester.

Any of the pharmaceutical compositions of the invention may comprise a further coating, referred to herein as a second coating. The second coating may be outside the first coating. The second coating may be or comprise a delayed release polymer. In any embodiment and any aspect of the invention the first and second coating may be different.

The invention therefore, contemplates a pharmaceutical composition comprising a core, a first coating and a second coating outside of the first coating, wherein the core comprises a NFAT inhibitor, a hydrogel forming polymer matrix, a surfactant and an oil phase being dispersed in the hydrogel forming polymer matrix, the first coating comprises or is a water soluble cellulose ether (for example HPMC), and the second coating comprises or is a delayed release polymer (for example ethylcellulose).

The composition of any aspect or embodiment of the invention may be in the form of a solid colloid. Furthermore, the core of a composition may be in the form of a solid colloid. The colloid comprises a continuous phase and a disperse phase. Suitable continuous phases and disperse phases which may be used to form the core are defined in more detail below and in the detailed description of the invention. The continuous phase may comprise or be the hydrogel forming polymer matrix. Hence, where the continuous phase is the hydrogel forming polymer matrix, the composition of the invention may take the form of a solid unit of the hydrogel forming polymer comprising a disperse phase. The disperse phase may be droplets dispersed in the continuous phase, or the hydrogel forming polymer matrix. The disperse phase may comprise or be the oil phase.

Thus, the invention provides a composition in the form of a solid colloid comprising a continuous phase and a dispersed phase, wherein the continuous phase comprises or is a hydrogel forming polymer matrix and the continuous phase is or comprises oil phase, wherein the composition further comprises an NFAT inhibitor (for example cyclosporin) and a surfactant. Preferably, the surfactant is a medium chain or long chain fatty acid mono- or di-glyceride or a combination thereof and does not comprise or is not a polyethyleneglycol ether or ester. The oil phase may comprise the cyclosporin in solution.

The composition may comprise a core in the form of a solid colloid comprising a continuous phase and a dispersed phase, wherein the continuous phase comprises or is a hydrogel forming polymer matrix and the continuous phase is or comprises oil phase, wherein the core further comprises a NFAT inhibitor and a surfactant. The oil phase may comprise the NFAT inhibitor, optionally in solution.

The continuous phase of a solid colloid composition or core is or comprises a hydrogel-forming polymer matrix. In embodiments the hydrogel-forming polymer matrix is or comprises a hydrocolloid, a non-hydrocolloid gum or chitosan. In a particular embodiment the hydrogel-forming polymer matrix is or comprises gelatin, agar, a polyethylene glycol, starch, casein, chitosan, soya bean protein, safflower protein, alginates, gellan gum, carrageenan, xanthan gum, phthalated gelatin, succinated gelatin, cellulosephthalate-acetate, oleoresin, polyvinylacetate, polymerisates of acrylic or methacrylic esters and polyvinylacetate-phthalate and any derivative of any of the foregoing; or a mixture of two or more such polymers. In a further embodiment the hydrogel-forming polymer matrix is or comprises a hydrocolloid selected from carrageenan, gelatin, agar and pectin, or a combination thereof optionally selected from gelatin and agar or a combination thereof. Particularly, the polymer of the hydrogel-forming polymer matrix is or comprises gelatin. In an embodiment, the hydrogel-forming polymer does not comprise a cellulose or a cellulose derivative, e.g. does not comprise a cellulose ether.

In this aspect of the invention the composition may be in the form of a solid colloid the colloid comprising a continuous phase and a disperse phase and the NFAT inhibitor may be in solution or suspended in the disperse phase. For example, the cyclosporin may be in solution in the disperse phase.

It is to be understood that the individual embodiments described above may be combined with one or more of the other embodiments described to provide further embodiments of the invention.

The first coating may be in contact with the core. The second coating may be on the first coating. In embodiments the first coating is in contact with the core and the second coating is on the first coating.

The second coating may be or may comprise a delayed release polymer and the delayed release polymer may be selected from an enteric polymer, a pH independent polymer, a pH dependent polymer and a polymer specifically susceptible to degradation by bacterial enzymes in the gastrointestinal tract, or a combination of two or more such polymers. Hence, the second coating may be any of the aforementioned delayed release polymers or any may be or possess the characteristics mentioned in relation to the delayed release polymer mentioned below.

In embodiments the delayed release polymer may be water-soluble or water-permeable in an aqueous medium with a pH greater than 6.5. The delayed release polymer may be or comprise a pH-independent polymer, for example ethyl cellulose.

In any aspect and any embodiment of the invention the water-soluble cellulose ether may be selected from any one or a combination of: methyl cellulose, hydroxyethyl cellulose, hydroxypropyl cellulose and hydroxypropyl methylcellulose. The water-soluble cellulose ether may preferably be hydroxyl propyl methylcellulose (HPMC).

In embodiments the first coating may be or comprise hydroxypropyl methyl cellulose and the second coating may be or comprise ethyl cellulose.

The disclosure of the weight gain of the first coating is given as a % by weight of the core. Similarly, the weight gain of the second coating is given as a % by weight of the core, where there is no first coating (sub-coat) on the core. Where the composition comprises a first coating, the weight gain of the second coating is given as a % by weight of the composition that is coated by the second coating, for example the core and the first coating.

The hydrogel forming polymer or the hydrogel forming polymer matrix may be or comprise a hydrocolloid, a non-hydrocolloid gum or chitosan. The hydrogel forming polymer or the hydrogel forming polymer matrix may be a reversible hydrocolloid, for example a thermoreversible hydrocolloid or a thermoreversible hydrogel forming polymer. Alternatively, the hydrogel forming polymer or the hydrogel forming polymer matrix may be or comprise an irreversible hydrocolloid. The hydrogel forming polymer or the hydrogel forming polymer matrix may be or comprise gelatin, agar, a polyethylene glycol, starch, casein, chitosan, soya bean protein, safflower protein, alginates, gellan gum, carrageenan, xanthan gum, phthalated gelatin, succinated gelatin, cellulosephthalate-acetate, oleoresin, polyvinylacetate, polymerisates of acrylic or methacrylic esters and polyvinylacetate-phthalate and any derivative of any of the foregoing; or a mixture of one or more such a hydrogel forming polymers. The hydrogel forming polymer or the hydrogel forming polymer matrix may be or comprise a hydrocolloid selected from carrageenan, gelatin, agar and pectin, or a combination thereof optionally selected from gelatin and agar or a combination thereof, more optionally the hydrogel forming polymer or the or the hydrogel forming polymer matrix forming polymer matrix is or comprises gelatin. The hydrogel forming polymer matrix is or comprises a non-hydrocolloid gum optionally selected from a cross-linked salt of alginic acid. In preferred embodiments the hydrogel forming polymer or the hydrogel forming polymer matrix is or comprises gelatin.

In embodiments the hydrogel forming polymer or the hydrogel forming polymer matrix further comprising a plasticiser, optionally a plasticiser selected from glycerin, a polyol for example sorbitol, polyethylene glycol and triethyl citrate or a mixture thereof, particularly sorbitol.

The hydrogel forming polymer matrix may encapsulate the NFAT inhibitor. The NFAT inhibitor may be encapsulated in solution. The cyclosporin may be in solution or suspended in another component, for example the oil phase or the disperse phase discussed elsewhere, of the composition that is also encapsulated by the hydrogel forming polymer matrix.

The disperse phase may be solid, semi-solid or liquid. In particular, the disperse phase may be liquid. In other particular instances the disperse phase may be semi-solid, for example it may be waxy.

The disperse phase may be or comprise the oil phase, for example the oil phase may be a solid, a semi-solid or a liquid. Suitably the disperse phase or the oil phase is or comprises a liquid lipid and optionally a solvent miscible therewith. The liquid lipid is optionally a medium chain mono- di- or triglyceride (particularly a medium chain triglyceride).

The NFAT inhibitor may be dissolved in the disperse phase. The NFAT inhibitor may be suspended in the disperse phase. The disperse phase may be as described elsewhere herein, for example it may be as described in the immediately preceding two paragraphs.

The oil phase or disperse phase may be or may comprise a liquid lipid. Particularly, the oil phase or disperse phase may comprise or be a short-, medium- or long-chain triglyceride formulation, or a combination thereof, for example a caprylic/capric triglyceride, i.e. a caprylic/capric triglyceride formulation.

Accordingly, in an embodiment the composition comprises cyclosporin, a hydrogel forming polymer matrix, a surfactant and an oil phase comprising a short-, medium- or long-chain triglyceride formulation, or a combination thereof (optionally a caprylic/capric triglyceride, i.e. a caprylic/capric triglyceride formulation) and being dispersed in the hydrogel forming polymer matrix. The composition may be in the form of a dried colloid. The composition may be in the form of a bead.

In a particular embodiment the disperse phase or the oil phase further comprises a solvent, thus optionally the disperse phase or the oil phase may be or comprise a liquid lipid and a solvent. The solvent may be miscible with the liquid lipid and water, optionally wherein the solvent is selected from 2-(2-ethoxyethoxy)ethanol and a poly(ethylene glycol), particularly wherein the solvent is 2-(2-ethoxyethoxy)ethanol. In a further embodiment the disperse phase or oil phase is or comprises a medium chain mono- di- or triglyceride (particularly a medium chain triglyceride), 2-(ethoxyethoxy)ethanol and the surfactant. The disperse phase or oil phase as described in this paragraph may contain the cyclosporin, the cyclosporin may optionally be in solution.

Suitably, the NFAT inhibitor is soluble in the solvent. The solvent may be an alcohol (for example ethanol or isopropanol), a glycol (for example propylene glycol or a polyethylene glycol) or a glycol ether. The solvent may be a glycol ether, for example an ethylene glycol ether, more particularly an alkyl, aryl or aralkyl ethylene glycol ether. The solvent may be a glycol ether selected from 2-methoxyethanol; 2-ethoxyethanol; 2-propoxyethanol; 2-isopropoxyethanol; 2-butoxyethanol; 2-phenoxyethanol; 2-benzyloxyethanol; 2-(2-methoxyethoxy)ethanol; 2-(2-ethoxyethoxy)ethanol; and 2-(2-butoxyethoxy)ethanol. More particularly the solvent is 2-(2-ethoxyethoxy)ethanol or 2-phenoxyethanol. A particular solvent is 2-(2-ethoxyethoxy)ethanol.

Preferably, the oil phase or disperse phase comprises a short-, medium- or long-chain triglyceride formulation, or a combination thereof (optionally a caprylic/capric triglyceride, i.e. a caprylic/capric triglyceride formulation). Where the oil phase or disperse phase comprises a short-, medium- or long-chain triglyceride formulation, or a combination thereof, the triglyceride is substantially all of the disperse phase or oil phase (optionally the liquid lipid). For example, the oil phase or disperse phase may comprise short-, medium- or long-chain triglyceride formulation in an amount of greater than 80% of the oil phase or disperse phase (optionally the liquid lipid), optionally greater than 85%, 90%, 95%, 97%, 98% or 99%. Suitably, the short-, medium- or long-chain triglyceride formulation is substantially free of mono- or di-glycerides. For example, the surfactant may comprise less than 10%, 8%, 5%, 3%, 2% or 1% of a mono- or di-glycerides.

In embodiments the composition further comprises one or more additional surfactants, preferably one additional surfactant. The additional surfactant may be any of the surfactants disclosed herein. The additional surfactant may be referred to as a second surfactant or further surfactant throughout the specification and these terms are used interchangeably.

Suitable surfactants for the second surfactant are described in more detail in the detailed description of the invention. Preferably the second surfactant is an anionic surfactant. For example, the second surfactant may be an alkyl sulphate, for example sodium dodecyl sulphate.

In those embodiments where the liquid composition is in the form of a colloid, the composition is in the form of a solid colloid or the composition comprises a core in the form of a solid colloid, the colloid comprises a continuous phase and a disperse phase, wherein the continuous phase comprises the hydrogel-forming polymer matrix and the second surfactant may be present in the continuous phase, the disperse phase or both. Preferably the second surfactant is present in the continuous phase and the first surfactant is present in the disperse phase. Accordingly, the aqueous phase of the liquid composition may comprise the second surfactant and the oil phase may comprise the first surfactant. In one embodiment the core further comprises one additional surfactant present in at least the continuous phase, the surfactant having an HLB value of greater than 10, for example greater than 20.

The composition may have the characteristics of a composition formed by mixing a disperse phase with a continuous phase to form a colloid, wherein the continuous phase is an aqueous phase comprising hydrogel forming polymer and the disperse phase is an oil phase, wherein the pharmaceutically active ingredient is in the continuous phase or the disperse phase, wherein the colloid is gelled to form the composition. The composition is thus in the form of a solid colloid.

Furthermore, the composition may comprise a core having the characteristics of a core formed by mixing a disperse phase with a continuous phase to form a colloid, wherein the continuous phase is an aqueous phase comprising hydrogel forming polymer and the disperse phase is an oil phase, wherein the pharmaceutically active ingredient is in the continuous phase or the disperse phase, wherein the colloid is gelled to form the core. The core is thus in the form of a solid colloid.

The composition or core comprises cyclosporin, a hydrogel forming polymer matrix, a surfactant and an oil phase and may have the characteristics of a composition obtained by a process comprising:

(i) dissolving a hydrogel-forming polymer in an aqueous liquid to form an aqueous phase solution;
(ii) dissolving the NFAT inhibitor in the oil phase to form a solution;
(iii) mixing the aqueous phase solution (i) and the oil phase solution (ii) to form a colloid (optionally an emulsion);
(iv) ejecting the colloid through a nozzle to form droplets;
(v) causing or allowing the a hydrogel-forming polymer to gel or solidify to form a hydrogel-forming polymer matrix; and
(vi) drying the solid.

The aqueous phase and oil phase may be mixed (for example in step (iii)) in an oil phase to aqueous phase ratio of from 1:4 to 1:10, optionally from 1:4 to 1:8, from 1:5 to 1:7. For example, the oil phase to aqueous phase ratio may be 1:4, 1:5, 1:6 or 1:7.

The oil phase solution (ii) may be prepared by dissolving or dispersing the NFAT inhibitor in a suitable hydrophobic liquid. The hydrophobic liquid may be for example, any of the oils or liquid lipids described herein. By way of example the hydrophobic liquid may be, or comprise, saturated or unsaturated fatty acids or a triglyceride, or an ester or ether thereof with polyethylene glycols. A particular oil for the oil phase is or comprises a triglyceride, for example an oil comprising a medium chain triglyceride, optionally wherein the oil comprises a triglyceride of at least one fatty acid selected from fatty acids having 6, 7, 8, 9, 10, 11 or 12 carbon atoms, e.g. 08-010 fatty acids.

Suitably the aqueous phase solution (i) further comprises an anionic surfactant, e.g. as described elsewhere herein, for example sodium dodecyl sulphate (SDS).

Cores having the characteristics of cores obtained by the above-described processes, for example cores obtained by the processes, may be coated to provide a coating that comprises or is a water-soluble cellulose ether, optionally with a second coating to control or modify release, preferably a polymeric coating as described above and herein. The coated composition may be obtained by applying to the core the coating, e.g. applying to the core first and second coatings as described. Before the coating is applied, the core may be made by a process having steps (i) to (vi) or (i) to (v) described above. Suitable methods for applying the coating(s) are described below and include applying the coatings by spray coating a coating composition onto the core. The processes having steps (i) to (vi) or (i) to (v) themselves form aspects of the invention.

The composition or core may further comprise a second surfactant (also referred to as a further surfactant), optionally wherein the second surfactant is an anionic surfactant, optionally selected from alkyl sulphates, carboxylates or phospholipids, or a non-ionic surfactant, optionally selected from sorbitan-based surfactants, PEG-fatty acids, fatty alcohol ethoxylates, alkylphenol ethoxylate, fatty acid ethoxylates, fatty amide ethoxylates, alkyl glucosides or glyceryl fatty acids, or poloxamers, or a combination thereof. Hence the liquid composition of the invention may comprise at least the following features, an aqueous phase comprising a hydrogel forming polymer, a first surfactant and an oil phase being dispersed in the aqueous phase in which cyclosporin is dissolved and a second surfactant. Similarly, the composition of the invention may comprise at least the following features, cyclosporin, a hydrogel forming polymer matrix, a first surfactant and an oil phase being dispersed in the hydrogel forming polymer matrix and a second surfactant.

In embodiments the second surfactant, as defined above may be the only surfactant in the composition.

In embodiments where the composition is in the form of a solid colloid, the second surfactant may be in the disperse phase or the continuous phase. The second surfactant may be in the continuous phase and may be an anionic surfactant, for example at least one surfactant selected from fatty acid salts and bile salts, particularly an alkyl sulphate, for example sodium dodecyl sulphate. The surfactant in the disperse phase may be a non-ionic surfactant.

In embodiments the composition comprises a second surfactant which is or comprises an anionic surfactant, for example sodium dodecyl sulphate, which is in the continuous phase.

In embodiments the composition further comprises a combination of excipients selected from: an anionic surfactant and a solvent; an anionic surfactant and an oil; and an anionic surfactant, a solvent and an oil. Preferably, the anionic surfactant is an alkyl sulphate, for example sodium dodecyl sulphate, the oil is a medium chain mono-, di- and/or tri-glyceride (optionally a medium chain triglyceride, for example caprylic/capric triglyceride, and the solvent is 2-(ethoxyethoxy)ethanol.

The composition may further comprise an excipient selected from: a surfactant, a solubiliser, a permeability enhancer, a disintegrant, a crystallisation inhibitor, a pH modifier, a stabiliser, or a combination thereof.

The composition of the invention or, where the composition comprises a core, the core may comprise a disperse phase or oil phase, wherein the disperse phase or oil phase is or comprises:

an NFAT inhibitor (for example, cyclosporin);

a medium or long chain fatty acid mono- or di-ester or a combination thereof which does not comprise is not a polyethyleneglycol ether or ester, such as a medium or long chain fatty acid mono- or di-glyceride or a combination thereof, for example glyceryl monooleate/dioleate;

a medium chain mono- di- or tri-glyceride, for example caprylic/capric triglyceride; and

a solvent, for example 2-(ethoxyethoxy)ethanol

and the composition or the core may further comprise a continuous phase or aqueous phase being or comprising:

an anionic surfactant, for example at least one surfactant selected from fatty acid salts and bile salts, particularly an alkyl sulphate, for example sodium dodecyl sulphate

a hydrogel forming polymer matrix which is or comprises a hydrocolloid selected from carrageenan, gelatin, agar and pectin, or a combination thereof optionally selected from gelatin and agar or a combination thereof, more optionally the polymer of the a hydrogel forming polymer matrix is or comprises gelatin; and

optionally a plasticiser, for example a plasticiser selected from glycerin, a polyol for example sorbitol, polyethylene glycol and triethyl citrate or a mixture thereof, particularly sorbitol.

In one embodiment the composition comprises a core and a coating outside the core, wherein the core is in the form of a solid colloid, the colloid comprising a continuous phase and a disperse phase, wherein the disperse phase is or comprises:

an NFAT inhibitor (optionally cyclosporin)

a medium or long chain fatty acid mono- or di-glyceride or a combination thereof which does not comprise is not a polyethyleneglycol ether or ester, for example glyceryl monooleate/dioleate;

a medium chain mono- di- and/or tri-glyceride, for example caprylic/capric triglyceride; and

a co-solvent, for example 2-(ethoxyethoxy)ethanol;

and wherein the continuous phase is or comprises:
a hydrogel-forming polymer matrix which is or comprises a hydrocolloid selected from carrageenan, gelatin, agar and pectin, or a combination thereof optionally selected from gelatin and agar or a combination thereof, more optionally the polymer of the water-soluble polymer matrix is or comprises gelatin;
optionally a plasticiser, optionally a plasticiser selected from glycerin, a polyol for example sorbitol, polyethylene glycol and triethyl citrate or a mixture thereof, particularly sorbitol; and

an anionic surfactant, for example at least one surfactant selected from fatty acid salts and bile salts, particularly an alkyl sulphate, for example sodium dodecyl sulphate;

and wherein the coating on the core is a first coating or a second coating, as described herein.

Suitably the coating comprises a first coating and a second coating outside the first coating; and wherein

the first coating is the coating which is or comprises a water-soluble cellulose ether as described above; and

the second coating is or comprises a coating, suitably a polymeric coating, as defined above to control or modulate release of cyclosporin A from the composition.

In embodiments comprising a first coating and/or a second coating, for example as mentioned in the immediately preceding paragraph, a particular first coating is or comprises hydroxypropylmethyl cellulose and a particular second coating outside the first coating is or comprises a pH independent polymer, for example ethyl cellulose; more particularly the second coating is or comprises ethyl cellulose and optionally a polysaccharide selected from water soluble and naturally occurring polysaccharides, for example pectin or another water-soluble naturally occurring polysaccharide. The second coating may therefore contain pectin or another said polysaccharide or it may be substantially free of pectin and other said polysaccharides. There are therefore disclosed second coatings which comprise ethylcellulose as a controlled release polymer and which further comprise pectin or another said polysaccharide as well as second coatings which comprise ethylcellulose as a controlled release polymer and which do not further comprise pectin or another said polysaccharide.

The hydrogel forming polymer, optionally comprising gelatin, may be present in an amount of 300 to 700 mg/g (optionally 380 to 500 mg/g). The medium chain mono, di and/or tri-glycerides, may be present in an amount of 20 to 200 mg/g (optionally 40 to 80 mg/g). The solvent, for example 2-(ethoxyethoxy)ethanol, may be present in an amount of 100 to 250 mg/g (optionally 160 to 200 mg/g). The medium or long chain fatty acid mono- or di-ester or a combination thereof which does not comprise or is not a polyethyleneglycol ether or ester, for example glyceryl monooleate/dioleate, may be present in an amount of 80 to 200 mg/g (optionally 100 to 150 mg/g). The anionic surfactant, for example sodium dodecyl sulphate, may be present in an amount of up to 100 mg/g or up to 50 mg/g (optionally 15-50 mg/g, preferably 25-50 mg/g or 25-45 mg/g).

The composition or the core may comprise a hydrogel forming polymer comprising gelatin, optionally in an amount of 300 to 700 mg/g, the core further comprising medium chain mono, di and/or tri-glycerides, optionally in an amount of 20 to 200 mg/g, wherein the composition or core further comprises the following components:

solvent, for example 2-(ethoxyethoxy)ethanol, optionally in an amount of 100 to 250 mg/g;

a medium or long chain fatty acid mono- or di-ester or a combination thereof which does not comprise or is not a polyethyleneglycol ether or ester, for example glyceryl monooleate/dioleate, optionally in an amount of 80 to 200 mg/g; and

anionic surfactant, for example sodium dodecyl sulphate, optionally in an amount of up to 50 mg/g.

As will be recognised the composition or core further comprises an NFAT inhibitor (optionally cyclosporin).

The composition or the core may comprise:

a hydrogel forming polymer, for example which is, or comprises, gelatin in an amount of 300 to 700 mg/g;

an NFAT inhibitor (optionally cyclosporin) in an amount of up to about 250 mg/g, for example 50 to 250 mg/g;

medium chain triglycerides, for example Miglyol 810 in an amount of 20 to 200 mg/g, optionally a solvent, for example 2-(ethoxyethoxy)ethanol, which when present is in an amount of 100 to 250 mg/g;

a surfactant comprising a medium or long chain fatty acid mono- or di-ester or a combination thereof which does not comprise or is not a polyethyleneglycol ether or ester, for example glyceryl monooleate/dioleate, in an amount of 80 to 200 mg/g; and

anionic surfactant, for example sodium dodecyl sulphate, in an amount of up to 50 mg/g, for example 10 to 50 mg/g, or optionally 20 to 45 mg/g.

The composition or the core may comprise:

gelatin in an amount of 380-500 mg/g;

cyclosporin in an amount of 90-250 mg/g (optionally 90-200 mg/g or 90-160 mg/g); and

caprylic/capric triglyceride in an amount of 40-80 mg/g;

2-(2-ethoxyethoxy) ethanol in an amount of 160-200 mg/g;

glyceryl monooleate and/or glyceryl dioleate in an amount of 100-150 mg/g; and

SDS in an amount of 15-50 mg/g (optionally 25-50 mg/g or 25-45 mg/g); and

optionally D-sorbitol in an amount of 30-80 mg/g.

The composition or the core may comprise:

gelatin in an amount of 380-500 mg/g;

cyclosporin in an amount of 90-140 mg/g; and

caprylic/capric triglyceride in an amount of 40-80 mg/g;

2-(2-ethoxyethoxy) ethanol in an amount of 160-200 mg/g;

glyceryl monooleate and/or glyceryl dioleate in an amount of 100-150 mg/g; and

SDS in an amount of 15-50 mg/g (optionally 25-50 mg/g or 25-45 mg/g); and

optionally D-sorbitol in an amount of 30-80 mg/g.

The composition or core may be a colloid. Where the composition or the core is a colloid, the NFAT inhibitor, for example cyclosporin, may be dissolved in the disperse phase of the colloid.

The composition or core may be a colloid; thus, the composition or core may comprise a continuous phase and a disperse phase wherein the continuous phase comprises:

gelatin in an amount of 380-500 mg/g; and

optionally D-sorbitol in an amount of 30-80 mg/g;

the disperse phase comprises:

cyclosporin in an amount of 90-140 mg/g; and

caprylic/capric triglyceride in an amount of 40-80 mg/g;

and the composition further comprises:

2-(2-ethoxyethoxy)ethanol in an amount of 160-200 mg/g;

glyceryl monooleate and/or glyceryl dioleate in an amount of 100-150 mg/g; and

SDS in an amount of 15-50 mg/g.

A colloidal composition or core comprising a continuous phase comprising:

a hydrogel forming polymer matrix comprising gelatin in an amount of 300 to 700 mg/g;

a disperse phase comprising:

cyclosporin in an amount of up to 200 mg/g; and

a medium chain tri-glyceride in an amount of 20 to 200 mg/g;

and the composition further comprising:

solvent in an amount of 100 to 250 mg/g;

surfactant (a first surfactant) being or comprising a medium or long chain fatty acid mono- or di-ester or a combination thereof which does not comprise is not a polyethyleneglycol ether or ester, for example glyceryl monooleate/dioleate; and

anionic surfactant (a second surfactant) in an amount of up to 50 mg/g.

In the embodiments above which refer to mg/g of a component, the concentration is based upon the dry weight of the composition.

Suitably in the six compositions or cores described immediately above, the composition is a colloid comprising a disperse phase and a continuous phase; wherein the disperse phase comprises cyclosporin, medium-chain triglyceride and medium or long chain fatty acid mono- or di-ester surfactant; and the continuous phase comprises the hydrogel forming polymer (e.g. gelatin) and an anionic surfactant (e.g. SDS).

The invention includes within its scope compositions wherein the core is a colloid having a disperse phase and the continuous phase (matrix phase) of the colloid further includes dispersed particles of a pharmaceutically active ingredient, for example microparticles or nanoparticles. The disperse phase and continuous phase may otherwise be as described elsewhere in this specification.

The composition of the invention and/or the core may be in the form of a minibead. It may be that the core is a minibead and the first coating and, where applicable, the second coating in conjunction with the core are in the form of a minibead. However, it may be possible for the core to be a minibead and the composition not to be a minibead. The composition may additionally comprise a multiplicity of minibeads. Hence the invention contemplates a minibead with the features of the pharmaceutical compositions disclosed herein.

The composition or the minibead may have a largest cross sectional dimension of a core of from about 0.01 mm to about 5 mm, for example from 1 mm to 5 mm, as in the case of from 1 mm to 3 mm or 1 mm to 2 mm. The minibead may be spheroidal. The spheroidal minibeads may have an aspect ratio of no more than 1.5, for example from 1.1 to 1.5.

The composition of the invention may be for oral administration. The composition may be formulated into a unit dosage form for oral administration comprising from 0.1 mg to 1000 mg, optionally from 1 mg to 500 mg, for example 10 mg to 300 mg, or 25 to 250 mg suitably about 25 mg, about 35 mg, about 37.5 mg, about 75 mg, about 150 mg, about 180 mg, about 210 mg, about 250 mg or about 300 mg of an NFAT inhibitor (optionally cyclosporin). Suitably the composition is in a multiple minibead unit dosage form selected from multiple minibeads in, for example, soft or hard gel capsules, gelatin capsules, HPMC capsules, compressed tablets or sachets. The minibeads may be as described elsewhere herein.

According to a further feature of the invention there is provided a composition comprising an NFAT inhibitor and a surfactant, optionally wherein the surfactant comprises, or is a medium chain or long chain fatty acid mono- or di-glyceride or a combination thereof. Suitably the composition does not comprise or is not a polyethyleneglycol ether or ester. Suitably the surfactant is present in an amount of at least 6% by weight of the composition, for example at least 10%, at least 15% or at least 20% by weight of the composition. Optionally the surfactant is present in an amount of from 10 to about 50% by weight.

The composition according to this aspect of the invention may further comprise an oil phase, for example any of the oil phases described herein.

The NFAT inhibitor (preferably cyclosporin) may be partially or completely dissolved in the composition. Suitably the NFAT inhibitor (preferably cyclosporin) is completely dissolved in the composition.

A particular composition comprises:
(i) 10 to 60 parts NFAT inhibitor (preferably cyclosporin A);
(ii) 5 to 40 parts of a medium chain fatty acid triglyceride, for example a caprylic/capric triglyceride;
(iii) 10 to 50 parts of the surfactant; and
(iv) 0 to 60 parts solvent;
   wherein all parts are parts by weight and the sum of the parts (i)+(ii)+(iii)+(iv)=100.

Another composition comprises:
(i) 10 to 40 parts cyclosporin A;
(ii) 5 to 25 parts of a medium chain fatty acid triglyceride, a caprylic/capric triglyceride;
(iii) 15 to 30 parts of the surfactant; and
(iv) 10 to 60 parts solvent (optionally 20 to 40 parts or 25-30 parts solvent), for example 2-(2-ethoxyethoxy)ethanol);
   wherein all parts are parts by weight and the sum of the parts (i)+(ii)+(iii)+(iv)=100.

Optionally in this aspect the surfactant is selected from glyceryl caprylate, glyceryl caprate, glyceryl monooleate, glyceryl dioleate and glycerol monolinoleate, or a combination thereof.

The invention additionally provides a method for administering an NFAT inhibitor to a subject, comprising administering to the gastrointestinal tract of a subject (optionally orally) a composition described herein. The method may be performed to reduce or prevent undesirable effects occurring in conjunction with a therapy mediated by NFAT activated T cells. The undesirable effects may be selected from cytokine release syndrome (CRS) and symptoms associated with inflammatory bowel diseases. The undesirable effects may be reduced or prevented with maintenance of effectiveness of therapy. The composition may be any composition described herein.

A further aspect of the invention provides the use of a composition described herein for use in the manufacture of a medicament for reducing or preventing one or more undesirable effects occurring in conjunction with a therapy mediated by NFAT-activated T cells, wherein said undesirable effects are selected from Cytokine Release Syndrome (CRS) and symptoms associated with inflammatory bowel diseases and wherein said composition is administered to the gastrointestinal tract whereby said one or more undesirable effects are reduced or prevented with maintenance of effectiveness of said therapy.

In an aspect of the invention there is provided a process for making a composition, the process comprising mixing an oil phase with an aqueous phase comprising a hydrogel forming polymer, wherein the oil phase has an NFAT inhibitor (optionally cyclosporin) in solution and comprises a surfactant, the process further comprising the step of causing the emulsion to solidify

The composition of the invention may be a composition with the characteristics of a composition obtained by the process described herein.

Optionally, the oil phase and the aqueous phase are mixed in an oil phase to aqueous phase ratio of from 1:2 to 1:12, optionally 1:4 to 1:10, 1:4 to 1:8, for example 1:5 or 1:7.

The process may further comprise the step of:

coating a core with a coating comprising HPMC wherein the weight gain due to the coating is from 0.5% to 20% of the weight of the pharmaceutical composition. The core may comprise a pharmaceutically active ingredient and may be a core as described in this specification.

For certain active ingredients it may be desirable to limit or delay release of the active from the composition until the composition has passed through the stomach and upper GI tract. The compositions of the invention comprising a second coat may be particularly suitable for such applications. The second coat acts to delay release from the composition, whilst the presence of the first coating (e.g. HPMC) increases the amount of active released when the composition releases the active in the lower GI tract. The period of delay to the release of the active as a result of the presence of the second coating can be tailored by appropriate selection of the nature or amount of second coating used. For a given second coating material a higher weight gain of coating will generally increase the time period between administration of the composition and release of the active. The compositions of the invention can therefore be used to provide high levels of release of active agent at very specific parts of the GI tract to provide. Such delayed release compositions may be particularly beneficial when the active has undesirable side effects which may arise from systemic absorption higher in the GI tract.

Included in this description by reference are the subject matters of the appended claims. The description is therefore to be read together with the claims and features mentioned in the claims are applicable to the subject matters of the description. For example, a feature described in a process claim is applicable also to products mentioned in the description, where the feature is manifested in the product. For example, a feature mentioned in a product claim is applicable also to relevant process subject matters contained in this description. Similarly, a feature mentioned in the description in the context of a process is applicable also to products mentioned in the description, where the feature is manifested in the product. Also, a feature mentioned in the description in the context of a product is applicable also to relevant process subject matters contained in this description.

### BRIEF DESCRIPTION OF THE DRAWINGS

Embodiments of the invention are further described hereinafter with reference to the accompanying drawings, in which:

FIG. 1 is a graph showing mouse survival.

FIG. 2 is a graph showing weight change over time in each mouse group of the study.

FIGS. 3a-l are bar charts showing data relating to T cell levels in the spleen, lungs, liver and gut of the mice.

FIGS. 4a-f are bar charts for cytokine levels in the colon of mice from each group.

FIGS. 5a-e are bar charts for cytokine levels in the small intestine of mice from each group.

FIGS. 6a-c are bar charts for cytokine levels in the spleen of mice from each group.

FIGS. 7a-e are bar charts for cytokine levels in the lung of mice from each group.

FIG. 8 is a bar chart of FoxP3+ cells in the GI.

FIG. 9 is a bar chart of TNFα producing T cells in the GI.

FIG. 10 shows histological slides of the small intestine of mice from each test group.

FIG. 11 histological slides of the small intestine of mice from each test group stained to show the amount of apoptosis.

### DETAILED DESCRIPTION

As indicated above, there are a growing number of therapies which rely on provision or promotion of NFAT-activated T cells to target disease antigens, especially tumour antigens. The invention may find use whenever such activated T cells are employed for intended therapeutic benefit. The NFAT activated T cell may be any NFAT activated T-cell including, for example NFAT activated Natural Killer T-cells (NK-T cells). It is envisaged, however, that its use may be particularly favoured where a pre-conditioning regime, employing chemotherapy and/or radiotherapy, is employed to deplete autologous T cells. In such instances, it may be preferred to administer a composition in accordance with the invention throughout the pre-conditioning regime or at least ahead of the application of the activated therapeutic T cells. As indicated above, such administration may also desirably continue during the actual cell therapy period and in instances where the therapy is directed against a haematological malignancy may be continued during following application of allo-HCT. Examples of T cell therapies for which application of the invention may be advantageous are described above.

Preferably the NFAT inhibitor of the invention is cyclosporin. Reference to active ingredient herein is reference to an NFAT inhibitor. The two terms are used interchangeably. Reference to “cyclosporin” herein is a reference to cyclosporin-A (also known as cyclosporine and the INN ciclosporin. It is contemplated that other forms of cyclosporin may be used in the compositions described herein, for example cyclosporin-B, -C, -D or -G and derivatives or pro drugs of any thereof.

“Cell therapy”, also called “cellular therapy” or “cytotherapy” is therapy in which cellular material is administered to a patient or wherein the administration of the therapy to a patient elicits an NFAT activated T-cell response, for example through direct administration of a T-cell-based therapy, for example a T-cell therapy described herein. Administration is ordinarily carried out by injection into a patient. The cellular material may be generally intact, living cells. For example, a cell therapy includes T cells, modified T cells capable of fighting cancer cells via cell-mediated immunity may be injected in the course of immunotherapy.

Maintenance of effectiveness of the therapy mediated by NFAT activated T cells can be regarded as the therapeutic outcome of the therapy being positive when the composition comprising the inhibitor of NFAT is used in conjunction with the therapy. Accordingly, the composition comprising the inhibitor of NFAT does not adversely affect the desired therapeutic effects of the therapy on the condition being treated by the therapy, for example a cancer. The positive outcome of the therapy can be readily identified by a doctor, potentially determined based on the same scoring system or ranking that resulted in the decision to begin therapy. For example, prior to treatment with the therapy mediated by NFAT activated T cells, the patient may have cancer defined at stage: T4 N3 M1, T4 N3 M0, T4 N2 M1, T4 N1 M1, T4 N2 M0, T4 N1 M0, T3 N3 M1, T3 N3 M0, T3 N2 M1, T3 N1 M1, T3 N2 M0, T3 N1 M0, T2 N3 M1, T2 N3 M0, T2 N2 M1, T2 N1 M1, T2 N2 M0 or T2 N1 M0; in order for maintenance of effectiveness of the therapy the patients cancer may be categorised at a lower stage following treatment with the therapy in conjunction with the composition comprising the inhibitor of NFAT. For example, the patient may have been categorised as a T4 N3 M1 prior to treatment and after therapy with adjunct administration of the composition of the invention might be categorised as T3 N2 M1. The term “maintenance of effectiveness of the therapy” includes the following and combinations thereof: the therapy accomplishing remission or response; T cell levels (optionally CART cell levels) in serum remaining at efficacious levels; serum T cell levels remaining at about the level before administration of the composition of the invention; and serum T cell levels dropping by at most 50% of levels before administration of the composition of the invention. Also “maintenance of therapy” may include: maintenance of remission, e.g. in the case of ALL; % blast in bone marrow of <5%, <10%, <20%, or <30%. In the case of solid tumours “maintenance of the therapy” may include absence of primary or secondary tumours, lack of metastases or inhibition of tumour growth. “Maintenance of the therapy” may also, or alternatively, refer to the prevention of metastases, the prevention of micrometastases, or the reduction or absence of increase of metastatic cells.

The “therapy mediated by NFAT activated T cells” may provide the following effects and combinations thereof when used to treat a patient: (1) reducing the risk of or inhibiting, e.g. delaying, initiation and/or progression of, a state, disorder or condition; (2) preventing, e.g. reducing the risk of, or delaying the appearance of clinical symptoms of a state, disorder or condition developing in a patient (e.g. human or animal) that may be afflicted with or predisposed to the state, disorder or condition but does not yet experience or display clinical or subclinical symptoms of the state, disorder or condition; (3) inhibiting the state, disorder or condition (e.g., arresting, reducing or delaying the development of the disease, or a relapse thereof in case of maintenance treatment, of at least one clinical or subclinical symptom thereof); and/or (4) relieving the condition (e.g. causing regression of the state, disorder or condition or at least one of its clinical or subclinical symptoms). Where the composition of the invention is used in the treatment of a patient, for example as an adjunct treatment with the therapy, treatment contemplates any one or more of: maintaining the health of the patient; restoring or improving the health of the patient; and delaying the progression of the undesirable effect. The benefit to a patient to be treated may be either statistically significant or at least perceptible to the patient or to the physician. It will be understood that a medicament will not necessarily produce a clinical effect in every patient to whom it is administered, and this paragraph is to be understood accordingly. The compositions and methods described herein are of use for therapy and/or prophylaxis of an undesirable effect of a therapy mediated by NFAT activated T cells.

The therapy mediated by NFAT activated T cells may be for the treatment of for example cancer. However, other conditions wherein NFAT activated T cells may be beneficial are envisioned. Cancer may be a solid tumour or a blood cancer. Cancer may be sarcomas, melanomas, skin cancers, haematological malignancies, haematological tumours, lymphoma, carcinoma or leukaemia, Cancer may be acute lymphoblastic leukaemia, B cell acute lymphoblastic leukaemia (B-ALL), chronic lymphocytic leukaemia (CLL), acute myelogenous leukaemia (AML), B-cell malignancy, B-cell lymphoma, diffuse large B cell lymphoma, chronic lymphocyte leukaemia, non-Hodgkin lymphoma for example ABC-DLBCL, mantle cell lymphoma, follicular lymphoma, hairy cell leukaemia B-cell non-Hodgkin lymphoma, Waldenstrom's macroglobulinemia, multiple myeloma, bone cancer, bone metastasis, immunosuppression melanoma, metastatic non-small cell lung cancer, non-small cell lung cancer, metastatic melanoma, brain tumour, hormone refractory prostate cancer, prostate cancer, metastatic breast cancer, breast cancer, stage IV melanoma, neuroblastoma solid tumour, metastatic pancreatic cancer, pancreatic cancer, myelodisplastic syndrome, ovarian cancer, fallopian tube cancer, peritoneal tumour, colorectal cancer, lung cancer, cervical cancer, testicular cancer, renal cancer or cancer of the head and neck, In an embodiment the cancer is not a cancer of the gastrointestinal tract, more particularly in this embodiment the cancer is not a cancer of the lower GI tract, for example, the cancer is not colorectal cancer.

Accordingly the cancer may be a haematological cancer for example lymphoma, chronic lymphocytic leukaemia (CLL), acute lymphoblastic leukaemia (ALL) or \ non-Hodgkin lymphoma. The cancer may be a solid cancer for example breast, ovarian, pancreatic, colon, gastric, lung or prostate cancer or melanoma. The cancer may be a melanoma.

The therapy may be a therapy targeting metastatic cells in lymphatic tissue, for example the lymphatic tissue of the GIT or lymph nodes.

Optionally, cancer treated by the therapy is B cell acute lymphoblastic leukaemia (B-ALL), chronic lymphocytic leukaemia (CLL) or acute myelogenous leukaemia (AML).

The composition comprising an inhibitor of NFAT may be used in the reduction or prevention of undesirable effects associated with the therapy mediated by NFAT activated T cells described herein and this may include a maintenance therapy of patients who have been administered with a therapy mediated by NFAT activated T cells and suffered undesirable effects whose condition has subsequently improved, e.g. because of treatment with therapy. Such patients may or may not suffer a symptomatic disorder. Maintenance therapy using a composition comprising an inhibitor of NFAT aims to arrest, reduce or delay (re-)occurrence or progression of undesirable effects associated with the therapy mediated by NFAT activated T cells.

The composition comprising an inhibitor of NFAT may be administered simultaneously, sequentially or separately with the therapy mediated by NFAT activated T cells so as to provide the desired reduction or prevention of undesirable effects associated with the therapy. For example, the composition may be administered prior to or substantially simultaneously with the therapy such that the NFAT inhibitor is present in the GI tract, for example the colon, when the therapy is present in the patient. Alternatively, the composition may be administered to the patent after administration of the therapy to the patient. The undesirable effects associated with the therapy may not be observed for some time after the therapy has been administered. Accordingly, the composition comprising the NFAT inhibitor may be used after the administration of the therapy in response to the onset of an undesirable effect of the therapy so as to ameliorate and reduce the effect for example the onset of CRS. Accordingly, the composition may be used as a rescue therapy to counter an undesirable effect of the therapy after it has occurred. The composition may also be used prophylactically to prevent or reduce the risk of occurrence of an undesirable effect of the therapy such as CRS. As described herein the composition delivers the NFAT inhibitor to the GI tract, for example the lower GI tract, and suitably mediates the undesirable effects associated with the therapy such as CRS, whilst maintaining the therapeutic effectiveness of the said therapy against the condition being treated, for example a cancer.

“Effective amount” means an amount sufficient to achieve the desired treatment, e.g. reduce or prevent one or more undesirable effects and maintain effectiveness of a therapy or result in the desired therapeutic or prophylactic response. The therapeutic or prophylactic response can be any response that a user (e.g., a clinician) will recognise as an effective response to the therapy. It is further within the skill of one of ordinary skill in the art to determine appropriate treatment duration, appropriate doses, and any potential combination treatments, based upon an evaluation of therapeutic or prophylactic response.

The terms “dry” and “dried” as applied to compositions or compositions of the disclosure may each include reference to compositions or compositions containing less than 5% free water by weight, e.g. less than 1% free water by weight. Primarily, however, “dry” and “dried” as applied to compositions of the disclosure mean that the hydrogel present in the initial solidified composition has dried sufficiently to form a rigid composition. Where a solid colloid is referred to this also refers to a dried colloid according to the definition herein.

Ingredients and excipients of the described compositions are suitable for the intended purpose. For example, pharmaceutical compositions comprise pharmaceutically acceptable ingredients.

If not otherwise stated, ingredients, components, excipients etc. of the compositions of the invention are suitable for one or more of the intended purposes discussed elsewhere herein.

For the avoidance of doubt, it is hereby stated that the information disclosed earlier in this specification under the heading “Background” is relevant to the invention and is to be read as part of the disclosure of the invention.

Where the invention is referred to as a formulation it takes the same meaning as the composition of the invention. Accordingly, formulation and composition are used interchangeably.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

### Composition

In an embodiment the composition comprises a matrix and an NFAT inhibitor. The matrix may be formed with a hydrogel-forming polymer, and may contain additional excipient(s) to the polymer. The active ingredient is contained within the matrix. The active ingredient may be in solution or in suspension, or in a combination thereof; however the invention is not limited to compositions comprising a solution or suspension of the active and it includes, for example, active ingredients encapsulated in liposomes or cyclodextrin. The matrix may contain inclusions in which the active ingredient is comprised; for example, the inclusions may comprise a hydrophobic medium in which the active ingredient is dissolved or suspended. An active ingredient may therefore be directly dissolved or suspended in the matrix, or it may be dissolved or suspended indirectly in the matrix by way of inclusions in which the active ingredient is dissolved or suspended.

The composition, therefore, comprises a matrix-forming polymer, in particular a hydrogel-forming polymer. The matrix of the composition may be or comprise a polymer matrix comprising a polymer selected from a water-permeable polymer, a water-swellable polymer and a biodegradable polymer. In particular, the matrix is or comprises a hydrogel-forming polymer described in more detail below.

Modified release of the active ingredient from the composition may be achieved by virtue of the properties of the matrix material. For example the matrix may be a permeable or erodible polymer within which the active ingredient is contained, e.g. dissolved or suspended; following oral administration the matrix is gradually dissolved or eroded thereby releasing the active ingredient from the matrix. Erosion may be achieved by biodegradation of a biodegradable polymer matrix. Where the matrix is permeable, water permeates the matrix enabling the drug to diffuse from the matrix. A matrix formed with a hydrogel-forming polymer may therefore include a modified release polymer. As such modified release polymers may be mentioned cellulose derivatives, for example hydroxypropylmethyl cellulose, poly(lactic acid), poly(glycoloic)acid, poly(lactic-co glycolic acid copolymers), polyethylene glycol block co-polymers, polyorthoesters, polyanhydrides, polyanhydride esters, polyanhydride imides, polyamides and polyphosphazines.

### Polymer Matrix

The composition of the invention may comprise an NFAT inhibitor (e.g. cyclosporin), a hydrogel forming polymer matrix, a surfactant and an oil phase being dispersed in the hydrogel forming polymer matrix. In addition, in certain embodiments of the invention the composition of the invention comprises a core wherein the core comprises an NFAT inhibitor (e.g. cyclosporin), a hydrogel forming polymer matrix, a surfactant and an oil phase being dispersed in the hydrogel forming polymer matrix. The composition or the core comprises a continuous phase or matrix phase, which may be or comprise the hydrogel forming polymer matrix, to provide mechanical strength. In embodiments the cyclosporin is comprised within a disperse phase or oil phase within the continuous phase or matrix. The NFAT inhibitor (e.g. cyclosporin) may be present as a disperse phase within the hydrogel-forming polymer matrix (continuous phase or aqueous phase) of the core or composition. The disperse phase may be or comprise the oil phase. For example the disperse phase may comprise a lipid and an NFAT inhibitor (e.g. cyclosporin). The core or the composition may be prepared by dispersing the NFAT inhibitor (e.g. cyclosporin), dissolved in the oil phase within an aqueous phase comprising the hydrogel forming polymer matrix to form a colloid and then causing the composition to solidify (gel), thereby immobilising the cyclosporin within the hydrogel-forming polymer matrix.

The core may have the form of a solid colloid, the colloid comprising a continuous phase and a disperse phase, wherein the continuous phase is or comprises the hydrogel-forming polymer matrix and the disperse phase is or comprises an oil phase optionally comprising the NFAT inhibitor (e.g. cyclosporin). The disperse phase may comprise a vehicle containing the NFAT inhibitor (e.g. cyclosporin), for example containing it as a solution or a suspension or a combination of both. The vehicle may be an oil phase as described herein.

Such cores comprising a hydrogel-forming polymer and a disperse phase comprising an NFAT inhibitor (e.g. cyclosporin) are described in more detail below.

### Delayed Release Coatings

In certain embodiments the invention provides compositions having a coating that comprises, or is, a coating-forming polymer, wherein the coating-forming polymer is a hydrogel-forming polymer; the coating may be a first coating outside which is a second coating. The second coating may be a delayed release coating, although the invention does not require that the second coating be a delayed release coating. The second coating may comprise or be a delayed release polymer.

The first coating may be present in an amount described elsewhere in this specification.

The first coating may be present in an amount corresponding to a weight gain due to the first coating of from 0.5% to 20% by weight of the core.

Furthermore, the composition may comprise a first coating present in an amount corresponding to a weight gain due to the coating selected from ranges of from: 0.5% to 15%; 1% to 15%; 1% to 12%; 1% to 10%; 1% to 8%; 1% to 6%; 1% to 4%, 2% to 10%; 2% to 8%; 2% to 6%; 2% to 7%; 2% to 4%; 4% to 8%; 4% to 7%, 4% to 6%, 5% to 7%; 7% to 20%; 7% to 16%; 9% to 20%; 9% to 16%; 10% to 15%; and 12% to 16%.

The invention provides for a pharmaceutical composition comprising a core, a first coating and a second coating outside of the first coating, wherein the core comprises an NFAT inhibitor (e.g. cyclosporin), a hydrogel forming polymer matrix, a surfactant and an oil phase being dispersed in the hydrogel forming polymer matrix, the first coating comprises or is a water soluble cellulose ether, and the second coating comprises or is a delayed release polymer, and the first coating may be present in an amount corresponding to a weight gain due to the first coating of from 0.5% to 20% by weight of the core, wherein the surfactant is a medium chain or long chain fatty acid mono- or di-glyceride or a combination thereof and does not comprise or is not a polyethyleneglycol ether or ester.

The composition of the invention may comprise a first coating with a thickness of 1 μm to 1 mm. Thus, the % weight gain due to the coating specified above may correspond to a thickness of 1 μm to 1 mm.

The first coating may have a thickness selected from ranges of from: 1 μm to 500 μm; 10 μm to 250 μm; 10 μm to 100 μm; 10 μm to 50 μm; 10 μm to 20 μm; 50 μm to 100 μm; 100 μm to 250 μm; 100 μm to 500 μm; 50 μm to 500 μm; 50 μm to 250 μm; 100 μm to 1 mm; 500 μm to 1 mm. The coating having the thicknesses disclosed in this paragraph may be any of the coatings in the application. In particular the coating referred to in this paragraph may be the water-soluble cellulose ether coating.

The first coating may be present in a weight gain selected from a range of from: 1% to 20%, 4% to 7%, 5% to 7%, 4% to 15%, 8% to 15%, 4% to 12% and 8% to 12%. The second coating may be present in a weight gain selected from a range of from: 8% to 15% or 8% to 12%.

In addition, the invention provides for a pharmaceutical composition comprising a core, a first coating and a second coating outside of the first coating, wherein the core comprises an NFAT inhibitor (e.g. cyclosporin), a hydrogel forming polymer matrix, a surfactant and an oil phase being dispersed in the hydrogel forming polymer matrix, the first coating comprises or is a water soluble cellulose ether, and the second coating comprises or is a delayed release polymer, and the first coating has a thickness of from 1 μm to 1 mm.

The second coating may be present in an amount described elsewhere herein. Suitably the second coating provides a coating thickness on the composition of from about 10 μm to about 1 mm, for example, from about 10 μm to about 500 μm, from about 50 μm to about 1 mm, or about from about 50 μm to about 500 μm. The thickness may therefore be from about 100 μm to about 1 mm, e.g. 100 μm to about 750 μm or about 100 μm to about 500 μm. The thickness may be from about 250 μm to about 1 mm, e.g. about 250 μm to about 750 μm or 250 μm to about 500 μm. The thickness may be from about 500 μm to about 1 mm, e.g. about 750 μm to about 1 mm or about 500 μm to about 750 μm. The thickness may therefore be from about 10 μm to about 100 μm, e.g. from about 10 μm to about 50 μm or about 50 μm to about 100 μm.

It is contemplated within any aspect or embodiment where there is a second coating (also referred to as an outer coating) that the second coating may be present in a % weight gain of from 2% to 40%. In addition the second coating may be present in an amount corresponding to a weight gain due to the coating selected from ranges of from: 4% to 30%, 4% to 7%, 7% to 40%, 7% to 30%, 8% to 25%, 8% to 20%, 2% to 25%, 2% to 20%, 4% to 25%, 4% to 20%, 4% to 15%, 4% to 13%, 7% to 15%, 7% to 13%, 8% to 12%, 9% to 12% and 20% to 25%.

In any aspect and embodiment of the invention the first coating may be present in a % weight gain relative to the core of from 0.5% to 20%, preferably 1% to 16% or 4% to 16%, and the second coating may be present in a % weight gain of 4% to 24%, 7% to 24%, 22% to 24%, 7% to 15%, or 8% to 12%, preferably 22% to 24%, 7% to 15%, or 8% to 12%.

The core is preferably in the form of a minibead, for example as described hereafter in more detail, for example in the form of a solid colloid. The second coat may be a film or it may be a membrane. The second coat, e.g. film or membrane, may serve to delay release until after the stomach; the coat may therefore be an enteric coat. The delayed release coat may comprise one or more delayed release substances, preferably of a polymeric nature (e.g. methacrylates etc; polysaccharides etc as described in more detail below), or combination of more than one such substance, optionally including other excipients, for example, plasticizers. Preferred plasticizers, if they are used, include hydrophilic plasticizers for example triethyl citrate (TEC) which is particularly preferred when using the Eudragit® family of polymers as coatings as described below. Another preferred plasticiser, described in more detail below in relation to coating with ethyl cellulose, is dibutyl sebacate (DBS). Alternative or additional optionally included excipients are glidants. A glidant is a substance that is added to a powder or other medium to improve its flowability. A typical glidant is talc which is preferred when using the Eudragit® family of polymers as coatings.

The delayed release coating (the second coating) may be applied as described below and may vary as to thickness and density. The amount of coat is defined by the additional weight added to (gained by) the dry composition (e.g. the core) to which it is applied. Weight gain is preferably in the range 0.1% to 50%, preferably from 1% to 15% of the dry weight of the core, more preferably in the range 3% to 10% or in the range 5-12% or in the range 8-12%.

Polymeric coating material of a delayed release coating may comprise methacrylic acid co-polymers, ammonio methacrylate co-polymers, or mixtures thereof. Methacrylic acid co-polymers such as, for example, EUDRAGIT™ S and EUDRAGIT™ L (Evonik) are particularly suitable. These polymers are gastroresistant and enterosoluble polymers. Their polymer films are insoluble in pure water and diluted acids. They may dissolve at higher pHs, depending on their content of carboxylic acid. EUDRAGIT™ S and EUDRAGIT™ L can be used as single components in the polymer coating or in combination in any ratio. By using a combination of the polymers, the polymeric material can exhibit solubility at a variety of pH levels, e.g. between the pHs at which EUDRAGIT™ L and EUDRAGIT™ S are separately soluble. In particular, the coating may be an enteric coating comprising one or more co-polymers described in this paragraph. A particular coating material to be mentioned is Eudragit L 30 D-55.

The trade mark “EUDRAGIT” is used hereinafter to refer to methacrylic acid copolymers, in particular those sold under the trade mark EUDRAGIT by Evonik.

The delayed release coating, where present, can comprise a polymeric material comprising a major proportion (e.g., greater than 50% of the total polymeric coating content) of at least one pharmaceutically acceptable water-soluble polymer, and optionally a minor proportion (e.g., less than 50% of the total polymeric content) of at least one pharmaceutically acceptable water insoluble polymer. Alternatively, the membrane coating can comprise a polymeric material comprising a major proportion (e.g., greater than 50% of the total polymeric content) of at least one pharmaceutically acceptable water insoluble polymer, and optionally a minor proportion (e.g., less than 50% of the total polymeric content) of at least one pharmaceutically acceptable water-soluble polymer.

Ammonio methacrylate co-polymers such as, for example, EUDRAGIT™ RS and EUDRAGIT™ RL (Evonik) are suitable for use in the present invention. These polymers are insoluble in pure water, dilute acids, buffer solutions, and/or digestive fluids over the entire physiological pH range. The polymers swell in water and digestive fluids independently of pH. In the swollen state, they are then permeable to water and dissolved active agents. The permeability of the polymers depends on the ratio of ethylacrylate (EA), methyl methacrylate (MMA), and trimethylammonioethyl methacrylate chloride (TAMCl) groups in the polymer. For example, those polymers having EA:MMA:TAMCl ratios of 1:2:0.2 (EUDRAGIT™ RL) are more permeable than those with ratios of 1:2:0.1 (EUDRAGIT™ RS). Polymers of EUDRAGIT™ RL are insoluble polymers of high permeability. Polymers of EUDRAGIT™ RS are insoluble films of low permeability. A diffusion-controlled pH-independent polymer in this family is RS 30 D which is a copolymer of ethyl acrylate, methyl methacrylate and a low content of methacrylic acid ester with quaternary ammonium groups present as salts to make the polymer permeable. RS 30 D is available as an aqueous dispersion.

The amino methacrylate co-polymers can be combined in any desired ratio, and the ratio can be modified to modify the rate of drug release. For example, a ratio of EUDRAGIT™ RS:EUDRAGIT™ RL of 90:10 can be used. Alternatively, the ratio of EUDRAGIT™ RS:EUDRAGIT™ RL can be about 100:0 to about 80:20, or about 100:0 to about 90:10, or any ratio in between. In such compositions, the less permeable polymer EUDRAGIT™ RS generally comprises the majority of the polymeric material with the more soluble RL, when it dissolves, permitting gaps to be formed through which solutes can come into contact with the core allowing for the active to escape in a controlled manner.

The amino methacrylate co-polymers can be combined with the methacrylic acid co-polymers within the polymeric material in order to achieve the desired delay in the release of the drug and/or poration of the coating and/or exposure of the composition within the coating to allow egress of drug and/or dissolution of the immobilization or water-soluble polymer matrix. Ratios of ammonio methacrylate co-polymer (e.g., EUDRAGIT™ RS) to methacrylic acid co-polymer in the range of about 99:1 to about 20:80 can be used. The two types of polymers can also be combined into the same polymeric material, or provided as separate coats that are applied to the beads.

Eudragit™ FS 30 D is an anionic aqueous-based acrylic polymeric dispersion consisting of methacrylic acid, methyl acrylate, and methyl methacrylate and is pH sensitive. This polymer contains fewer carboxyl groups and thus dissolves at a higher pH (>6.5). The advantage of such a system is that it can be easily manufactured on a large scale in a reasonable processing time using conventional powder layering and fluidized bed coating techniques. A further example is EUDRAGIT® L 30D-55 which is an aqueous dispersion of anionic polymers with methacrylic acid as a functional group. It is available as a 30% aqueous dispersion.

In addition to the EUDRAGIT™ polymers described above, a number of other such copolymers can be used to control drug release. These include methacrylate ester co-polymers such as, for example, the EUDRAGIT™ NE and EUDRAGIT™ NM ranges. Further information on the EUDRAGIT™ polymers can be found in “Chemistry and Application Properties of Polymethacrylate Coating Systems,” in Aqueous Polymeric Coatings for Pharmaceutical Dosage Forms, ed. James McGinity, Marcel Dekker Inc., New York, pg 109-114 the entirety of which is incorporated herein by reference.

Several derivatives of hydroxypropyl methylcellulose (HPMC) also exhibit pH dependent solubility and may be used in the invention for the delayed release coating. As examples of such derivatives may be mentioned HPMC esters, for example hydroxypropyl methylcellulose phthalate (HPMCP), which rapidly dissolves in the upper intestinal tract and hydroxypropyl methylcellulose acetate succinate (HPMCAS) in which the presence of ionisable carboxyl groups causes the polymer to solubilize at high pH (>5.5 for the LF grade and >6.8 for the HF grade). These polymers are commercially available from Shin-Etsu Chemical Co. Ltd. As with other polymers described herein as useful for delayed release coatings, HPMC and derivatives (e.g. esters) may be combined with other polymers e.g. EUDRAGIT RL-30 D.

Other polymers may be used to provide a coating in particular enteric, or pH-dependent, polymers. Such polymers can include phthalate, butyrate, succinate, and/or mellitate groups. Such polymers include, but are not limited to, cellulose acetate phthalate, cellulose acetate succinate, cellulose hydrogen phthalate, cellulose acetate trimellitate, hydroxypropyl-methylcellulose phthalate, hydroxypropylmethylcellulose acetate succinate, starch acetate phthalate, amylose acetate phthalate, polyvinyl acetate phthalate, and polyvinyl butyrate phthalate.

### pH Independent Polymer Delayed Release Coatings

In a particular embodiment the second coating, where present, is or comprises a polymeric coating which is pH-independent in its dissolution profile and/or in its ability to release the active ingredient incorporated in the compositions of the invention. A pH-independent polymer delayed release coating comprises a delayed release polymer, optionally a plurality of delayed release polymers, and one or more other optional components. The other components may serve to modulate the properties of the composition. Examples have already been given (e.g., Eudragit RS and RL).

Another example of a pH-independent polymeric coating is a coating that comprises or is ethylcellulose; a pH-independent polymeric coating may have a delayed release polymer that is ethylcellulose, therefore. It will be understood that an ethylcellulose formulation for use in coating a dosage form may comprise, in addition to ethylcellulose and—in the case of a liquid formulation—a liquid vehicle, one or more other components. The other components may serve to modulate the properties of the composition, e.g. stability or the physical properties of the coating such as the flexibility of the film coating. The ethylcellulose may be the sole delayed release polymer in such a composition. The ethylcellulose may be in an amount of at least 50%, at least 60%, at least 70%, at least 80%, at least 90% or at least 95% by weight of the dry weight of a coating composition for use in coating a dosage form. Accordingly, an ethylcellulose coating may include other components in addition to the ethylcellulose. The ethylcellulose may be in an amount of at least 50%, at least 60%, at least 70%, at least 80%, at least 90% or at least 95% by weight of the ethylcellulose coating. Consequently, ethylcellulose may be in an amount of at least 50%, at least 60%, at least 70%, at least 80%, at least 90% or at least 95% by weight of the dry weight of the second coating. Suitably the ethyl cellulose coating further comprises a plasticizer as described below to improve the flexibility of the film and to improve the film-forming properties of the coating composition during application of the coating.

A particular ethylcellulose coating composition which may be applied to the composition, optionally to the core (i.e. in the absence of a first coating) or to the first coating is a dispersion of ethylcellulose in a sub-micron to micron particle size range, e.g. from about 0.1 to 10 μm in size, homogeneously suspended in water with the aid of an emulsification agent, e.g. ammonium oleate. The ethylcellulose dispersion may optionally and preferably contain a plasticizer. Suitably plasticisers include for example dibutyl sebacate (DBS), diethylphthalate, triethyl citrate, tributyl citrate, triacetin, or medium chain triglycerides. The amount of plasticizer present in the coating composition will vary depending upon the desired properties of the coating. Typically the plasticizer comprises from 1 to 50%, for example about 8 to about 50% of the combined weight of the plasticizer and ethyl cellulose. Such ethylcellulose dispersions may, for example, be manufactured according to U.S. Pat. No. 4,502,888, which is incorporated herein by reference. One such ethylcellulose dispersion suitable for use in the present invention and available commercially is marketed under the trademark Surelease®, by Colorcon of West Point, Pa. USA. In this marketed product, the ethylcellulose particles are, e.g., blended with oleic acid and a plasticizer, then optionally extruded and melted. The molten plasticized ethylcellulose is then directly emulsified, for example in ammoniated water optionally in a high shear mixing device, e.g. under pressure. Ammonium oleate can be formed in situ, for instance to stabilize and form the dispersion of plasticized ethylcellulose particles. Additional purified water can then be added to achieve the final solids content. See also U.S. Pat. No. 4,123,403, which is incorporated herein by reference.

The trademark “Surelease®” is used hereinafter to refer to ethylcellulose coating materials, for example a dispersion of ethylcellulose in a sub-micron to micron particle size range, e.g. from about 0.1 to 10 μm in size, homogeneously suspended in water with the aid of an emulsification agent, e.g. ammonium oleate. In particular, the trademark “Surelease®” is used herein to refer to the product marketed by Colorcon under the Surelease® trademark.

Surelease® dispersion is an example of a combination of film-forming polymer, plasticizer and stabilizers which may be used as a second coating to adjust rates of active principle release with reproducible profiles that are relatively insensitive to pH. The principal means of drug release is by diffusion through the Surelease® dispersion membrane and is directly controlled by film thickness. Use of Surelease® is particularly preferred and it is possible to increase or decrease the quantity of Surelease® applied as coating in order to modify the dissolution of the coated composition. Unless otherwise stipulated, use of the term “Surelease” may apply to Surelease E-7-19020, E-7-19030, E-7-19040 or E-7-19050. An ethylcellulose coating formulation, for example Surelease E-7-19020, may comprise ethylcellulose blended with oleic acid and dibutyl sebacate, then extruded and melted. The molten plasticized ethylcellulose is then directly emulsified in ammoniated water in a high shear mixing device under pressure. Ammonium oleate is formed in situ to stabilize and form the dispersion of plasticized ethylcellulose particles. Additional purified water is then added to achieve the final solids content. An ethylcellulose coating formulation, for example Surelease E-7-19030, may additionally comprise colloidal anhydrous silica dispersed into the material. An ethylcellulose coating formulation, for example Surelease E-7-19040, may comprise medium chain triglycerides instead of dibutyl sebacate, in particular in a formulation comprising colloidal anhydrous silica and oleic acid. An ethylcellulose coating formulation, for example Surelease E-7-19050, may derive from blending ethylcellulose with oleic acid before melting and extrusion. The molten plasticized ethylcellulose is then directly emulsified in ammoniated water in a high shear mixing device under pressure. Ammonium oleate is formed in situ to stabilize and form the dispersion of plasticized ethylcellulose particles. However, formulations that comprise medium chain triglycerides, colloidal anhydrous silica and oleic acid are preferred. Surelease E-7-19040 is particularly preferred.

The invention also contemplates using combinations of ethylcellulose, e.g. a Surelease formulation, with other coating components, for example sodium alginate, e.g. sodium alginate available under the trade name Nutrateric™.

In addition to the EUDRAGIT™ and Surelease® polymers discussed above, where compatible, any combination of coating polymers disclosed herein may be blended to provide additional delayed-release profiles.

The delayed release coating can further comprise at least one soluble excipient to increase the permeability of the polymeric material. These soluble excipients can also be referred to or as pore formers. Suitably, the at least one soluble excipient or pore former is selected from among a soluble polymer, a surfactant, an alkali metal salt, an organic acid, a sugar, a polysaccharide, and a sugar alcohol. Such soluble excipients include, but are not limited to, polyvinyl pyrrolidone, polyvinyl alcohol (PVA), polyethylene glycol, a water-soluble hydroxypropyl methyl cellulose, sodium chloride, surfactants such as, for example, sodium lauryl sulfate and polysorbates, organic acids such as, for example, acetic acid, adipic acid, citric acid, fumaric acid, glutaric acid, malic acid, succinic acid, and tartaric acid, sugars such as, for example, dextrose, fructose, glucose, lactose, and sucrose, sugar alcohols such as, for example, lactitol, maltitol, mannitol, sorbitol, and xylitol, xanthan gum, dextrins, and maltodextrins; and a polysaccharide susceptible of degradation by a bacterial enzyme normally found in the colon, for example polysaccharides include chondroitin sulphate, pectin, dextran, guar gum and amylase, chitosan etc. and derivatives of any of the foregoing. In some embodiments, polyvinyl pyrrolidone, mannitol, and/or polyethylene glycol can be used as soluble excipients. The at least one soluble excipient can be used in an amount ranging from about 0.1% to about 15% by weight, based on the total dry weight of the polymer coating, for example from about 0.5% to about 10%, about 0.5% to about 5%, about 1% to about 3%, suitably about 2% based on the total dry weight of the polymer coating. The delayed release coating may be free from HPMC.

The modifications in the rates of release, such as to create a delay or extension in release, can be achieved in any number of ways. Mechanisms can be dependent or independent of local pH in the intestine, and can also rely on local enzymatic activity to achieve the desired effect. Examples of modified-release compositions are known in the art and are described, for example, in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,566 all of which are incorporated herein by reference in their entirety.

The addition to Surelease™ or other pH-independent polymer substance of a second polymer (e.g. a polysaccharide, especially a heteropolysaccharide) which is susceptible to degradation by colonic bacterial enzymes (and optionally or alternatively by pancreatic or other relevant enzymes), helps provide targeted release of the active ingredient to a site or sites within the GI tract where the second polymer is degraded. By varying the amount of second polymer added present in the coating the dissolution profile may be optimized to provide the required release of NFAT inhibitor (e.g. cyclosporin) from the composition.

In a particular embodiments the delayed release coating provides for release of the active agent in at least the colon. Accordingly in one embodiment the coating comprises a ethylcellulose (preferably a described above, and particularly formulated with an emulsification agent such as, for example, ammonium oleate and/or a plasticizer such as, for example, dibutyl sebacate or medium chain triglycerides). In addition the coating may comprise a combination of ethylcellulose (preferably a described above, and particularly formulated with an emulsification agent such as, for example, ammonium oleate and/or a plasticizer such as, for example, dibutyl sebacate or medium chain triglycerides) and a polysaccharide susceptible of degradation by a bacterial enzyme normally found in the colon. Such polysaccharides include chondroitin sulphate, pectin, dextran, guar gum and amylase, chitosan etc. and derivatives of any of the foregoing. Chitosan may be used in connection with obtaining a colon-specific release profile; additionally or alternatively, pectin may be so used.

The use of polysaccharides by themselves for delayed release coating purposes has been tried with limited success. Most of the non-starch polysaccharides suffer from the drawback of lacking good film forming properties. Also, they tend to swell in the GI tract and become porous, resulting in the early release of the drug. Even amorphous amylose, which is resistant to degradation by pancreatic alpha amylase but capable of degradation by colonic bacterial enzymes, has the disadvantage of swelling in aqueous media although this can be controlled by incorporating insoluble polymer, for example ethyl cellulose and/or acrylate, into the amylose film. Amylose however is not water-soluble and although water-insoluble polysaccharides are not excluded, use of a water-soluble polysaccharide (WSP) susceptible to bacterial enzymatic degradation brings particularly advantageous results when used as a coating in accordance with this embodiment of the present invention. A particularly preferred polysaccharide in this embodiment of the present invention is pectin. Various kinds of pectin may be used including pectin of different grades available i.e. with differing degrees of methylation (DM), i.e. percentage of carbonyl groups esterified with methanol, for example pectins with a DM of more than 50%, known as High Methoxy (HM) Pectins or Low Methoxy (LM) pectins, or a pectin combination comprising an HM pectin and an LM pectin. It is also possible in this embodiment to use pectins having various degrees of acetylation (Dac). Taken together, the DM and Dac or the degree of substitution is known as Degree of Esterification (DE). Pectins of various DE's may be used according to the invention. As an alternative to pectin, sodium alginate may be used as a polysaccharide according to an embodiment of the invention. However, other embodiments may conveniently include amylose and/or starch which contains amylose. Various grades of starch, containing different percentages of amylose may be used including for example Hylon V (National Starch Food Innovation) which has an amylose percentage of 56% or Hylon VII which has an amylose percentage of 70%. The remaining percentage is amylopectin. The polysaccharides pectin, amylose and sodium alginate are particularly preferred for achieving colon delivery of the active ingredient.

It has been found that water-soluble polysaccharide, suitably pectin, can act as a former of pores in the coating otherwise provided by ethylcellulose (preferably Surelease). By “pores” is not meant shaft-like holes from the surface to the core of the composition, rather areas of weakness or absence of coating occurring stochastically on and within the coating of the invention.

Pore formers have been described before in connection with Surelease (see e.g. US 2005/0220878).

According to a particular embodiment of the invention the delayed release coating comprises ethylcellulose, e.g. Surelease™, and a water-soluble polysaccharide (WSP) wherein the proportion of ethylcellulose (in particular Surelease™) to WSP is ideally in the range 90:10 to 99:1, preferably, 95:5 to 99:1, more preferably 97:3 to 99:1, for example about 98:2 based upon the dry weight of the coating. Suitably in this embodiment the weight gain of the composition due to application of the coating comprising ethylcellulose, e.g. Surelease™, and the WSP is in the range of from 1 to 30% (for example from: 3% to 25%; 5% to 15%; 8% to 14%; 10% to 12%; 12% to 18%; or 16% to 18%, suitably the weight gain is about 11%, about 11.5%, or about 17%). It is particularly preferred that the WSP in this embodiment is pectin. Particularly favoured weight gains using coatings comprising ethylcellulose, e.g. Surelease™, are those in the range 5-12% or in the range 8-12%.

Accordingly in an embodiment the second coating comprises ethyl cellulose and a water soluble polysaccharide (particularly pectin) wherein the water-soluble polysaccharide (WSP) is present in an amount of 0% to 10%, optionally 0% or 0.1% to about 10% by weight, based on the dry weight of the second coating. Suitably the WSP is present in an amount of from about 0.5% to about 10%, for example about 0.5% to about 5%, about 1% to about 3%, suitably about 2% based on the total dry weight of the second coating. In this embodiment the WSP is preferably pectin. In this embodiment the second composition suitably further comprises a plasticizer. Suitable plasticizers include these described above in relation to Surelease™. Suitably the weight gain of the composition due to application of the second coating in this embodiment is in the range of from 1 to 30% (for example from: 3% to 25%; 5% to 15%; 8% to 14%; 10% to 12%; 12% to 18%; or 16% to 18%, suitably the weight gain is about 11%, about 11.5%, or about 17%).

In an embodiment the delayed release polymer is not a water-soluble cellulose ether. Where the second coating comprises or is a delayed release polymer the delayed release polymer may not be the same as the water-soluble cellulose ether of the first coating. Accordingly the second coating may not be the same as the first coating.

### First Coating

The invention provides pharmaceutical compositions that may have a first coating which is or comprises a water-soluble cellulose ether. The invention provides pharmaceutical compositions that have a first polymer coating, wherein the polymer is or comprises a water-soluble cellulose ether. The water-soluble cellulose ether may be, for example selected from methyl cellulose, hydroxyethyl cellulose, hydroxylpropyl cellulose and hydroxypropylmethyl cellulose.

Suitably the material of the first coating (i.e. the sub-coating) is different to the second coating on the composition. For example, where the first coating is or comprises a water-soluble ester of a cellulose ether, the major component(s) (e.g. more than 50%) of the second coating is or comprises a different polymer to that of the first coating. Accordingly, the first and second coatings suitably provide two layers of material as part of the composition. It is to be understood that when the second coating comprises a mixture of components, minor components of the outer second coating may the same as the material of the first coating. By way of example, when the first coating is or comprises HPMC and the second coating comprises ethyl cellulose, the ethyl cellulose may optionally further comprise a minor amount (e.g. less than 50%, 40%, 30% or 20%) of the first coating material, HPMC in this example. In such embodiments the sub-coat and the second coating are considered to be different.

The water-soluble cellulose ether may be a water-soluble cellulose ether selected from an alkyl cellulose, for example methyl cellulose, ethyl methyl cellulose; a hydroxyalkyl cellulose, for example hydroxyethyl cellulose (available as Cellosize™ and Natrosol™), hydroxypropyl cellulose (available as Klucel™) or hydroxymethyl cellulose; a hydroxyalkyl alkyl cellulose, for example hydroxyethyl methyl cellulose (NEMC), hydroxypropyl methyl cellulose (available as Methocel™ Pharmacoat™, Benecel™) or ethyl hydroxyethyl cellulose (EHEC); and a carboxyalkyl cellulose, for example carboxymethyl cellulose (CMC). Suitably the water-soluble cellulose ether may, for example be selected from methyl cellulose, hydroxyethyl cellulose, hydroxylpropyl cellulose and hydroxypopylmethyl cellulose.

The water-soluble cellulose ether may be a low viscosity polymer which is suitable for application as a film or coating to the composition. The viscosity of the polymer may be from about 2 to about 60 mPa·s, for example a viscosity of: about 2 to about 20 mPa·s; about to 2 to about 8 mPa·s; more suitably a viscosity of about 4 to about 10 mPa·s, for example about 4 to about 6 mPa·s. Alternatively, the viscosity of the polymer may fall outside any or all of the just-mentioned ranges, for example be above 20 mPa·s. Alternatively, the viscosity of the polymer may fall outside any or all of the just-mentioned ranges, for example be above 20 mPa·s. The viscosity of the polymer may be determined by measuring the viscosity of a 2% solution of the polymer in water at 20° C. using a Ubbelode viscometer using ASTM standard methods (D1347 and D2363).

The water soluble cellulose ether may be a water-soluble hydroxypropylmethyl cellulose (HPMC or hypromellose). HPMC is prepared by modifying cellulose to substitute hydroxy groups with methoxy and hydroxypropyl groups. Each anhydroglucose unit in the cellulose chain has three hydroxyl groups. The amount of substituent groups on the anhydroglucose units may be expressed as the degree of substitution. If all three hydroxyl groups on each unit are substituted, the degree of substitution is 3. The number of substituent groups on the ring determines the properties of the HPMC. The degree of substitution may also be expressed as the weight % of the methoxy and hydroxypropyl groups present. Suitably the HPMC has from about 19 to about 30% methoxy substitution and from about 7 to about 12% hydroxypropyl substitution. Particularly the HPMC has 25 to 30% methoxy substitution and 7 to 12% hydroxypropyl substitution. Suitably the HPMC is a low viscosity HPMC which is suitable for application as a film or coating to the composition. The viscosity of the HPMC is suitably from about 2 to 60 mPa·s, for example about 2 to about 20 mPa·s, more suitably a viscosity of about 4 to about 10 mPa·s. The viscosity of the HPMC is determined by measuring the viscosity of a 2% solution of the HPMC in water at 20° C. using a Ubbelode viscometer using ASTM standard methods (D1347 and D2363). Such HPMC is available as for example Methocel™, for example Methocel™ E, including Methocel™ E5.

When the first coating is or comprises a water-soluble derivative of a cellulose ether, the derivative may, for example be a water-soluble ester of a cellulose ether. Water-soluble esters of cellulose ethers are well known and may comprise esters of a cellulose ether, formed with one or more suitable acylating agent(s). Acylation agents may be, for example suitable acids or acid anhydrides or acyl halides. Accordingly the ester of a cellulose ether may contain a single ester moiety or two or more ester moieties to give a mixed ester. Examples of water-soluble esters of cellulose ethers may be water-soluble phthalate, acetate, succinate, propionate or butyrate esters of a cellulose ether (for example HPMC). Suitably the water-soluble ester of a cellulose ether is a water-soluble phthalate, acetate-succinate, propionate, acetate-propionate or acetate-butyrate ester of a cellulose ether (for example HPMC).

In one embodiment the water-soluble ester of a cellulose ether may be or comprise a water-soluble ester of any of the water-soluble cellulose ethers described above in relation to the sub-coating.

Particular water-soluble esters of cellulose ethers are water-soluble esters of HPMC. Esters of HPMC which are soluble in water at a pH greater than 5.5 may be or comprise hydroxypropyl methylcellulose phthalate (HPMCP), or hydroxypropyl methylcellulose acetate succinate (HPMCAS) in which the presence of ionisable carboxyl groups causes the polymer to solubilize at high pH (>5.5 for the LF grade and >6.8 for the HF grade). These polymers are commercially available from Shin-Etsu Chemical Co. Ltd.

The cellulose ether-containing coating may comprise or be hypromellose, e.g. it may be made of a mixture of hypromellose, titanium dioxide and polyethylene glycol; the coating may comprise at least 20 wt % hypromellose and optionally at least 50% or at least 75 wt % hypromellose, e.g. at least 80 wt % or at least 85 wt % or 90 wt % hypromellose. The coating material used to form the coating may therefore comprise a dry weight percentage of hypromellose mentioned in the preceding sentence.

If it is desired for the coating to use a mixture of hypromellose, titanium dioxide and polyethylene glycol, commercial products corresponding to such mixtures are available including Opadry White, a product commercialised by Colorcon. More generally, there may be mentioned various products commercialised under the trade name Opadry and Opadry II. Further non limiting examples include Opadry YS-1-7706-G white, Opadry Yellow 03692357, Opadry Blue 03690842). These formulations are available as dry film coating formulations that can be diluted in water shortly before use. Opadry and Opadry II formulations comprise a cellulosic film forming polymer (e.g., HPMC and/or HPC), and may contain polydextrose, maltodextrin, a plasticizer (e.g., triacetin, polyethylene glycol), polysorbate 80, a colorant (e.g., titanium dioxide, one or more dyes or lakes), and/or other suitable film-forming polymers (e.g., acrylate-methacrylate copolymers). Suitable OPADRY or OPADRY II formulations may comprise a plasticizer and one or more of maltodextrin, and polydextrose (including but not limited to a) triacetin and polydextrose or maltodextrin or lactose, or b) polyethylene glycol and polydextrose or maltodextrin). Particularly preferred commercial products are Opadry White (HPMC/HPC-based) and Opadry II White (PVA/PEG-based).

The cellulose ether-containing coating may also be applied as a simple solution comprising water and the polymer of the first coating. For example when the polymer is an HPMC, for example such as Methocel, the first coating may be applied to the core as an aqueous solution or dispersion of the HPMC. Optionally the coating solution may include other solvents such as an alcohol. Alternatively the coating may be applied as a solution or dispersion in a volatile organic solvent.

Suitably the first coating that contains a water soluble cellulose ether is present in an amount corresponding to a weight gain of the composition due to the coating of from 0.5% to 40% (for example from 0.5% to 30%; from 0.5% to 20%; from 1% to 25%; from 1% to 15%; from 1% to 6%; from 1% to 4%; from 4% to 6%; from 6% to 10%; from 9% to 15%; or from 12% to 15%) by weight based upon the weight of the composition prior to applying the coating. The first coating that contains a water soluble cellulose ether is present in an amount corresponding to a weight gain of the composition due to the coating of from 1% to 10%; from 4% to 10%; from 4% to 8%; and from 5% to 8% by weight based upon the weight of the core or the composition prior to applying the coating.

In another embodiment the first coating that contains a water-soluble cellulose ether is present in an amount corresponding to a weight gain due to the first coating in a range selected from 9 to 30%, suitably 9% to 20%, or particularly 10% to 15% by weight based upon the weight of the composition prior to applying the coating.

Suitably the first coating that contains a water soluble cellulose ether provides a coating thickness on the composition of at least 5 μm, suitably from about 5 μm to about 1 mm, for example from about 10 μm to about 1 mm, from about 10 μm to about 500 μm, from about 50 μm to about 1 mm, or about from about 50 μm to about 500 μm. The thickness may therefore be from about 100 μm to about 1 mm, e.g. 100 μm to about 750 μm or about 100 μm to about 500 μm. The thickness may be from about 250 μm to about 1 mm, e.g. about 250 μm to about 750 μm or 250 μm to about 500 μm. The thickness may be from about 500 μm to about 1 mm, e.g. about 750 μm to about 1 mm or about 500 μm to about 750 μm. The thickness may therefore be from about 10 μm to about 100 μm, e.g. from about 10 μm to about 50 μm or about 50 μm to about 100 μm.

When the first coating comprises a water-soluble cellulose ether the cellulose ether(s) suitably forms at least 40%, 50%, 60%, 70%, 80%, 85% or 90% by weight of the dry weight of the first coating. Alternatively the water-soluble cellulose ether is the first coating.

It is preferred to dry the composition of the invention before the first coating that contains a water-soluble cellulose ether is applied, as is described in more detail below in relation to the coating process.

It has been found that applying to a core comprising a pharmaceutically active ingredient a sub-coating, referred to elsewhere in the application as the subcoat (hence the subcoat and the first coating are equivalent), that contains a water soluble cellulose ether prior to applying a delayed release coating provides unexpected advantages. The presence of such a sub-coating has been found to enhance the dissolution properties of the delayed release compositions according to the invention. In particular the presence of such a sub-coating has been found to increase the rate of release of the active ingredient from the composition and also to increase the amount of the active ingredient released in a set time period compared to compositions prepared without using such a sub-coating. These findings are unexpected, because it would have been expected that the presence of a sub-coating in addition to a delayed release outer coating would act to delay or inhibit release of drug from the composition and, at a given time, for there to be less drug released, because there is a thicker coating present. However, contrary to these expectations both the extent and rate of release of active ingredient are increased compared to compositions without such a sub-coating. Accordingly, delayed release compositions according to the invention which comprise a sub-coat that comprises or is a water-soluble cellulose ether and a delayed release coating outside the sub-coat, provide a unique dissolution profile. The presence of such a sub-coating has also been found to reduce batch-to-batch variability, particularly when the core is in the form of a minibead. A sub-coating that comprises or is a water-soluble cellulose ether may therefore also reduce intra- and inter-patient variability as a result of a more consistent dissolution profile. The unique properties of sub-coated compositions according to the invention (particularly the dissolution profile) are expected to contribute to favourable pharmacokinetic properties of the compositions according to the invention. The coating containing the water-soluble cellulose ether of the present invention may be useful in reducing the variability between release profiles of different batches of minibeads.

Accordingly in an embodiment there is provided a composition comprising an NFAT inhibitor, a hydrogel forming polymer matrix, a surfactant and an oil phase being dispersed in the hydrogel forming polymer matrix, the composition further comprising a first coating; and wherein

the first coating is or comprises a water-soluble cellulose ether.

### Second Coating

The composition may have a second coating in addition to the first coating, wherein the second coating comprises or is a delayed release polymer. The composition may have a second coating as defined herein but the first coating may be absent. Similarly, the composition may have a first coating and the second coating may be absent.

The second coating may be present in a weight gain of from 4% to 25%, optionally from: 4% to 15%, 4% to 12%, 15% to 25%, 8% to 13%, 2% to 20%, 5% to 15%, 8% to 15%, 8% to 12%, 2% to 8%, 3% to 7%, or 4% to 6%.

In an embodiment there is provided a composition comprising an NFAT inhibitor, a hydrogel forming polymer matrix, a surfactant and an oil phase being dispersed in the hydrogel forming polymer matrix, the composition further comprising a first coating and a second coating outside the first coating; and wherein

the first coating is or comprises a water-soluble cellulose ether; and

the second coating is or comprises a delayed release coating, e.g. is or comprises a delayed release polymer.

In an embodiment there is provided a composition comprising cyclosporin, a hydrogel forming polymer matrix, a surfactant and an oil phase being dispersed in the hydrogel forming polymer matrix, the composition further comprising a second coating in the absence of a first coating; and wherein

the second coating is or comprises a delayed release coating, e.g. is or comprises a delayed release polymer.

An aspect of the invention resides in a multiple minibead composition comprising at least two populations of active ingredient-containing minibeads, wherein members of at least one minibead population are minibeads as described herein (i.e. compositions of the invention in minibead format). It will be understood that the two populations are different. Such a plural minibead population composition may comprise or consist of two or more of the following three populations:
a population having a coating that is or comprises a water-soluble cellulose ether but having no outer coating, e.g. as described herein; and/or
a population having a first coating that is or comprises a water-soluble cellulose ether and a second coating that is or comprises a delayed release coating, for example as described herein e.g. a coating that is or comprises a delayed release polymer; and/or
a population having a second coating that is or comprises a second coating that is or comprises a delayed release coating, for example as described herein e.g. a coating that is or comprises a delayed release polymer in the absence of a first coating.

The respective minibeads of each population of a plural minibead composition may contain an NFAT inhibitor (e.g. cyclosporin) and the minibeads of some or all of the other populations, or one population may contain an NFAT inhibitor (e.g. cyclosporin) and another population may contain a different active ingredient(s) thereto, e.g. a different combination.

A multiple population composition may be for use in delivering the NFAT inhibitor to different regions of the gastrointestinal tract.

The minibeads of a multiple population composition may by way of example be contained in a gel capsule or a sachet.

The second coating is outside the first coating and may be any of the delayed release coatings described herein. In particular, the second coating is or comprises a pH independent polymer modified release coating described above. For example the second coating may be or comprise an enteric coating or a pH independent coating. The second coating may comprise a mixture of polymers including a polymer degradable by bacterial or other enzymes. In a particular embodiment the second coating comprises ethyl cellulose and optionally a water-soluble polysaccharide, in particular one susceptible to degradation by colonic bacteria, suitably pectin. Accordingly the second coating may comprise the Surelease-pectin mixture described above.

It is not a requirement that both the first and second coatings are present in the composition at the same time. For example, the composition may comprise second coating (outer coating) in the absence of a first coating. Conversely, the composition may comprise a first coating in the absence of a second coating.

The first and second coating may independently be aqueous-based coatings or may be solvent-based coatings. By this it is meant that the first and/or second coating may be formulated prior to being applied to the core or composition and/or applied to the core or composition as an aqueous-based composition or as a solvent-based (non-aqueous solvent-based) composition. The aqueous-based or solvent-based coating compositions may be a suspension or a solution of the coating material in water or in a solvent.

In an embodiment the composition comprises a core and an outer coating (also referred to as a second coating herein), the core comprising an NFAT inhibitor (e.g. cyclosporin), a hydrogel forming polymer matrix, a surfactant and an oil phase being dispersed in the hydrogel forming polymer matrix, wherein the surfactant is a medium chain or long chain fatty acid mono- or di-glyceride or a combination thereof and does not comprise or is not a polyethyleneglycol ether or ester. The composition may optionally further comprise a sub-coat.

In one embodiment of the invention there is provided a composition comprising a core, a first coating and a second coating outside the first coating; and wherein:

the core comprises an NFAT inhibitor (e.g. cyclosporin), a hydrogel forming polymer matrix, a surfactant and an oil phase being dispersed in the hydrogel forming polymer matrix, wherein the surfactant is a medium chain or long chain fatty acid mono- or di-glyceride or a combination thereof and does not comprise or is not a polyethyleneglycol ether or ester;

the first coating is or comprises a water-soluble cellulose ether, particularly hydroxypropylmethyl cellulose;

the second coating is or comprises a modified release coating or delayed release coating, particularly a pH independent modified release coating;

the first coating is present in an amount corresponding to a weight gain due to the first coating in a range selected from: (i) 8% to 15%; (ii) from 8% to 12%, for example about 10%; or (iii) from 2.5% to 6%, for example about 5% by weight based upon the weight of the composition prior to applying the first coating; and wherein

the second coating is present in an amount corresponding to a weight gain of the composition due to the second coating selected from (a) from 4% to 20%; (b) from 7.5% to 20%; (C) from 10% to 12%, for example about 11% or about 11.5%; or (d) from 16% to 18%, for example about 17% by weight based upon the weight of the composition prior to applying the second coating.

The first and second coatings in the embodiment of the immediately preceding paragraph are suitably any of the first and second coatings described above or below. Accordingly it is intended that the coatings described in this section may be applied to any of the compositions described herein to provide a delayed release coating if required. The coatings are particularly useful to provide a modified release coating to the cores comprising a polymer matrix and pharmaceutically active ingredient described in this application.

### Outer Barrier or Protective Coating

The compositions described herein may comprise a protective coating outside the first and/or second coating, for example outside the second coating, the modified release coating. The protective coating may help to protect the modified release coating from damage resulting from, for example formulating the composition into a final dosage form, or during the handling, transport or storage of the composition. The protective coating is suitably applied to the outer surface of the composition. The protective coating may be applied directly to the second coating (the modified release coating) such that the protective coating is in contact with the second coating (the modified release coating). The protective coating is suitably a water soluble coating which does not adversely affect the release of the cyclosporin A from the composition when in use. Suitably the protective coating is or comprises a water-soluble polymer. The protective coating may comprise a water-soluble cellulosic or PVA film-forming polymer. Suitably the protective coating may be or comprise Opadry (HPMC/HPC-based), Opadry II (PVA/PEG-based) or polyvinyl alcohol-polyethylene glycol graft copolymers (Kollicoat IR) as described herein. The protective coating may be present as a layer of from about 2 to about 50 μm. Suitably the protective coating is applied to give a weight-gain of from about 0.5 to about 10%, based upon the weight of the composition prior to applying the protective coating.

### Continuous Phase Polymer Matrix (Aqueous Phase)

This section of the specification refers to a polymer matrix and continuous phase both of which concern the hydrogel forming polymer matrix. Therefore, reference to a polymer matrix or continuous phase can be equated to the hydrogel forming polymer matrix. Furthermore, this section of the specification relating to the polymer matrix recites amounts of constituents in terms of percent by weight of the composition. In the context of this section of the specification, what is meant is percent by weight of the dry weight of the composition or core excluding coating(s).

The composition or the core may comprise a matrix or continuous phase and also a disperse phase or oil phase. Similarly the liquid composition of the invention comprises an aqueous phase comprising a hydrogel forming polymer. Suitably the continuous phase or matrix phase of the composition or core is or comprises a hydrogel-forming polymer. A hydrogel-forming polymer is a polymer capable of forming a hydrogel. A hydrogel may be described as a solid or semi-solid material, which exhibits no flow when at rest, comprising a network (matrix) of hydrophilic polymer chains that span the volume of an aqueous liquid medium. A hydrogel forming polymer matrix is a network of hydrogel forming polymer chains, thus a hydrogel forming polymer matrix is a hydrogel forming polymer that has been allowed or caused to form a matrix.

The composition or core may comprise a hydrogel-forming polymer matrix selected from the group consisting of: gelatin; agar; agarose; pectin; carrageenan; chitosan; alginate; starch; xanthan gum; gum Arabic; guar gum; locust bean gum; polyurethane; polyether polyurethane; cellulose; cellulose ester, cellulose acetate, cellulose triacetate; cross-bonded polyvinyl alcohol; polymers and copolymers of acrylic acid, hydroxyalkyl acrylates, hydroxyethyl acrylate, diethylene glycol monoacrylate, 2-hydroxypropylacrylate, 3-hydroxypropyl acrylate; polymers and copolymers of methacrylic acid, hydroxyethyl methacrylate, diethyleneglycol monomethacrylate, 2-hydroxypropyl methacrylate, 3-hydroxypropyl methacrylate, dipropylene glycol monomethylacrylate; vinylpyrrolidone; acrylamide polymers and copolymers, N-methylacrylamide, N-propylacrylamide; methacrylamide polymers and copolymers, N-isopropylmethacrylamide, N-2-hydroxyethylmethacrylamide; and vinyl pyrrolidone; and combinations thereof. In specific embodiments binary or tertiary etc combinations of any of the above substances are foreseen.

In a further embodiment the hydrogel forming polymer matrix is selected from the group consisting of gelatin, agar, a polyethylene glycol, starch, casein, chitosan, soya bean protein, safflower protein, alginates, gellan gum, carrageenan, xanthan gum, phthalated gelatin, succinated gelatin, cellulosephthalate-acetate, oleoresin, polyvinylacetate, polymerisates of acrylic or methacrylic esters and polyvinylacetate-phthalate and any derivative of any of the foregoing; or a mixture of one or more such hydrogel-forming polymers.

The hydrogel forming polymer matrix may also be referred to as a hydrocolloid i.e. a colloid system wherein the colloid particles are disperse in water and the quantity of water available allows for the formation of a gel. In embodiments it is preferred to use reversible hydrocolloids preferably thermo-reversible hydrocolloids (e.g. agar, agarose, gelatin etc) as opposed to irreversible (single-state) hydrocolloids. Thermo-reversible hydrocolloids can exist in a gel and sol state, and alternate between states with the addition or elimination of heat. Gelatin, agar and agarose are thermo-reversible, rehydratable colloids and are particularly preferred. Gelatin derivatives such as, for example, succinated or phthalated gelatins are also contemplated. Thermoreversible hydrocolloids which may be used according to the invention, whether individually or in combination, include those derived from natural sources such as, for example, carrageenan (extracted from seaweed), gelatin (extracted from bovine, porcine, fish or vegetal sources), agar (from seaweed), agarose (a polysaccharide obtained from agar) and pectin (extracted from citrus peel, apple and other fruits). A non-animal based hydrocolloid may be preferred for certain applications e.g. administration to vegetarians or to individuals not wishing to ingest animal products for religious or health reasons. In relation to the use of carrageenan, reference is made to US patent application 2006/0029660 A1 (Fonkwe et al), the entirety of which is incorporated herein by reference. The hydrogel-forming polymer may comprise or be a combination of gelatin with one or more other thermoreversible hydrocolloids, e.g. with one or more other of the thermoreversible hydrocolloids just listed. The hydrogel-forming polymer may comprise or be a combination of gelatin with agar; optionally, at least one further thermoreversible hydrocolloid may be included in the combination, for example one just listed.

Thermo-reversible colloids present a benefit over other hydrogel-forming polymers. Gelation or hardening of thermo-reversible colloids occurs by cooling the colloid, e.g. in a liquid cooling bath or by air flow. Gelation of other hydrogel-forming polymers, which is chemically driven, can lead to leakage of the composition contents into the gelation medium as the hardening process can take time to occur. Leakage of the content of the composition may lead to an inaccurate quantity of the active ingredient within the composition. Thermo-reversible colloids are also known as thermo-reversible gels, and it is therefore preferred that the hydrogel former be a thermo-reversible gelling agent.

Another term which may be applied to hydrogel formers which are advantageous is “thermotropic”: a thermotropic gelling agent (which the reader will infer is preferred as a hydrogel former used in the invention) is one caused to gel by a change in temperature and such gelling agents are able to gel more rapidly than those whose gelling is chemically induced, e.g. ionotropic gelling agents whose gelling is induced by ions, for example chitosan. In embodiments of the invention, therefore, the hydrogel former is a thermotropic gel-forming polymer or a combination of such polymers.

The manufacture of the composition to prepare a core may require that the hydrogel-forming polymer be present as a solution, which is preferably an aqueous solution. The hydrogel-forming polymer represents between 5% and 50%, preferably between 10% and 30%, still more preferably between 15% and 20% by weight of the aqueous phase during manufacture as described herein. In addition the hydrogel-forming polymer may comprise 8 to 35%, (for example 15-25%, preferably 17-18%) hydro-gel forming polymer; 65%-85% (preferably 77-82%) of water plus, optionally, from 1-5% (preferably 1.5 to 3%) sorbitol. When present surfactant (e.g. anionic surfactant) in the aqueous phase pre-mix may be present in an amount of 0.1 to 5% (preferably 0.5 to 4%) wherein all parts are by weight of the aqueous phase.

In embodiments the composition comprises at least 25%, suitably at least 40% by weight based upon the dry weight of the composition of the hydrogel-forming polymer matrix. For example the hydrogel-forming polymer matrix is present from 25 to 70%, for example 40 to 70% suitably 45 to 60% of the composition, wherein the % is by weight based upon the dry weight of the composition.

In embodiments the hydrogel-forming polymer is a pharmaceutically acceptable polymer.

In certain embodiments the hydrogel-forming polymer is gelatin. In certain embodiments the hydrogel-forming polymer matrix is gelatin. In certain embodiments the hydrogel-forming polymer comprises gelatin. In certain embodiments the gelatin comprises at least 30%, for example 30 to 70% or 40 to 70% suitably 40 to 60% of the composition, wherein the % is by weight based upon the dry weight of the composition.

The hydrogel-forming polymer may optionally comprise a plasticiser for example sorbitol or glycerine, or a combination thereof. In particular one or more plasticisers may be combined with gelatin.

In embodiments in which the hydrogel-forming polymer comprises or is gelatin, reference is hereby made to “Bloom strength”, a measure of the strength of a gel or gelatin developed in 1925 by O. T. Bloom. The test determines the weight (in grams) needed by a probe (normally with a diameter of 0.5 inch) to deflect the surface of the gel 4 mm without breaking it. The result is expressed in Bloom (grades) and usually ranges between 30 and 300 Bloom. To perform the Bloom test on gelatin, a 6.67% gelatin solution is kept for 17-18 hours at 10° C. prior to being tested.

When the hydrogel-forming polymer comprises or is gelatin the bloom strength of the gelatin may be in the range of 125 Bloom to 300 Bloom, 200 Bloom to 300 Bloom and preferably 225 Bloom to 300 Bloom. It should be appreciated that higher bloom strength gelatin can be replaced by lower bloom strength gelatin at higher concentrations.

According to the invention, in embodiments in which the hydrogel forming polymer or hydrogel-forming polymer matrix comprises or is gelatin, the gelatin may be sourced by a variety of means. For example, it can be obtained by the partial hydrolysis of collagenous material, such as the skin, white connective tissues, or bones of animals. Type A gelatin is derived mainly from porcine skins by acid processing, and exhibits an isoelectric point between pH 7 and pH 9, while Type B gelatin is derived from alkaline processing of bones and animal (bovine) skins and exhibits an isoelectric point between pH 4.7 and pH 5.2. Type A gelatin is somewhat preferred. Gelatin for use in the invention may also be derived from the skin of cold water fish. Blends of Type A and Type B gelatins can be used in the invention to obtain a gelatin with the requisite viscosity and bloom strength characteristics for bead manufacture.

Lower temperature gelatin (or gelatin derivatives or mixtures of gelatins with melting point reducers) or other polymer matrices able to be solidified at lower temperatures (e.g. sodium alginate) may also be used. It is therefore believed that polymer which comprises or is low temperature gelatin is a preferred matrix polymer.

According to the invention, in embodiments in which the hydrogel forming polymer or hydrogel forming polymer matrix comprises or is gelatin, the starting gelatin material is preferably modified before manufacture to produce “soft gelatin” by the addition of a plasticizer or softener to the gelatin to adjust the hardness of the composition of the invention. The addition of plasticizer achieves enhanced softness and flexibility as may be desirable to optimise dissolution and/or further processing such as, for example, coating. Useful plasticizers of the present invention for combination with gelatin or another hydrogel-forming polymer include glycerine (1,2,3-propanetriol), D-sorbitol (D-glucitol), sorbitol BP (a non-crystallizing sorbitol solution) or an aqueous solution of D-sorbitol, sorbitans (e.g. Andidriborb 85/70), mannitol, maltitol, gum arabic, triethyl citrate, tri-n-butyl citrate, dibutylsebacate. Other or similar low molecular weight polyols are also contemplated for example ethylene glycol and propylene glycol. Polyethylene glycol and polypropylene glycol may also be used although these are less preferred. Glycerine and D-sorbitol may be obtained from the Sigma Chemical Company, St. Louis, Mo. USA or Roquette, France. Some active agents and excipients included for other functions may act as plasticisers.

Softeners or plasticisers, if utilized, can be ideally incorporated in a proportion rising to 30%, preferably up to 20% and more preferably up to 10% by dry weight of the composition of the invention, even more preferably between 3 and 8%, and most preferably between 4% and 6%.

Although not essential, the hydrogel-forming polymer matrix may also optionally contain a disintegrant where it is particularly desired to enhance the rate of disintegration of the composition of the invention. Examples of disintegrants which may be included are alginic acid, croscarmellose sodium, crospovidone, low-substituted hydroxypropyl cellulose and sodium starch glycolate.

A crystallisation inhibitor (e.g. approximately 1% by dry weight of the composition) may also be included in the composition of the invention. An example is hydroxy propyl/methyl cellulose (HPC or HPMC, hypromellose etc) which may play other roles such as, for example, emulsifier.

In another embodiment, the hydrogel-forming polymer matrix is chitosan which can exist in the form of biogels with or without additives as described e.g. in U.S. Pat. No. 4,659,700 (Johnson & Johnson); by Kumar Majeti N. V. Ravi in Reactive and Functional Polymers, 46, 1, 2000; and by Paul et al. in ST.P. Pharma Science, 10, 5, 2000 the entirety of all 3 of which is incorporated herein by reference. Chitosan derivatives e.g. thiolated entities are also contemplated.

The hydrogel-forming polymer matrix may be a non-hydrocolloid gum. Examples are the cross-linked salts of alginic acid. For example, aqueous solutions of sodium alginate gums extracted from the walls of brown algae have the well-known property of gelling when exposed to di- and trivalent cations. A typical divalent cation is calcium, often in the form of aqueous calcium chloride solution. It is preferred in this embodiment that the cross-linking or gelling have arisen through reaction with such a multivalent cation, particularly calcium.

The hydrogel-forming polymer matrix may have a low water content, therefore the composition may have a low water content. As described below, during manufacture of a core the disperse phase or oil phase, optionally comprising an NFAT inhibitor for example cyclosporin, is mixed with an aqueous solution of the hydrogel-forming polymer and the composition is gelled, for example to provide a composition or a core which are minibeads. Suitably the composition or cores are dried following formation to reduce the water content present therein.

In certain embodiments the composition does not comprise compounds containing a disulphide bond. In embodiments the hydrogel-forming polymer does not comprise compounds containing a disulphide bond.

The hydrogel-forming polymer matrix forming the continuous phase of the core (aqueous phase) may further comprise a surfactant. Surfactants which may be used in the composition are described in the section “surfactants” below.

Surfactant which may be present in the continuous phase, aqueous phase or the hydrogel forming polymer matrix of the composition or core include, for example a surfactant selected from the group consisting of: cationic; amphoteric (zwitterionic); anionic surfactants, for example perfluoro-octanoate (PFOA or PFO), perfluoro-octanesulfonate (PFOS), sodium dodecyl sulfate (SDS), ammonium lauryl sulfate, and other alkyl sulfate salts, sodium laureth sulfate, also known as sodium lauryl ether sulfate (SLES) and alkyl benzene sulphonate; and non-ionic surfactants for example perfluorocarbons, polyoxyethyleneglycol dodecyl ether (e.g. Brij such as, for example, Brij 35), Myrj (e.g. Myrj 49, 52 or 59), fatty alcohol ethoxylates, alkylphenol ethoxylate, fatty acid ethoxylates, fatty amide ethoxylates, alkyl glucosides, Tween 20 or 80 (also known as Polysorbate) (Brij, Myrj and Tween products are available commercially from Croda), poloxamers which are nonionic triblock copolymers composed of a central hydrophobic chain of polyoxypropylene (poly(propylene oxide)) flanked by two hydrophilic chains of polyoxyethylene (poly(ethylene oxide)), or a combination of the foregoing. In particular, the surfactant may be selected from, or comprise, anionic surfactants and combinations thereof, the anionic surfactants optionally being those mentioned in this paragraph. A particular class of surfactant comprises sulfate salts. A preferred anionic surfactant in the aqueous phase is SDS. Mixtures of anionic surfactants may be used. Mixtures of further surfactants are also contemplated, e.g. mixtures comprising perfluorocarbons.

In embodiments of the invention, the core comprises a hydrophilic surfactant which, without being bound by theory, is believed at least partially to partition the aqueous phase (polymer matrix).

Such surfactants intended for such inclusion in the aqueous phase of the core are preferably readily diffusing or diffusible surfactants to facilitate manufacturing and processing of the composition of the invention.

The surfactant may have an HLB of at least 10 and optionally of at least 15, e.g. at least 20, or at least 30 and optionally of 38-42, e.g. 40. Such surfactants can be of any particular type (ionic, non-ionic, zwitterionic) and may comprise as a proportion of dry weight of the composition from 0.1% to 6%, e.g. 0.1% to 5%. 0.1% to 4% or 0.1% to 3%, more preferably in a proportion of at least 1% and in particular between 1.0 and 4.5 or 5%, ideally within or just outside the 2-4% range, for example from 2 to 3% or approximately 2% or approximately 4%.

Unless otherwise stated or required, all percentages and ratios are by weight.

In one embodiment the anionic surfactant which may be present in the continuous phase, aqueous phase or the hydrogel forming polymer matrix of the composition or core may be an anionic surfactant selected from alkyl sulphates, carboxylates or phospholipids, or combinations thereof.

The physical form of the surfactant at the point of introduction into the aqueous phase during preparation of the composition or core plays a role in the ease of manufacture of the composition or core. As such, although liquid surfactants can be employed, it is preferred to utilize a surfactant which is in solid form (e.g. crystalline, granules or powder) at room temperature, particularly when the aqueous phase comprises gelatin.

### Disperse Phase

The polymer matrix or the continuous phase of the composition, or in embodiments where a core is present, the core described above (for example the hydrogel-forming polymer) may comprise a disperse phase which is or comprises the oil phase. Suitably the disperse phase, where present, may comprise the NFAT inhibitor (e.g. cyclosporin). In such embodiments the NFAT inhibitor (e.g. cyclosporin) is preferably soluble in the disperse phase, i.e. the disperse phase comprises a vehicle in which the active is dissolved. Embodiments wherein the cyclosporin is solubilised in the disperse phase are preferred, because such compositions release the cyclosporin in a solubilised form, which may enhance the therapeutic effect of the drug at the site of release, for example by enhancing absorption into the colonic mucosa.

In embodiments the NFAT inhibitor (e.g. cyclosporin) is or is comprised in the disperse phase. The disperse phase is or comprises the oil phase. Preferably, the disperse phase is the oil phase.

The disperse phase may comprise a water immiscible phase (also referred to herein as an oil phase). The water immiscible phase may be solid, semi-solid or liquid at ambient temperature (e.g. 25° C.), and therefore the oil phase may for example be waxy at ambient temperature. The oil phase may be or may comprise a liquid lipid and optionally a solvent miscible therewith. The NFAT inhibitor, for example cyclosporin, may be present in the oil phase. Suitably the NFAT inhibitor (e.g. cyclosporin) is soluble in the oil phase.

The disperse phase may comprise a combination of oils, for example liquid lipids. The oil phase may be or may comprise the liquid lipid. The liquid lipid may be a short-, medium- or long-chain triglyceride formulation, or a combination thereof. A medium chain triglyceride(s) (MCT) comprises one or more triglycerides of at least one fatty acid selected from C₆, C₇, C₈, C₉, C₁₀, C₁₁ and C₁₂ fatty acids. It will be understood that commercially available triglyceride, in particular MCT, formulations useful in the invention are mixtures derived from natural products and usually or always contain minor amounts of compounds which are not MCTs; the term “medium chain triglyceride formulation” is therefore to be interpreted to include such formulations. A short chain triglyceride(s) comprises one or more triglycerides of at least one short chain fatty acid selected from C₂-C₅ fatty acids. A long chain triglyceride(s) comprises one or more triglycerides of at least one long chain fatty acid having at least 13 carbon atoms.

The liquid lipid may comprise or be triglycerides and/or diglycerides. Such glycerides may be selected from medium chain triglycerides or short chain triglycerides or a combination thereof.

The liquid lipid may be a caprylic/capric triglyceride, i.e. a caprylic/capric triglyceride formulation (which it will be understood may contain minor amounts of compounds which are not caprylic/capric triglycerides). Accordingly, the oil phase may be triglycerides and/or diglycerides, optionally a caprylic/capric triglyceride, i.e. a caprylic/capric triglyceride formulation.

The disperse phase may optionally comprise a solvent. Accordingly the oil phase may comprise a solvent. Said solvent which is optionally included in an oil phase may be miscible with both the liquid lipid and with water. Examples of suitable solvents are 2-(2-ethoxyethoxy)ethanol available commercially under trade names Carbitol™ Carbitol cellosolve, Transcutol™, Dioxitol™ Poly-solv DE™, and Dowanal DE™; or the purer Transcutol™ HP (99.9). Transcutol P or HP, which are available commercially from Gattefosse, are preferred. Another possible co-solvent is poly(ethylene glycol). PEGs of molecular weight 190-210 (e.g. PEG 200) or 380-420 (e.g. PEG 400) are preferred in this embodiment. Suitable PEGs can be obtained commercially under the name “Carbowax” manufactured by Union Carbide Corporation although many alternative manufacturers or suppliers are possible.

The disperse phase may represent from 10-85% by dry weight of the core.

As discussed above the disperse phase may be an oil phase comprising any pharmaceutically suitable oil, e.g. a liquid lipid. The oil phase may be present as oil drops. In terms of dry weight of the core, the oil phase may comprise a proportion from 10% to 85%, e.g. 15% to 50%, for example 20% to 30% or from 35% to 45%. The term “oil” means any substance that is wholly or partially liquid at ambient temperature or close-to-ambient temperature e.g. between 10° C. and 40° C. or between 15° C. and 35° C., and which is hydrophobic but soluble in at least one organic solvent. Oils include vegetable oils (e.g. neem oil) and petrochemical oils.

The oil may be present in the composition in an amount of from about 2% to about 25%, from about 3% to about 20%, from about 3% to about 10% or from about 5% to about 10% by weight based upon the dry weight of the core.

Oils which may be included in the oil phase include poly-unsaturated fatty acids such as, for example, omega-3 oils for example eicosapentanoic acid (EPA), docosohexaenoic acid (DHA), alpha-linoleic acid (ALA), conjugated linoleic acid (CLA). Preferably ultrapure EPA, DHA or ALA or CLA are used e.g. purity up to or above 98%. Omega oils may be sourced e.g. from any appropriate plant e.g. sacha inchi. Such oils may be used singly e.g. EPA or DHA or ALA or CLA or in any combination. Combinations of such components including binary, tertiary etc combinations in any ratio are also contemplated e.g. a binary mixture of EPA and DHA in a ratio of 1:5 available commercially under the trade name Epax 6000. The oil part of the oil phase may comprise or be an oil mentioned in this paragraph.

Oils which may be included in the oil phase are particularly natural triglyceride-based oils which include olive oil, sesame oil, coconut oil, palm kernel oil, neem oil. The oil may be or may comprise saturated coconut and palm kernel oil-derived caprylic and capric fatty acids and glycerin e.g. as supplied under the trade name Miglyol™ a range of which are available and from which one or more components of the oil phase of the invention may be selected including Miglyol™ 810, 812 (caprylic/capric triglyceride); Miglyol™ 818: (caprylic/capric/linoleic triglyceride); Miglyol™ 829: (caprylic/capric/succinic triglyceride; Miglyol™ 840: (propylene glycol dicaprylate/dicaprate). Note that Miglyol™ 810/812 are MCT formulations which differ only in C₈/C₁₀-ratio and because of its low C₁₀-content, the viscosity and cloud point of Miglyol™ 810 are lower. The Miglyol™ range is available commercially from Sasol Industries. As noted above, oils which may be included in the oil phase need not necessarily be liquid or fully liquid at room temperature. Waxy-type oils are also possible: these are liquid at manufacturing temperatures but solid or semi-solid at normal ambient temperatures. The oil part of the oil phase may comprise or be an oil mentioned in this paragraph.

Alternative or additional oils which may be included in the oil phase according to the invention are other medium chain triglyceride formulations such as for example Labrafac™ Lipophile manufactured by Gattefosse in particular product number WL1349. Miglyol™ 810, 812 are also medium chain triglyceride formulations.

Accordingly the oil phase may be or comprise medium chain mono-di- or tri-glycerides.

The medium chain glyceride(s) (eg mono- di- or tri-glyceride(s)) mentioned herein are those which comprise one or more triglycerides of at least one fatty acid selected from fatty acids having 6, 7, 8, 9, 10, 11 or 12 carbon atoms, e.g. C₈-C₁₀ fatty acids.

Suitable oils which may comprise or be the oil phase or disperse phase with a low HLB (HLB less than 10) include medium chain triglycerides, caprylocaproyl macrogolglycerides and caprylic/capric triglyceride. In terms of commercial products, particularly preferred oils in the lower HLB range are Labrafac™ Lipophile (e.g. 1349 WL), Captex 355 and Miglyol 810.

It is to be understood that the oil phase or disperse phase in the embodiments above may further comprise one or more solvents, for example 2-(2-ethoxyethoxy)ethanol or low molecular weight PEG as mentioned above. The solvent may be present in the composition in an amount of form about 1% to 30%, for about 5% to about 30%, for about 10% to about 25%, or from about 12% to about 22% by weight based upon the dry weight of the uncoated composition or upon the dry weight of the core.

A particular oil phase comprises an oil (low HLB), the surfactant and a co-solvent. For example the following three commercial products: Transcutol P (as co-solvent), Myglyol 810 (as oil) and Capmul GMO-50 (surfactant). An oil phase may therefore comprise or consist of a combination of the following: 2-ethoxyethanol, an MCT and particularly a caprylic/capric triglyceride formulation, and glyceryl monooleate/dioleate. The oil phase may further comprise the NFAT inhibitor, for example cyclosporin.

Preferably, the NFAT inhibitor, for example cyclosporin, is soluble in the oil phase. As discussed below in relation to preparation of the composition, the NFAT inhibitor, for example cyclosporin, is suitably dissolved in the oil phase and the oil phase is mixed with an aqueous phase comprising the hydrogel-forming polymer.

The disperse phase (oil phase) may be or comprise a glyceride formulation, optionally wherein the disperse phase is or comprises a fatty acid monoglyceride, diglyceride or triglyceride or a combination thereof, or the disperse phase is or comprises a caprylic/capric triglyceride formulation.

The oil phase may also include one or more volatile or non-volatile solvents, which may be the same or different from the solvent or co-solvent previously mentioned. Such solvents may for example remain in the formulation of the invention following processing e.g. initial dissolution of the components present in the core, and have no particular function in the core formulation. Alternatively, such solvents if present may function to maintain the NFAT inhibitor, for example cyclosporin, in a dissolved state (in solution) within the oil phase or to facilitate dispersion, egress etc. In other embodiments, the solvent may have partly or fully evaporated during processing and therefore be present in only minor quantities if at all. In a related embodiment, the solvent, particularly when a solvent which is both oil and water-soluble is used, may be partly or completely present in the aqueous phase of the core. An example of such a solvent is ethanol. Another example is transcutol which is already mentioned as a co-solvent.

Accordingly, the composition may comprise a hydrogel-forming polymer matrix which forms a continuous phase and a disperse phase comprising cyclosporin, a low HLB medium or long chain mono- or di-ester surfactant, a low HLB oil, and optionally a co-solvent. Optionally, the medium or long-chain mono- or di-ester surfactant is a medium- or long-chain mono- or di-glyceride surfactant.

### Surfactant

The oil phase may further comprise the surfactant as described above and elsewhere herein. The presence of the surfactant in the oil phase may also provide a stabilising effect on the liquid composition when the oil phase is dispersed in the aqueous phase. In addition the presence of the surfactant in the oil phase may inhibit crystallisation of the NFAT inhibitor, particularly where the inhibitor is cyclosporin from a solution of the NFAT inhibitor in the oil phase. The surfactant may also provide enhanced emulsification when the disperse phase is mixed with the aqueous phase during preparation of the liquid composition, composition or core (i.e. act as an emulsifier).

The surfactant may have an HLB value of up to 8, up to 6, or up to 5. Alternatively the surfactant may have an HLB value selected from: up to 7, 1-8, 1-7, 2-6, 1-5, 2-5, 1-4, 1-3, 1-2, 2-4, 3-4, 3-6, 5-8, 6-8 and 6-7. Preferably, the surfactant has an HLB value of up to 6, 2-6 or 3-6.

The NFAT inhibitor (e.g. cyclosporin) may be soluble in the surfactant, for example the cyclosporin A may have a solubility of more than 200 mg/g in the surfactant. Thus, the surfactant may have a cyclosporin solubility of more than 200 mg/g. The surfactant may have a cyclosporin solubility of from 200 mg/g to 500 mg/g, optionally from 250 mg/g to 500 mg/g.

The surfactant may have a NFAT inhibitor (e.g. cyclosporin) solubility of from 200 mg/g to 400 mg/g, from 225 mg/g to 375 mg/g, from 200 mg/g to 300 mg/g, from 300 mg/g to 400 mg/g, from 225 mg/g to 275 mg/g, from 350 mg/g to 400 mg/g. Preferably, the surfactant has a NFAT inhibitor (e.g. cyclosporin) solubility of from 200 mg/g to 400 mg/g or from 225 mg/g to 375 mg/g. The surfactant may have a NFAT inhibitor (e.g. cyclosporin) solubility of from 250 mg/g to 400 mg/g, from 250 mg/g to 375 mg/g, from 250 mg/g to 300 mg/g, from 300 mg/g to 400 mg/g, from 250 mg/g to 275 mg/g, from 350 mg/g to 400 mg/g. Preferably, the surfactant has a NFAT inhibitor (e.g. cyclosporin) solubility of from 250 mg/g to 400 mg/g or from 250 mg/g to 375 mg/g. The solubility of cyclosporin in a surfactant may be determined by techniques known to those skilled in the art, for example by following the protocol described in Development of a Self Micro-Emulsifying Tablet of Cyclosporine-A by the Liquisolid Compact Technique, Zhao et al (International Journal of Pharmaceutical Sciences and Research, 2011, Vol. 2(9), 2299-2308) which is incorporated herein by reference.

The surfactant may have an HLB of up to 6 and a NFAT inhibitor (e.g. cyclosporin) solubility of from 200 mg/g to 400 mg/g. The surfactant may have an HLB value of 2-6 (optionally 3-6) and a NFAT inhibitor (e.g. cyclosporin) solubility of from 200 mg/g to 400 mg/g. The surfactant may have an HLB value of 2-6 (optionally 3-6) and a NFAT inhibitor (e.g. cyclosporin) solubility of from 225 mg/g to 275 mg/g. The surfactant may have an HLB value of 2-6 (optionally 3-6) and a NFAT inhibitor (e.g. cyclosporin) solubility of from 250 mg/g to 300 mg/g.

The surfactant may have an HLB of up to 6 and a NFAT inhibitor (e.g. cyclosporin) solubility of from 250 mg/g to 400 mg/g. The surfactant may have an HLB value of 2-6 (optionally 3-6) and a NFAT inhibitor (e.g. cyclosporin) solubility of from 250 mg/g to 400 mg/g. The surfactant may have an HLB value of 2-6 (optionally 3-6) and a NFAT inhibitor (e.g. cyclosporin) solubility of from 250 mg/g to 375 mg/g. The surfactant may have an HLB value of 2-6 (optionally 3-6) and a NFAT inhibitor (e.g. cyclosporin) solubility of from 250 mg/g to 300 mg/g.

The presence of the surfactant may enhance the rate and or extent of release of the NFAT inhibitor, particularly cyclosporin from the composition following oral administration. The presence of the surfactant may act to maintain a high proportion of the NFAT inhibitor (e.g. cyclosporin) in a solubilised form after it has been released from the composition into an aqueous medium such as that found in the lower GI tract, particularly the colon.

In an embodiment the oil phase comprises an oil or liquid lipid and the surfactant is present in an amount greater than the oil or liquid lipid. Optionally, the surfactant may be present in an amount of more than 6 wt % of the dry weight of the composition. This refers to the uncoated composition or the core. The surfactant may comprise more than 12 wt % of the oil phase, for example in the liquid composition. The surfactant may be present in the composition in an amount of from about 5% to about 20%, from about 8% to about 20%, from about 8% to about 15%, or from about 10% to about 14% by weight based upon the dry weight of the core. It is to be understood that reference to the “dry weight of the core” means the weight of the components present in the uncoated core other than water.

The weight ratio of the surfactant:oil may be from about 5:1 to about 1:5, from about 3:1 to about 1:2, from about 3:1 to about 1:1 or from about 2.5:1 to 1.5:1. Suitably the weight ratio may be about 1:1, about 2:1, about 2.5:1, about 3:1, about 1:1.5 or about 1:2.

There is provided a composition comprising an NFAT inhibitor (optionally cyclosporin), a hydrogel forming polymer matrix, a surfactant and an oil phase being dispersed in the hydrogel forming polymer matrix, wherein the surfactant comprises or is a surfactant selected from: glyceryl caprylate/caprate (Capmul MCM), glyceryl monooleate/dioleate (Capmul GMO-50), glycerol monolinoleate (Maisine 35-1) and a combination thereof. The composition may be a solid composition. The composition may be in the form of a dried bead. The composition may be in the form of a dried colloid.

Optionally, the surfactant is or comprises glyceryl monooleate, glyceryl dioleate or a combination thereof. Capmul GMO-50 is an example of a commercially available surfactant that comprises a combination of glyceryl monooleate and glyceryl dioleate. Thus, the surfactant may be Capmul GMO-50. Where Capmul GMO-50 is mentioned in the specification it will be understood that it is referring to a mixture of glyceryl monooleate and glyceryl dioleate. Capmul GMO-50 may also refer to glyceryl monooleate alone.

Similarly, the skilled person would understand that a surfactant that is described as, for example glyceryl monooleate/dioleate, contemplates a combination of glyceryl monooleate and glyceryl dioleate. In other words a “/” in a surfactant name indicates that the surfactant is a mixture of two components.

The liquid lipid or oil of the oil phase or disperse phase is suitably not a surfactant. However, certain oils, particularly those derived from natural sources will comprise components which may have surface active properties. For example many triglyceride oils also comprise mono and diglyceride components and may therefore exhibit some surfactant like properties. Accordingly the oil suitably has an HLB value of 0-10, however suitably the oil has an HLB which is close to 0 for example an HLB of 0 to 3, optionally about 0, about 1 or about 2.

Surfactant in the oil phase may for example be or comprise a medium chain or long chain fatty acid mono- or di-glyceride or a combination thereof, wherein the surfactant does not comprise or is not a polyethyleneglycol ether or ester. Optionally the surfactant is a medium chain or long chain fatty acid mono-glyceride, di-glyceride or a combination thereof, optionally wherein the surfactant does not comprise or is not a polyethyleneglycol ether or ester. Two particular surfactants contemplated by the invention are glyceryl caprylate/caprate and glyceryl monooleate/dioleate. Commercial preparations may also be used as a surfactant e.g. those commercial preparations which contain minor components. Preferred examples are Capmul GMO-50 (glyceryl monooleate/dioleate) and Capmul MCM (glyceryl caprylate/caprate).

Within embodiments, the HLB of the oil may be in the range 0-10 (optionally 1-8, e.g. 1-6 and sometimes 1-5).

In another embodiment the oil phase comprises an oil with an HLB in the range 0-10 (preferably 1-5) and the has an HLB of up to 10 and optionally up to 7, 1-8, 1-7, 1-5, 2-5, 1-4, 1-3, 1-2, 2-4, 3-4, 5-8, 6-8 and 6-7.

In another embodiment the oil phase comprises an oil and the surfactant wherein the oil and the surfactant both have an HLB in the range 0-10. For example the oil has an HLB of 1-5, for example 1 to 4 or 1-2 and the surfactant has an HLB 2-8, for example 3-7, 2-6, or 3-4).

The composition comprises a surfactant, as described above. The surfactant may be present in the composition or the core, for example in the hydrogel-forming polymer matrix, or in the disperse phase or both. The surfactant may also be present in one or more of the coatings comprised in the composition or applied to the core.

The composition may comprise a further surfactant. Where the composition comprises a further surfactant this surfactant can be referred to as a second surfactant and the surfactant present in the composition of the invention can be referred to as a first surfactant. Accordingly, the first and second surfactant may be any surfactant detailed herein. In an embodiment the first surfactant is or comprises the medium chain or long chain fatty acid mono- or di-glyceride or a combination thereof, which does not comprise or is not a polyethyleneglycol ether or ester. In an alternative embodiment the first surfactant is an anionic surfactant, for example sodium lauryl sulphate. The further surfactant may be present in the composition or the core, for example in the hydrogel-forming polymer matrix, or in the disperse phase or both. The further surfactant may also be present in one or more of the coatings comprised in the composition or applied to the core. Suitable further surfactants can be anionic, cationic, zwitterionic, or non-ionic.

In the description and claims of this specification, the term “surfactant” is employed as a contraction for “surface active agent”. For the purposes of this description and claims, it is assumed that there are four major classifications of surfactants; therefore the further surfactant may be: anionic, cationic, non-ionic, and amphoteric (zwitterionic). The non-ionic surfactant remains whole, has no charge in aqueous solutions, and does not dissociate into positive and negative ions. Anionic surfactants are water-soluble, have a negative charge and dissociate into positive and negative ions when placed in water. The negative charge lowers the surface tension of water and acts as the surface-active agent. Cationic surfactants have a positive charge, and also dissociate into positive and negative ions when placed in water. In this case, the positive ions lower the surface tension of the water and act as the surfactant. The amphoteric (zwitterionic) surfactant assumes a positive charge in acidic solutions and performs as a cationic surfactant, or it assumes a negative charge in an alkaline solution and acts as an anionic surfactant.

The surfactant (the first surfactant and the second surfactant) may be selected from: anionic surfactants and combinations thereof; from non-ionic surfactants and combinations thereof; and from combination of an anionic surfactant (e.g. a single such surfactant or a plurality thereof) and a non-ionic surfactant (e.g. a single such surfactant or a plurality thereof). Preferably the surfactant is an anionic surfactant or a non-ionic surfactant. For example, the first surfactant may be non-ionic and the second surfactant may be anionic.

Furthermore, in an embodiment the composition comprises an NFAT inhibitor, a hydrogel forming polymer matrix, a first surfactant and an oil phase being dispersed in the hydrogel forming polymer matrix, the composition further comprising a second surfactant. Optionally, the first surfactant is or comprises non-ionic surfactant, for example a medium chain or long chain fatty acid mono- or di-glyceride or a combination thereof and does not comprise or is not a polyethyleneglycol ether or ester. Optionally, the second surfactant is an anionic surfactant.

Surfactants can be classified according to their hydrophilic-lipophilic balance (HLB) which is a measure of the degree to which the surfactant is hydrophilic or lipophilic, determined by calculating values for the different regions of the molecule, as described (originally for non-ionic surfactants) by Griffin in 1949 and 1954 and later by Davies. The methods apply a formula to the molecular weight of the whole molecule and of the hydrophilic and lipophilic portions to give an arbitrary (semi-empirical) scale up to 40 although the usual range is between 0 and 20. An HLB value of 0 corresponds to a completely hydrophobic molecule, and a value of 20 would correspond to a molecule made up completely of hydrophilic components. The HLB value can be used to predict the surfactant properties of a molecule:

| HLB Value | Expected properties |
|---|---|
| 0 to 3 | antifoaming agent |
| from 4 to 6 | W/O emulsifier |
| from 7 to 9 | wetting agent |
| from 8 to 18 | an O/W emulsifier |
| from 13 to 15 | typical of detergents |
| 10 to 18 | solubiliser or hydrotrope |

Although HLB numbers are assigned to surfactants other than the non-ionic, for which the system was invented, HLB numbers for anionic, cationic, non-ionic, and amphoteric (zwitterionic) surfactants can have less significance and often represent a relative or comparative number and not the result of a mathematical calculation. This is why it is possible to have surfactants above the “maximum” of 20. HLB numbers can however be useful to describe the HLB requirement of a desired application for a given emulsion system in order to achieve good performance.

The surfactant may be a non-ionic surfactant. The surfactant may be a polyoxyethylated surfactant. The surfactant has a hydrophilic head which may be a hydrophilic chain, for example a polyoxyethylene chain or a polyhydroxylated chain.

The surfactant of course has a hydrophobic part and in particular a hydrophobic chain. The hydrophobic chain may be a hydrocarbon chain, for example having at least 6 carbon atoms and optionally at least 10 carbon atoms, and particularly of at least 12 carbon atoms; some hydrocarbon chains have no more than 22 carbon atoms, for example C₁₀-C₂₀, C₁₂-C₂₀ or C₁₅-C₂₀ hydrocarbon chains. It may be an alkyl chain, e.g. having a number of carbon atoms just mentioned. It may be an alkenyl chain comprising one or more carbon-carbon double bonds, e.g. having a number of carbon atoms just mentioned. The surfactant may comprise a hydrocarbon chain, e.g. alkyl chain or alkenyl chain that is substituted provided that it maintains a hydrophobic characteristic. There may for example be one or two substituents, for example a single substituent, e.g. selected from halogen (e.g. F or Cl), hydroxy, thiol oxo, nitro, cyano; hydroxy or thiol substituents may be esterified by for example a fatty acid. One class of surfactants comprise a hydrocarbon monosubstituted by hydroxy; optionally, at least a portion of the hydroxy groups of an aliquot of surfactant, e.g. of the surfactant in a bead, may be esterified by a fatty acid or mono-hydroxy fatty acid as disclosed herein or etherified by a fatty alcohol for example having at least 6 carbon atoms and optionally at least 10 carbon atoms, and particularly of at least 12 carbon atoms; some hydrocarbon chains have no more than 22 carbon atoms, for example C₁₀-C₂₀, C₁₂-C₂₀ or C₁₅-C₂₀ fatty alcohols.

The hydrophobic chain may be part of an esterified fatty acid R¹—COOH or of an etherified or esterified fatty ether R¹—COH where R¹ is the hydrophobic chain, e.g. as mentioned in the preceding paragraph. The ester-forming or, as the case may be, ether-forming group will typically comprise a hydrophilic chain.

As mentioned, the surfactant may have a hydrophilic chain and may be a non-ionic surfactant, and may satisfy both requirements. The hydrophilic chain may be a poly(ethyleneglycol), also known as poly(oxyethylene) or macrogol. The hydrophilic chain may be of the formula —(O—CH₂—CH₂)ₙ—OR where n is 5 or 6 to 50 and R is H or alkyl, e.g. ethyl or methyl. The invention includes implementations in which n is from 6 to 40, e.g. from 6 to 35. In some embodiments, n is from 6 to 25 and optionally is from 8 to 25 or from 8 to 15. In other embodiments, n is from 8 to 50 or from 8 to 40, e.g. is from 10 to 50, 10 to 40 or 10 to 35. In a particular embodiment, n is 15. For all hydrophilic chains of the formula —(O—CH₂—CH₂)ₙ—OR, in one class of embodiments R is H.

The hydrophilic chain may be a polyhydroxylated chain (for example a C₅-C₂₀ e.g. C₅-C₁₀ chain), e.g. having a hydroxy group on the carbon atoms of the chain, for example a glucamide.

The surfactant may be a polyethoxylated castor oils (polyethylene glycol ethers) which can be prepared by reacting ethylene oxide with castor oil. Commercial preparations may be used as the surfactant e.g. those commercial preparations which contain minor components such as, for example, polyethyelene glycol esters of ricinoleic acid, polyethyelene glycols and polyethyelene glycol ethers of glycerol. The preferred example is Cremophor by BASF Corp. also known as Cremophor EL. Alternative or additional solubilizers include phospholipids such as, for example, phosphatidylcholine. In embodiments of the composition of the invention which comprise a phospholipid solubilizer, the phospholipid solubilizer may be incorporated either in the aqueous phase or in the oil phase or both. If at least one phospholipid solubilizer is incorporated in each phase, it may be the same phospholipid solubilizer in both phases or different in each.

### Non-Ionic Surfactants

The surfactant (first surfactant and/or second surfactant) may be or comprise at least one surfactant selected from the following non-ionic surfactants.

A medium chain or long chain fatty acid mono- or di-glyceride or a combination thereof that does not comprise or is not a polyethyleneglycol ether or ester selected from: glyceryl monocaprate, glyceryl dicaprate, glyceryl monocaprylate, glyceryl dicaprylate, glyceryl caprate, glyceryl monocaprylate/caprate, glyceryl caprylate/caprate glyceryl dicaprylate/caprate, glyceryl monooleate/dioleate, glyceryl monooleate, glyceryl dioleate, glyceryl monostearate, glyceryl distearate, glyceryl monopalmitostearate, glyceryl dipalmitostearate, glyceryl monobehenate, glyceryl dibehenate, glycerol monolinoleate, glyceryl dilinoleate, polyglyceryl dioleate, propylene glycol monoheptanoate, and a combination thereof.

PEG-fatty acid monoester surfactants, PEG-fatty acid diester surfactants, PEG-fatty acid monoester and diester surfactant mixtures, PEG glycerol fatty acid esters, transesterified products of oils and alcohols, lower alcohol fatty acid esters, polyglycerised fatty acids, propylene glycol fatty acid esters, mono and diglyceride surfactants, sterol and sterol derivative surfactants, PEG-sorbitan fatty acid esters, sorbitan fatty acid esters, polyethylene glycol alkyl ethers, sugar ester surfactants, polyethylene glycol alkyl phenol surfactants, POE-POP block copolymers, phospholipids.

A PEG-fatty acid mono ester surfactant for example PEG 4-100 monolaurate, PEG 4-100 monooleate, PEG 4-100 monostearate, PEG-laurate, PEG-oleate, PEG stearate, and PEG ricinoleate. A PEG-fatty acid diester surfactant for example PEG dilaurate; PEG dioleate, PEG distearate, PEG dipalmitate. A mixture of PEG-fatty acid mono- and diesters.

A PEG glycerol fatty acid ester for example PEG glyceryl laurate, PEG glyceryl stearate, PEG glyceryl oleate.

PEG-sorbitan fatty acid esters for example PEG sorbitan laurate, PEG sorbitan monolaurate, PEG sorbitan monopalmitate, PEG sorbitan monostearate, PEG sorbitan tristearate, PEG sorbitan tetrastearate, PEG sorbitan monooleate, PEG sorbitan oleate, PEG sorbitan trioleate, PEG sorbitan tetraoleate, PEG sorbitan monoisostearate, PEG sorbitol hexaoleate, PEG sorbitol hexastearate.

Propylene glycol fatty acid esters for example propylene glycol monocaprylate, propylene glycol monolaurate, propylene glycol oleate, propylene glycol myristate, propylene glycol monostearate, propylene glycol ricinoleate, propylene glycol isostearate, propylene glycol monooleate, propylene glycol dicaprylate/dicaprate, propylene glycol dioctanoate, propylene glycon caprylate/caprate, propylene glycol dilaurate, propylene glycol distearate, propylene glycol dicaprylate, propylene glycol dicaprate.

A sorbitan fatty acid ester for example sorbitan monolaurate, sorbitan monopalmitate, sorbitan monooleate, sorbitan monostearate, sorbitan trioleate, sorbitan sesquioleate, sorbitan tristearate, sorbitan monoisostearate, sorbitan sesquistearate.

Lower alcohol fatty acid esters for example ethyl oleate, isopropy myristate, isopropyl palmitate, ethyl linoleate, isopropyl linoleate.

Polyoxyethylene-polyoxypropylene block copolymers for example poloxamer 105, poloxamer 108, poloxamer 122, poloxamer 123, poloxamer 124, poloxamer 181, poloxamer 182, poloxamer 183, poloxamer 184, poloxamer 185, poloxamer 188, poloxamer 212, poloxamer 215, poloxamer 217, poloxamer 231, poloxamer 234, poloxamer 235, poloxamer 237, poloxamer 238, poloxamer 282, poloxamer 284, poloxamer 288, poloxamer 331, poloxamer 333, poloxamer 334, poloxamer 335, poloxamer 338, poloxamer 401, poloxamer 402, poloxamer 403, poloxamer 407.

Polyglycerised fatty acids for example polyglyceryl stearate, polyglyceryl oleate, polyglyceryl isostearate, polyglyceryl laurate, polyglyceryl ricinoleate, polyglyceryl linoleate, polyglyceryl pentaoleate, polyglyceryl dioleate, polyglyceryl distearate, polyglyceryl trioleate, polyglyceryl septaoleate, polyglyceryl tetraoleate, polyglyceryl decaisostearate, polyglyceryl decaoleate, polyglyceryl monooleate, dioleate, polyglyceryl polyricinoleate.

PEG alkyl ethers for example PEG oleyl ether, PEG lauryl ether, PEG cetyl ether, PEG stearyl ether.

PEG alkyl phenols for example PEG nonyl phenol, PEG octyl phenol ether.

Transesterification products of alcohol or polyalcohol with natural or hydrogenated oils for example PEG castor oil, PEG hydrogenated castor oil, PEG castor oil, PEG corn oil, PEG almond oil, PEG apricot kernel oil, PEG olive oil, PEG-6 peanut oil, PEG hydrogenated palm kernel oil, PEG palm kernel oil, PEG triolein, PEG corn glycerides, PEG almond glycerides, PEG trioleate, PEG caprylic/capric triglyceride, lauroyl macrogol glyceride, stearoyl macrogol glyceride, mono, di, tri, tetra esters of vegetable oils and sorbitol, pentaerythrityl tetraisostearate, pentaerythrityl distearate, pentaerythrityl tetraoleate, pentaerythrityl tetrastearate, pentaerythrityl tetracaprylate/tetracaprate, pentaerythrityl tetraoctanoate.

Oil-soluble vitamins for example vitamins A, D, E, K, and isomers, analogues, and derivatives thereof. The derivatives include, for example, organic acid esters of these oil-soluble vitamin substances, for example the esters of vitamin E or vitamin A with succinic acid. Derivatives of these vitamins include tocopheryl PEG-1000 succinate (Vitamin E TPGS) and other tocopheryl PEG succinate derivatives with various molecular weights of the PEG moiety, for example PEG 100-8000.

Sterols or sterol derivatives (e.g. esterified or etherified sterols as for example PEGylated sterols) for example cholesterol, sitosterol, lanosterol, PEG cholesterol ether, PEG cholestanol, phytosterol, PEG phytosterol.

Sugar esters for example sucrose distearate, sucrose distearate/monostearate, sucrose dipalmitate, sucrose monostearate, sucrose monopalmitate, sucrose monolaurate, alkyl glucoside, alkyl maltoside, alkyl maltotrioside, alkyl glycosides, derivatives and other sugar types: glucamides.

Carboxylates (in particular carboxylate esters) for example ether carboxylates, succinylated monoglycerides, sodium stearyl fumarate, stearoyl propylene glycol hydrogen succinated, mono/diacetylated tartaric acid esters of mono- and diglycerides, citric acid esters of mono-, diglycerides, glyceryl-lacto esters of fatty acids; acyl lactylates: lactylic esters of fatty acids, calcium/sodium stearoyl-2-lactylate calcium/sodium stearoyl lactylate, alginate salts, propylene glycol alginate.

A fatty acid monoglyceride, diglyceride or triglyceride or a combination thereof.

Examples of macrogol esters which are suitable for use in the present invention are macrogol esters of fatty acids having at least 6 carbon atoms and optionally at least 10 carbon atoms, and particularly of at least 12 carbon atoms; some fatty acids have no more than 22 carbon atoms, for example C₁₀-C₂₀, C₁₂-C₂₀ or C₁₅-C₂₀ fatty acids. The fatty acids may be saturated or unsaturated but are in particular saturated. To be mentioned are macrogol 25 cetostearyl ether (Cremophor® A25); macrogol 6 cetostearyl ether (Cremophor® A6); macrogol glycerol ricinoleate 35 (Cremophor® EL); macrogol-glycerol hydroxystearate 40 (Cremophor® RH 40); macrogol-15-hydroxystearate (Solutol® HS 15). Examples of macrogol ethers which are suitable for use in the present invention are macrogol ethers of fatty alcohols having at least 6 carbon atoms and optionally at least 10 carbon atoms, and particularly of at least 12 carbon atoms; some fatty alcohols have no more than 22 carbon atoms, for example C₁₀-C₂₀, C₁₂-C₂₀ or C₁₅-C₂₀ fatty alcohols. The fatty alcohols may be saturate or unsaturated but are in one embodiment saturated.

Examples of copolymers, which are suitable for use in the present invention are: pluronics(poloxamers); polyvinyl pyrollidone-polyvinylacetate (Plasdone S630); aminoalkyl methacrylate copolymer (Eudragit EPO); methacrylic acid-methyl methacrylate copolymer (Eudragit S100, L100); polycaprolactone-PEG; polycaprolactone-methoxy-PEG; poly(aspartic acid)-PEG; poly(benzyl-L-glutamate)-PEG; poly(D,L-lactide)methoxy-PEG; poly(benzyl-L-aspartate-PEG; or poly(L-lysine)-PEG

In a preferred embodiment the micelle-forming surfactant cis a macrogol ester, more preferably a macrogol ester that conforms to the European Pharmacopoeia monograph number 2052 macrogol-15-hydroxystearate, such as Kolliphor® HS 15 marketed by BASF.

### Anionic Surfactants

The surfactant (first and/or second surfactant) may be or comprise at least one anionic surfactant.

The surfactant (first and/or second surfactant) may be a fatty acid salt or bile salt for example sodium caproate, sodium caprylate, sodium caprate, sodium laurate, sodium myristate, sodium myristolate, sodium palmitate, sodium palmitoleate, sodium oleate, sodium ricinoleate, sodium linoleate, sodium linolenate, sodium stearate, sodium lauryl sulfate, sodium tetradecyl sulfate, sodium lauryl sarcosinate, sodium dioctyl sulfosuccinate; sodium cholate, sodium taurocholate, sodium glycocholate, sodium deoxycholate, sodium taurodeoxycholate, sodium glycodeoxycholate, sodium ursodeoxycholate, sodium chenodeoxycholate, sodium taurochenodeoxycholate, sodium glyco chenodeoxycholate, sodium cholylsarcosinate and sodium N-methyl taurocholate. Preferably the second surfactant is sodium lauryl sulphate.

Phospholipids for example egg/soy lecithin, cardiolipin, sphingomyelin, phosphatidylcholine, phosphatidyl ethanolamine, phosphatidic acid, phosphatidyl glycerol, phosphatidyl serine.

Phosphoric acid esters having the general formula RO-PO3-M+ where the R group is an ester forming group, e.g. an alkyl, alkenyl or aryl group optionally substituted by a PEG moiety through which the alkyl, alkenyl or aryl group is coupled to the phosphate moiety. R may be a residue of a long chain (e.g. >C9) alcohol or a phenol. Specific examples include diethanolammonium polyoxyethylene-10 oleyl ether phosphate, esterification products of fatty alcohols or fatty alcohol ethoxylates with phosphoric acid or anhydride.

Sulfates and sulfonates (in particular esters thereof) for example ethoxylated alkyl sulfates, alkyl benzene sulfones, α-olefin sulfonates, acyl isethionates, acyl taurates, alkly glyceryl ether sulfonates, octyl sulfosuccinate disodium, disodium undecylenamideo-MEA-sulfosuccinate, alkyl phosphates and alkyl ether phosphates.

### Cationic Surfactants

The surfactant (first and/or second surfactant) may be or comprise at least one cationic surfactant selected from the following cationic surfactants.

Hexadecyl triammonium bromide, dodecyl ammonium chloride, alkyl benzyldimethylammonium salts, diisobutyl phenoxyethoxydimethyl benzylammonium salts, alkylpyridinium salts; betains (trialkylglycine): lauryl betaine (N-lauryl,N,N-dimethylglycine); ethoxylated amines: polyoxyethylene-15 coconut amine, alkyl-amines/diamines/quaternary amines and alkyl ester.

Examples of amphiphilic polymers which are suitable for use in the present invention are: alkyl glucamides; fatty alcohol poly(ethoxyl)ates also known as polyethoxylated alkyl ethers; poly(ethoxyl)ated fatty acid esters (Myrj or Solutol); fatty amide polyethoxylate; fatty amine ethoxylate; alkylphenol ethoxylate; polyethoxylated sorbitan esters (polysorbates); polyethoxylated glycerides; or poly-glycerol esters.

### Emulsifiers

The surfactant may act as an emulsifier such surfactants include non-ionic emulsifiers, for example selected from: a mixture of triceteareth-4 phosphate, ethylene glycol palmitostearate and diethylene glycol palmitostearate (for example sold under the trade mark SEDFOS™ 75); sorbitan esters, e.g. sorbitan monooleate, sorbitan monolaurate, sorbitan monpalmitate, sorbitan monostearate (for example products sold under the trade mark Span®), PEG-8 beeswax e.g. sold under the trade mark Apifil®; a mixture of cetyl alcohol, ceteth-20 and steareth-20 (for example Emulcire™ 61 WL 2659); a mixture of PEG-6 stearate and PEG-32 stearate (for example Tefose® 1500); a mixture of PEG-6 palmitostearate, ethylene glycol palmitostearate, and PEG-32 palmitostearate (e.g. Tefose® 63); triglycerol diisostearate (for example products sold under the trade mark Plurol Diisostearique®); polyglyceryl-3 dioleate (for example products sold under the trade mark Plural® Oleique).

### Preferred First Surfactant

Preferably, the surfactant is a medium chain or long chain fatty acid mono- or di-glyceride or a combination thereof that does not comprise or is not a polyethyleneglycol ether or ester. Where the surfactant comprises a medium chain or long chain fatty acid mono- or di-glyceride or a combination thereof that does not comprise or is not a polyethyleneglycol ether or ester, the medium chain or long chain fatty acid mono- or di-glyceride or a combination thereof is substantially all of the surfactant. For example, the surfactant may comprise medium chain or long chain fatty acid mono- or di-glyceride or a combination thereof that does not comprise or is not a polyethyleneglycol ether or ester in an amount of greater than 80% of the surfactant, optionally greater than 85%, 90%, 95%, 97%, 98% or 99%. Suitably, the surfactant is substantially free of a triglyceride. For example, the surfactant may comprise less than 10%, 8%, 5%, 3%, 2% or 1% of a triglyceride.

A medium chain fatty acid mono-ester or di-ester comprises a fatty acid having 8 to 12 in chain carbon atoms. A long chain fatty acid mono-ester or di-ester comprises a fatty acid having at least 13 in chain carbon atoms, preferably 13 to 26 in chain carbon atoms. The long chain fatty acid may optionally have from 14 to 22 in chain carbon atoms or 16 to 20 in chain carbon atoms.

A mono-glyceride or di-glyceride surfactant may comprise one glycerol esterified to one fatty acid or one glycerol esterified to two fatty acids the fatty acids may be the same or different, ordinarily the fatty acids will be the same. The surfactant of the invention is a surfactant that does not comprise or is not a polyethyleneglycol ether or ester; by this it is meant that there is no polyethyleneglycol component bonded to the surfactant molecule by an ether or ester linkage. For example a pegylated fatty acid glyceride such as oleoyl macrogol-6 glycerides (commercially available as Labrafil M1944CS). It is possible that a commercial surfactant of the invention is supplied with a small amount of polyethyleneglycol (PEG) contained within the supplied surfactant composition. The use of such commercial formulations of surfactants which contain non-bonded PEG, put another way free PEG) are not excluded by the limitation that the surfactant does not comprise or is not a polyethyleneglycol ether or ester.

The surfactant may be or comprise a medium chain or long chain fatty acid mono- or di-glyceride or a combination thereof and may not comprise or may not be a polyethyleneglycol ether or ester, wherein the fatty acid ester is saturated or unsaturated. Preferably, the fatty acid is unsaturated. The unsaturated fatty acid may contain only one or only two double bonds.

Where the surfactant is a medium chain or long chain fatty acid di-glyceride (by which it is meant that there are two fatty acids esterified to a glycerol) the surfactant may comprise two fatty acids which are the same or different. For example the two fatty acids may both be unsaturated or may both be saturated. Alternatively, one of the two fatty acids may be saturated and the other fatty acid may be unsaturated.

Preferably the surfactant is a long chain mono- or di-glyceride or a combination thereof and does not comprise or is not a polyethyleneglycol ether or ester. A further preferred surfactant is a long chain mono- or di-glyceride or a combination thereof and does not comprise or is not a polyethyleneglycol ether or ester, wherein the fatty acid has a chain length of 13 to 22 carbon atoms, optionally 16 to 20 carbon atoms. In particular the fatty acid may have a chain length of 18 carbon atoms.

In an embodiment the surfactant is selected from: glyceryl monocaprate, glyceryl dicaprate, glyceryl monocaprylate, glyceryl dicaprylate, glyceryl caprate, glyceryl monocaprylate/caprate, glyceryl caprylate/caprate glyceryl dicaprylate/caprate, glyceryl monooleate/dioleate, glyceryl monooleate, glyceryl dioleate, glyceryl monostearate, glyceryl distearate, glyceryl monopalmitostearate, glyceryl dipalmitostearate, glyceryl monobehenate, glyceryl dibehenate, glycerol monolinoleate, glyceryl dilinoleate, polyglyceryl dioleate, propylene glycol monoheptanoate, and a combination thereof.

A preferred surfactant may be or comprise a surfactant selected from: glyceryl monocaprylate/caprate, glyceryl dicaprylate/caprate, glyceryl monooleate, glycerol monolinoleate, glyceryl dioleate, glyceryl monostearate, glyceryl distearate, glyceryl monopalmitostearate, glyceryl dipalmitostearate, glyceryl monobehenate, glyceryl dibehenate, glyceryl monolinoleate, glyceryl dilinoleate, polyglyceryl dioleate and a combination thereof.

Accordingly, there is provided a composition comprising cyclosporin, a hydrogel forming polymer matrix, a surfactant and an oil phase being dispersed in the hydrogel forming polymer matrix, wherein the surfactant may be or may comprise a surfactant selected from: glyceryl monocaprylate/caprate, glyceryl dicaprylate/caprate, glyceryl monooleate, glycerol monolinoleate, glyceryl dioleate, glyceryl monostearate, glyceryl distearate, glyceryl monopalmitostearate, glyceryl dipalmitostearate, glyceryl monobehenate, glyceryl dibehenate, glyceryl monolinoleate, glyceryl dilinoleate, polyglyceryl dioleate and a combination thereof.

The surfactant may comprise or be a surfactant selected from: glyceryl caprylate, glyceryl caprate, glyceryl monooleate, glyceryl dioleate, glycerol monolinoleate or a combination thereof.

A particularly preferred surfactant may be or comprise a surfactant selected from: glyceryl caprylate/caprate (Capmul MCM), glyceryl monooleate/dioleate (Capmul GMO-50) and glycerol monolinoleate (Maisine 35-1).

Optionally, the surfactant is not a mixture of glyceryl monostearate EP/NF and PEG-75 palmitostearate (for example Gelto™ 64). Suitably, the surfactant may not be or comprise a mixture of glyceryl monostearate.

### Preferred Second Surfactants.

Preferably the second surfactant is an anionic surfactant. For example, the second surfactant may be an alkyl sulphate, for example sodium dodecyl sulphate.

The second surfactant may be a fatty acid salt or bile salt for example sodium caproate, sodium caprylate, sodium caprate, sodium laurate, sodium myristate, sodium myristolate, sodium palmitate, sodium palmitoleate, sodium oleate, sodium ricinoleate, sodium linoleate, sodium linolenate, sodium stearate, sodium lauryl sulfate, sodium tetradecyl sulfate, sodium lauryl sarcosinate, sodium dioctyl sulfosuccinate; sodium cholate, sodium taurocholate, sodium glycocholate, sodium deoxycholate, sodium taurodeoxycholate, sodium glycodeoxycholate, sodium ursodeoxycholate, sodium chenodeoxycholate, sodium taurochenodeoxycholate, sodium glyco chenodeoxycholate, sodium cholylsarcosinate and sodium N-methyl taurocholate. Preferably the second surfactant is sodium lauryl sulphate.

### Other Excipients

The composition optionally contains one or more of the following additional substances or categories of substances. For example, the composition may contain a protectant such as, for example, a proteolytic enzyme inhibitor or a protector against acid degradation or both (e.g. an alkali for example sodium hydroxide); an adhesive entity such as, for example, a muco- or bio-adhesive; excipients to maximize solubility of the active ingredient; excipients to maximize permeability of the active ingredient in the GIT. Typical excipients for enhancing the permeability of the epithelial barrier include but are not limited to sodium caprate, sodium dodecanoate, sodium palmitate, SNAG, chitosan and derivatives thereof, fatty acids, fatty acid esters, polyethers, bile salts, phospholipids, alkyl polyglucosides, sugar esters, hydroxylase inhibitors, antioxidants (e.g. ascorbic acid) and/or nitric oxide donors. The preceding list is of particular interest to enhance permeability in the ileum.

To enhance permeability in the colon, typical excipients include, but not limited to sodium caprate, sodium dodecanoate, sodium palmitate, SNAG, chitosan and derivatives thereof, fatty acids, fatty acid esters, polyethers, bile salts, phospholipids, alkyl polyglucosides, hydroxylase inhibitors, antioxidants (optionally selected from curcuminoids, flavonoids, curcumin, beta-carotene, α-tocopherol, ascorbic acid, ascorbate, lazaroid, carvedilol, butylated hydroxytoluene, propyl gallate, hydralazine, carnosic acid, vitamin E, lecithin Ovolecithin (vitelin), vegilecithin, fumaric acid or citric acid) and/or nitric oxide donors, including nitric oxide donor groups covalently attached to various pharmaceutically active ingredients. The composition may further comprise excipients or other active pharmaceutical or other ingredients to enhance local tissue bioavailability in the GIT, such as the small intestine or colon, including efflux pump inhibitors, including, but not limited to PgP pump inhibitors (optionally selected from NSAIDs, cimetidine, omeprazole, Vitamin E TPGS, verapimil, quinidine, PSC833, amprenavir (APV), indinavir (IDV), nelfinavir (NFV), ritonavir (RTV) and saquinavir (SQV)), and metabolism inhibitors, including, but not limited to, cytochrome P450 inhibitors, optionally selected from: essential oils, cimetidine, surfactants (for example cremophor), oils, omeprazole, verapamil, ritonavir and carbamazepine as well as plant extracts, e.g., from citrus fruits. The composition may therefore further comprise a P450 inhibitor to further reduce metabolism of cyclosporin following administration of the composition. The P450 inhibitor may act to inhibit enteric and/or hepatic metabolism of the cyclosporin. The composition may further comprise a PgP inhibitor. Optionally the composition may comprise a P450 inhibitor and a PgP inhibitor.

The composition may further comprise excipients to enhance the therapeutic potential of an active ingredient, for example cyclosporin A or another immunosuppressant, throughout the gastrointestinal tract, including in the ileum and colon including, but not limited to absorption limiters, essential oils such as, for example, omega 3 oils, natural plant extracts such as, for example, neem, ion-exchange resins, bacteria degradable conjugation linkers such as, for example, azo bonds, polysaccharides such as, for example, amylose, guar gum, pectin, chitosan, inulin, cyclodextrins, chondroitin sulphate, dextrans, guar gum and locust bean gum, nuclear factor kappa B inhibitors, acids such as, for example, fumaric acid, citric acid and others, as well as modifications thereof.

The composition may further comprise excipients to reduce systemic side effects associated with absorption of certain active, for example cyclosporin or other immunosuppressants, in the GIT, such as the small intestine, including, but not limited to, antioxidants, such as, for example, curcuminoids, flavanoids or more specifically including curcumin, beta-carotene, α-tocopherol, ascorbate or lazaroid.

The composition may further or separately comprise antioxidants (such as, for example, ascorbic acid or BHT—butyl hydroxy toluene) taste-masking or photosensitive components or photoprotective components. Antioxidants may be incorporated in the aqueous phase (e.g. hydrophilic antioxidants) or in the disperse phase of the core (e.g. hydrophobic antioxidants such as, for example, vitamin E) for example up to 1% by weight, preferably between 0.01 and 0.50% by weight, more preferably between 0.10 to 0.20% by weight.

The composition may further comprise immune-enhancing nutrients such as vitamins A/B/C/E; carotenoids/beta-carotene and iron, manganese, selenium, zinc, especially when the composition contains an immunosuppressant, as in the case of an immunosuppressant targeted to the ileum and/or colon, e.g. the colon. Such nutrients may be present in composition, or if the composition has a coating, for example if it is the form of a bead, the nutrients may be included in the coating.

The composition may also include other well know excipients used in pharmaceutical compositions including colorants, taste masking agents, diluents, fillers, binders etc. The presence of such optional additional components will of course depend upon the particular dosage form adopted.

### Shape, Size and Geometry

The composition of the invention can be formed into a limitless number of shapes and sizes. In the section below describing the process for making the composition, various methods are given including pouring or introducing a fluid dispersion into a mould where it hardens or can be caused to harden. Thus the composition can be created in whichever form is desired by creating an appropriate mould (e.g. in the shape of a disc, pill or tablet). However, it is not essential to use a mould. For example, the composition may be formed into a sheet e.g. resulting from pouring a fluid dispersion onto a flat surface where it hardens or can be caused to harden.

Preferably, the composition may be in the form of spheres or spherical-like shapes made as described below. Preferably, the composition of the invention is in the form of substantially spherical, seamless minibeads. The absence of seams on the minibead surface is an advantage e.g. in further processing, for example coating, since it allows more consistent coating, flowability etc. The absence of seams on the minbeads also enhances consistency of dissolution of the beads.

The preferred size or diameter range of minibeads according to the invention can be chosen to avoid retention in the stomach upon oral administration of the minibeads. Larger dosage forms are retained for variable periods in the stomach and pass the pyloric sphincter only with food whereas smaller particles pass the pylorus independently of food. Selection of the appropriate size range (see below) thus makes the therapeutic effect post-dosing more consistent. Compared to a single large monolithic oral format such as, for example, a traditional compressed pill, a population of beads released into the GI tract (as foreseen by the dosage form of the present invention) permits greater intestinal lumen dispersion so enhancing absorption via exposure to greater epithelial area, and achieves greater topical coating in certain parts of the GI tract for example the colon). Reduction of residence time in the ileo-caecal junction is another potential advantage.

The composition of the invention is preferably monolithic meaning internally (i.e. cross-sectionally) homogeneous, excluding a possible thin skin of matrix material and excluding any coating layers.

The minibeads provided for by the formulation of the present invention generally range in diameter from 0.5 mm to 10 mm with the upper limit preferably 5 mm, e.g. 2.5 mm A particularly convenient upper limit is 2 mm or 1.7 mm. The lower limit can preferably be 1 mm, e.g. 1.2 mm, more preferably from 1.3 mm, most preferably from 1.4 mm. In one embodiment the diameter is from 0.5 to 2.5 mm, for example from 1 mm to 3 mm, 1 mm to 2 mm, 1.2 mm to 3 mm or 1.2 mm to 2 mm. The minibeads may have a diameter of no more than 2.5 mm, irrespective of their minimum size. The beads may have a diameter of no more than 2 mm, irrespective of their minimum size.

A minibead as described herein may have an aspect ratio of no more than 1.5, e.g. of no more than 1.3, for example of no more than 1.2 and, in particular, of from 1.1 to 1.5, 1.1 to 1.3 or, 1.1 to 1.2. A population of minibeads as described herein, e.g. at least 10 beads, may have an average aspect ratio of no more than 1.5, e.g. of no more than 1.3, for example of no more than 1.2 and, in particular, of from 1 to 1.5, 1 to 1.3 or 1 to 1.2. The aspect ratios mentioned in this paragraph optionally apply to coated minibeads and optionally apply to uncoated minibeads. Average aspect ratio is suitably determined for a population of minibeads, e.g. at least 10 minibeads, using a particle size analyser, for example an Eyecon™ particle characteriser of Innopharma Labs, Dublin 18, Ireland.

The minibeads of the disclosure may, therefore, have a size as disclosed above and an aspect ratio of from 1 to 1.5. The beads of the disclosure may have a size as disclosed above and an aspect ratio of no more than 1.3, for example of no more than 1.2 and, in particular, of from 1.1 to 1.5, 1.1 to 1.3 or, 1.1 to 1.2.

Bead size (diameter) may be measured by any suitable technique, for example microscopy, sieving, sedimentation, optical sensing zone method, electrical sensing zone method or laser light scattering. For the purposes of this specification, bead size is measured by analytical sieving in accordance with USP General Test <786> Method I (USP 24-NF 18, (U.S. Pharmacopeial Convention, Rockville, Md., 2000), pp. 1965-1967).

In embodiments, minibeads of the invention are monodisperse. In other embodiments, minibeads of the invention are not monodisperse. By “monodisperse” is meant that for a population of beads (e. g. at least 100, more preferably at least 1000) the minibeads have a coefficient of variation (CV) of their diameters of 35% or less, optionally 25% or less, for example 15% or less, such as e.g. of 10% or less and optionally of 8% or less, e.g. 5% or less. A particular class of polymer beads has a CV of 25% or less. CV when referred to in this specification is defined as 100 times (standard deviation) divided by average where “average” is mean particle diameter and standard deviation is standard deviation in particle size. Such a determination of CV is performable using a sieve.

The invention includes minibeads having a CV of 35% and a mean diameter of 1 mm to 2 mm, e.g. 1.5 mm. The invention also includes minibeads having a CV of 20% and a mean diameter of 1 mm to 2 mm, e.g. 1.5 mm, as well as minibeads having a CV of 10% and a mean diameter of 1 mm to 2 mm, e.g. 1.5 mm. In one class of embodiments, 90% of minibeads have a diameter of from 0.5 mm to 2.5 mm, e.g. of from 1 mm to 2 mm.

### Dosage Forms

The composition of the invention may be prepared as an orally administrable dosage form suitable for pharmaceutical use. In those embodiments where the formulation is in the form of a minibead, the present invention provides for a dosage form comprising a plurality of the minibeads for example as a capsule, a tablet, a sprinkle or a sachet. The minibeads may also be administered rectally or vaginally administered composition, for example as an enema or suppository. The composition, for example in the form of minibeads may be blended in a suitable medium to provide a suppository or enema compositions. Suitable media for suppositories and enemas are well known and include for example, a low melting point wax for a suppository or a suitable aqueous or oil based medium for an enema composition.

In embodiments the dosage form comprising a population of beads may be presented in a single unit dosage form e.g. contained in a single hard gel capsule which releases the beads e.g. in the stomach. Alternatively the beads may be presented in a sachet or other container which permits the beads to be sprinkled onto food or into a drink or to be administered via a feeding tube for example a naso-gastric tube or a duodenal feeding tube. Alternatively, the beads may be administered as a tablet for example if a population of beads is compressed into a single tablet as described below. Alternatively, the beads may be filled e.g. compressed into a specialist bottle cap or otherwise fill a space in a specialised bottle cap or other element of a sealed container (or container to be sealed) such that e.g. on twisting the bottle cap, the beads are released into a fluid or other contents of the bottle or vial such that the beads are disperse (or dissolve) with or without agitation in such contents. The fluid or other contents of the bottle or vial may optionally contain one of more additional active agent(s) to facilitate the convenient co-administration of the cyclosporin composition with other active agents. An example is the Smart Delivery Cap manufactured by Humana Pharma International (HPI) S.p.A, Milan, Italy. In embodiments comprising more than one population of minibeads the populations of mininbeads may be formulated into the same dosage form or may be formulated into separate dosages forms, the dosage forms optionally being the same or different.

The dosage form may be formulated in such a way so that the beads of the invention can be further developed to create a larger mass of beads e.g. via compression (with appropriate oil or powder-based binder and/or filler known to persons skilled in the art. The larger (e.g. compressed) mass may itself take a variety of shapes including pill shapes, tablet shapes, capsule shapes etc. A particular problem which this version of the bead embodiment solves is the “dead space” (above the settled particulate contents) and/or “void space” (between the particulate content elements) typically found in hard gel capsules filled with powders or pellets. In such pellet- or powder-filled capsules with dead/void space, a patient is required to swallow a larger capsule than would be necessary if the capsules contained no such dead space. The beads of this embodiment of the invention may readily be compressed into a capsule to adopt the inner form of whichever capsule or shell may be desired leaving much reduced, e.g. essentially no, dead/void space. Alternatively the dead or void space can be used to advantage by suspending beads in a vehicle such as, for example, an oil which may be inert or may have functional properties such as, for example, permeability enhancement or enhanced dissolution or may comprise an active ingredient being the same or different from any active ingredients in the bead. For example, hard gelatin or HPMC capsules may be filled with a liquid medium combined with uncoated and/or coated beads. The liquid medium may be one or more of the surfactant phase constituents described herein or it may be one or more surfactants. Particularly preferred but non-limiting examples are corn oil, sorbitane trioleate (sold under the trade mark SPAN 85), propylene glycol dicaprylocaprate (sold under the trade mark Labrafac), 2-(2-ethoxyethoxy)ethanol (sold under the trade mark Transcutol P) and polysorbate 80 (sold under the trade mark Tween 80).

In a representative embodiment the bead of the dosage form is prepared as described herein for example by mixing together at least the following materials: a hydrogel-forming polymer; an oil phase, a surfactant being or comprising a medium chain or long chain fatty acid mono- or di-glyceride or a combination thereof, wherein the surfactant does not comprise or is not a polyethyleneglycol ether or ester, and cyclosporin A, suitably cyclosporin A being dissolved in the oil phase, such as a liquid lipid to form a dispersion of the cyclosporin A in the hydrogel-forming polymer. The dispersion is immobilized within the solidified bead by ejection from a single orifice nozzle into a suitable cooling liquid. Following removal of the cooling liquid the bead is coated with a modified release coating (the second coating) (suitably with a sub-coat under the modified release coating), the coated bead is then optionally filled into a gelatin or HPMC capsule suitable for pharmaceutical use.

Suitably the dosage form is prepared as a unit dosage form containing from for oral administration comprising from 0.1 mg to 1000 mg, optionally from 1 mg to 500 mg, for example 10 mg to 300 mg, 15 mg to 300 mg, or 25 to 250 mg, suitably about 15 mg, about 25 mg, about 35 mg, about 50 mg, about 75 mg, about 100 mg, about 150 mg, about 180 mg, about 200 mg, about 210 mg or about 250 mg cyclosporin A

### Determination of Contents and Distribution of Formulations

The identity and/or distribution of one or more of the components of a composition according to the invention can be determined by any method known to those skilled in the art. The distribution of one or more components of a composition can, for example, be determined by near-infrared (NIR) chemical imaging technology. NIR chemical imaging technology can be used to generate images of the surface or cross section of a composition, for example a minibead. The image produced by this technique shows the distribution of one or more components of the composition. In addition to NIR chemical imaging technology, the distribution of one or more components of a composition such as minibead, for example, be determined by time-of-flight secondary ion mass spectrometry (ToFSIMS). ToFSIMS imaging can reveal the distribution of one or more components within the composition. The images produced by ToFSIMS analysis or NIR analysis can show the distribution of components across a surface of the composition or a cross section of the composition. The methods described in this paragraph are applicable, for example, to composition comprising a polymer matrix, e.g. a dried, colloid, solution or dispersion.

### Manufacturing Processes

Various methods may be used to prepare the formulations of the invention.

In those embodiments where the formulation comprises an active ingredient in a water-insoluble polymer matrix, a basic method for making the core is to mix a fluid form of the matrix material, for example a hydrogel forming polymer matrix material (e.g. poly(amides), poly(amino-acids), hyaluronic acid; lipoproteins; poly(esters), poly(orthoesters), poly(urethanes) or poly(acrylamides), poly(glycolic acid), poly(lactic acid) and corresponding co-polymers (poly(lactide-co-glycolide acid; PLGA); siloxane, polysiloxane; dimethylsiloxane/methylvinylsiloxane copolymer; poly(dimethylsiloxane/methylvinylsiloxane/methylhydrogensiloxane) dimethylvinyl or trimethyl copolymer; silicone polymers; alkyl silicone; silica, aluminium silicate, calcium silicate, aluminium magnesium silicate, magnesium silicate, diatomaceous silica etc as described more generally elsewhere herein), with an active ingredient to form a mixture that may take the form of a suspension, solution or a colloid. The mixture is processed to form a composition or a core. For example the composition may be shaped into the desired form using a molding or hot-melt extrusion process to form beads.

Methods for preparing a composition, or in certain embodiments a core, comprising an NFAT inhibitor, an oil phase and a water-soluble polymer matrix are described below. Generally these cores are coated. The composition also optionally comprises a surfactant.

Generally, the manufacturing processes described herein comprise mixing of liquid(s). Such mixing processes must be performed at temperatures at which the substances to be mixed in the liquid state are in liquid form. For example, thermoreversible gelling agents must be mixed at a temperature where they are in the liquid state, for example at a temperature of 50 to 75° C., for example 50 to 70° C., or 55-75° C., e.g. 60-70° C. and in particular embodiments about 55° C. or 65° C. in the case of mixing formulations comprising aqueous gelatin. Similarly other components of the formulation may need to be heated to melt the component for example waxes or surfactants which may be used in the disperse phase.

The composition or the core comprising oil phase, hydrogel-forming polymer and an NFAT inhibitor (e.g. cyclosporin), and optionally a surfactant, as disclosed herein may be made by mixing materials comprising for example water, a hydrogel-forming polymer and optionally a second surfactant to form an aqueous continuous phase, and mixing a disperse phase. At least one of the aqueous phase and the disperse phase comprises an NFAT inhibitor (e.g. cyclosporin), the an NFAT inhibitor may be dissolved in the phase which contains it, for example both phases may be a clear liquid before they are mixed together. Preferably, the disperse phase (the oil phase) may comprise the NFAT inhibitor (e.g. cyclosporin), (for example a disperse phase comprising an oil, an optional solvent, the NFAT inhibitor and a first surfactant) with the aqueous phase to form a colloid. The colloid may have the form of an emulsion or microemulsion wherein the disperse phase is dispersed in the aqueous continuous phase. This colloid may optionally represent the liquid composition of the invention. In order to prepare the composition of the invention or the core, the hydrogel-forming polymer is then caused or allowed to gel to form a hydrogel forming polymer matrix. Suitably, the process includes formulating or processing the composition into a desired form, e.g. a bead (also termed a minibead), which forming process may comprise moulding but preferably comprises ejecting the aqueous colloid through a single orifice nozzle to form droplets which are caused or allowed to pass into a cooling medium, e.g. a water-immiscible cooling liquid, in which the droplets cool to form for e.g. beads.

The mixing of the materials may comprise mixing an aqueous premix (or aqueous phase or continuous phase) and a disperse phase premix (e.g. oil phase premix), wherein the aqueous premix comprises water and water-soluble substances whilst the disperse phase premix may comprise a vehicle containing an NFAT inhibitor (e.g. cyclosporin) and the surfactant. The vehicle may be a hydrophobic liquid, for example a liquid lipid, or it may be or comprise a material, for example a surfactant, for forming self-assembly structures. In particular, a disperse phase premix may comprise cyclosporin A, the first surfactant, an oil and other oil soluble components for example an optional solvent. The premixes may contain one or more surfactants suitable for the phase they are to form, as previously mentioned, for example the aqueous premix may comprise a second surfactant.

The aqueous premix comprises, or usually consists of, a solution in water of water-soluble constituents, namely the hydrogel-forming polymer, water-soluble excipient(s) and optionally the NFAT inhibitor (preferably when the NFAT inhibitor is water soluble). The aqueous premix may include a plasticiser for the hydrogel-forming polymer, as described elsewhere in this specification. The aqueous premix may include a second surfactant, e.g. to increase polymer viscosity and improve emulsification and thereby help prevent precipitation of active agent during processing. SDS is an example of such a surfactant. In any event, the constituents of the aqueous premix may be agitated for a period sufficient to dissolve/melt the components, for example, from 1 hour to 12 hours to form the completed aqueous premix.

The disperse phase pre-mix may comprise the first surfactant and the NFAT inhibitor (preferably where the NFAT inhibitor is soluble in the disperse phase, for example oil soluble) as a dispersion or preferably a solution in a vehicle (for example an oil phase) as described above, for example in a liquid comprising an oil or in a liquid comprising component(s) of self-assembly structures. For example an oil phase pre-mix may therefore be a liquid lipid, for example a medium chain triglyceride (MCT) formulation, the medium chain triglyceride(s) being one or more triglycerides of at least one fatty acid selected from C₆-C₁₂ fatty acids, and cyclosporin A and the surfactant comprising or being a medium or long chain fatty acid mono- or di-glyceride. Suitably an oil phase pre-mix is stirred at ambient temperature to form a solution of the NFAT inhibitor in the oil and surfactant. In some embodiments, the components of the oil phase premix are mixed (or otherwise agitated) for a period of, for example, 10 minutes to 3 hours to form the premix.

The two premixes may be combined and agitated, for example for a period of a few seconds to an hour, for example from 30 seconds to 1 hour, suitably 5 mins to an hour, to form a dispersion of the disperse phase in an aqueous hydrogel-forming polymer to form the liquid composition of the invention. The dispersion may then be further processed to form the composition or a core. The two premixes may be combined into the dispersion by agitation in a mixing vessel; they may additionally or alternatively be combined in a continuous flow mixer.

The basic method for making a composition or core comprising the NFAT inhibitor and hydrogel-forming polymer matrix, therefore, is to mix a liquid form (preferably a solution) of the hydrogel-forming polymer (or mixture of polymers) with the NFAT inhibitor, the surfactant (to avoid any ambiguity the first surfactant) and the oil phase (and any other disperse phase components) to form a dispersion in the polymer, which later in the process forms a hydrogel. The method normally comprises mixing together an aqueous polymer phase premix and a disperse phase premix. Taking account of the final composition required (as described elsewhere herein), the disperse phase pre-mix and the liquid hydrogel-forming polymer (i.e. the solution or suspension of hydrogel-forming polymer, the aqueous phase) may be mixed in a weight ratio of from 1:1 to 1:10, particularly 1:4 to 1:9, e.g. 1:5 to 1:7. In general, only gentle stirring of the components is required using a magnetic or mechanical system, e.g. overhead stirrer, as would be familiar to a person skilled in the art to achieve a dispersion of the disperse phase in the aqueous phase to form a colloid (which may be in the form of for example an emulsion or micro emulsion in which the aqueous hydrogel is the continuous phase). Continuous stirring is preferred. Mixing may also be achieved using an in-line mixing system. Any appropriate laboratory stirring apparatus or industrial scale mixer may be utilized for this purpose for example the Magnetic Stirrer (manufactured by Stuart) or Overhead Stirrer (by KNF or Fisher). It is preferred to set up the equipment in such a way as to minimise evaporation of contents such as, for example, water. In one embodiment of the process of the invention, it is preferred to utilise a closed system for stirring in order to achieve this aim. In-line mixing may be particularly suitable for closed system processing. Suitably mixing of the two components takes place at a temperature of 50 to 70° C., or 55-75° C., e.g. 60-70° C.

The mixing of the two phases results in a colloid wherein the aqueous hydrogel-forming polymer is an aqueous continuous phase and the component(s) not soluble in the aqueous phase are a disperse phase. The colloid may have the form of an emulsion or microemulsion.

The colloid is formed by combining of the disperse phase premix with the liquid aqueous phase with stirring as described above. The resultant colloidal dispersion then has the formulation of a solidified core described above but with liquid water still present in the core formulation.

By use of the term “dry”, it is not sought to imply that a drying step is necessary to produce the dry core (although this is not excluded) rather that the solid or solidified aqueous external phase is substantially free of water or free of available water. Solidification of the aqueous phase (external phase) may have arisen through various means including chemically (e.g. by cross-linking) or physically (e.g. by cooling or heating). In this respect, the term “aqueous phase” is nevertheless employed in this document to denote the external (continuous) phase of the core even though water, in certain embodiments, is largely absent from (or trapped within the cross-linked matrix of) the core. The external phase of the core is however water-soluble and dissolves in aqueous media.

Means for gelling or solidifying compositions of the present invention are known in the art. For example, the reader is directed to WO2015/067763, in particular pages 81 to 88. The whole contents of this document is incorporated herein by reference.

It will be appreciated, therefore, that the invention includes a process for manufacturing a composition of the invention or a core comprising an NFAT inhibitor, a surfactant, and an oil phase in a hydrogel forming polymer matrix, which process comprises: forming an aqueous premix which comprises water and water soluble/dispersible materials (including therefore a hydrogel-forming polymer) and a disperse phase premix (e.g. an oil phase premix) which comprises the oil phase, the NFAT inhibitor and the surfactant optionally other excipients (e.g. oil(s) and oil soluble/dispersible materials), and combining the two premixes to form a colloid (disperse phase) within an aqueous phase comprising the hydrogel-forming polymer. The colloid may then be formed into a shaped unit, for example a bead to provide the core comprising the active ingredient. More particularly the manufacture of a composition or core as defined above may comprise:

(i) forming an aqueous phase pre-mix comprising a solution in water of water-soluble constituents (e.g. of a hydrogel-forming polymer, any water-soluble excipient(s), as described elsewhere herein);
(ii) forming a disperse phase pre-mix typically comprising a dispersion or preferably a solution of NFAT inhibitor, in a liquid lipid, and the surfactant, optionally together with other disperse phase constituents (e.g. surfactant, solvents etc as described elsewhere herein);
(iii) mixing the aqueous phase pre-mix (i) and the disperse phase pre-mix (ii) to form a colloid;
(iv) ejecting the colloid through a nozzle to form droplets;
(v) causing or allowing the a hydrogel-forming polymer to gel or solidify to form a water soluble polymer matrix; and
(vi) drying the solid.

The manufacture of a liquid composition of the invention may comprise:

(i) forming an aqueous phase pre-mix comprising a solution in water of water-soluble constituents (e.g. of a hydrogel-forming polymer, any water-soluble excipient(s), as described elsewhere herein);

(ii) forming a disperse phase pre-mix typically comprising a dispersion or preferably a solution of NFAT inhibitor, in a liquid lipid, and the first surfactant, optionally together with other disperse phase constituents (e.g. surfactant, solvents etc as described elsewhere herein); and

(iii) mixing the aqueous phase pre-mix (i) and the disperse phase pre-mix (ii) to form a colloid.

Some manufacturing processes comprise steps (A) to (D) below or, alternatively, a manufacturing process may comprise a single one or any combination of steps (A) to (D).

(A) Exemplary Preparation of Aqueous Phase:

Aqueous phase components are added to water, e.g. purified water, under agitation e.g. sonication or stirring. The temperature is gradually increased, for example to 60-70° C. and in particular 65° C., to achieve complete dissolution of the solids. The aqueous phase components include a hydrogel-forming polymer, e.g. gelatin or agar and optionally one or more other excipients, for example D-sorbitol (a plasticiser) and surfactant (for example SDS). Possible aqueous phase components are described elsewhere herein.

The gelatin may be Type A gelatin. In some less preferred implementations, the gelatin is Type B. The gelatin may have a Bloom strength of 125-300, optionally of 200-300, for example of 225-300, and in particular 275. The components of the aqueous phase may be agitated for a period of, for example, from 1 hour to 12 hours to complete preparation of the aqueous phase (aqueous premix).

(B) Exemplary Preparation of Disperse Phase:

The NFAT inhibitor is mixed with the surfactant, an oil and other disperse phase components (for example a co-solvent) under agitation e.g. sonication or stirring, suitably at ambient temperature to disperse or preferably dissolve the active ingredient.

(C) Exemplary Mixing of the Two Phases

The aqueous phase and the disperse phase are mixed. The two phases may be mixed in a desired weight; for example, the weight ratio of disperse phase to aqueous phase may be from 1:1 to 1:10, e.g. from 1:4 to 1:9 and optionally from 1:5 to 1:8 such as about 1:5 or about 1:7. The resulting colloid is agitated, e.g. sonicated or stirred, at a temperature of 60-70° C. and in particular 65° C., to achieve a homogeneous dispersion, then the homogenous dispersion is formed into beads. In particular, the homogenous dispersion is ejected through a single orifice nozzle to form droplets which fall into a cooling medium. The nozzle is suitably vibrated to facilitate droplet formation. The nozzle may be vibrated at a frequency of 2-200 Hz and optionally 15-50 Hz.

The cooling medium may for example be air or an oil; the oil is suitably physiologically acceptable as, for example, in the case of medium chain triglycerides e.g. Miglyol 810N. The cooling medium may be at a cooling temperature often of less than 15° C., for example of less than 10° C. but above 0° C. In some embodiments the cooling temperature is 8-10° C. The nozzle size (diameter) is typically from 0.5 to 7.5 mm, e.g. from 0.5 to 5 mm and optionally from 0.5 to 4 mm. In some embodiments, the nozzle diameter is from 1 to 5 mm for example from 2 to 5 mm, and optionally from 3 to 4 mm, and in particular may be 3.4 mm. The nozzle diameter may be from 1 to 2 mm.

The flow rate through a 3.4 mm nozzle or through a 1.5 mm nozzle is 5 to 35 g/min and optionally 10 to 20 g/min and for nozzles of different sizes may be adjusted suitably for the nozzle area.

(D) Exemplary Processing of Beads

Cooled beads are recovered, for example they may be recovered from cooling oil after a residence time of 15-60 minutes, for example after approximately 30 minutes. Beads recovered from a cooling liquid (e.g. oil) may be centrifuged to eliminate excess cooling liquid, and then dried. Suitably, drying is carried out at room temperature, for example from 15-40° C. and optionally from 20-35° C. The drying may be performed in a drum drier, for example for a period from 6 to 24 hours, e.g. of about 12 hours in the case of beads dried at room temperature. The dried beads may be washed, suitably with a volatile non-aqueous liquid at least partially miscible with water, e.g. they may be washed with ethyl acetate. The washed beads may be dried at room temperature, for example from 15-25° C. and optionally from 20-25° C. The drying may be performed in a drum drier, for example for a period from 6 to 48 hours, e.g. of about 24 hours in the case of beads dried at room temperature. Drying may be achieved by any suitable means, for example using a drum dryer, suitably under vacuum; or by simply passing warm air through the batch of beads, or by fluidising the beads in a suitable equipment with warm air, for example if a fluid bed dryer. Following drying, the beads are passed through a 1 to 10 mm, optionally 2 to 5 mm to remove oversized beads and then through a sieve with a pore size of 0.5 to 9 mm optionally 1 to 4 mm to remove undersized beads.

It can be appreciated that it is possible to recycle the beads that are rejected by the sieving process.

As a further aspect of the invention there is provided a formulation obtainable by (having the characteristic of) any of the processes described herein. It is to be understood that the processes described herein may therefore be used to provide any of the specific cores described in embodiments herein by dispersing the appropriate components which form the disperse phase of the core in the appropriate components which form the aqueous continuous matrix phase of the core.

The preceding paragraphs describe the formation of uncoated compositions or cores. The composition may comprise a coating. Cores may be coated. The composition or the core may be coated with a subcoat and/or coated with a second coating (also referred to as a modified release coating or outer coat). Suitable sub coats and modified release coatings (second coating or outer coat) are any of those described herein and any of the first coating (for the subcoat) or the second coating (for the modified release coating). The coating(s) may be applied using well known methods, for example spray coating as described below to give the desired sub coat and modified release coating weight gains.

With regard to one of the methods described above (ejection of emulsion through an optionally vibrating nozzle) with two concentric orifices (centre and outer), the outer fluid may form a coating (outside the bead) as described herein. The Spherex machine manufactured by Freund (see U.S. Pat. No. 5,882,680 to Freund) is preferably used (the entire contents of this patent is incorporated herein by reference). Other similar ejection or extrusion apparatus may also be used, for example the ejection apparatus described hereinbefore.

Use of the Spherex machine achieves very high monodispersity. For example, in a typical 100 g, batch 97 g of beads were between 1.4 to 2 mm diameter or between 1 and 2 mm. Desired size ranges can be achieved by methods known in the art for rejecting/screening different sized particles. For example, it is possible to reject/screen out the larger/smaller beads by passing a batch first through e.g. a 2 mm mesh and subsequently through a 1.4 mm mesh.

The 1.4 to 2 mm diameter range is a good size if it is desired to spray coat the beads (if smaller, the spray of the coating machine may bypass the bead; if too large, the beads may be harder to fluidise, which is necessary to achieve consistent coating).

### Coating Process

The coating process can be carried out by any suitable means such as, for example, by use of a coating machine which applies a solution of a polymer coat (as described above in particular) to the formulation. Polymers for coating are either provided by the manufacturer in ready-made solutions for direct use or can be made up before use following manufacturers' instructions. The coating process can be as described under the “Coating Process” heading in WO2015/067763, The contents of which is incorporated herein by reference.

Coating is suitably carried out using a fluid bed coating system such as a Wurster column to apply the coating(s) to the composition or the core. Appropriate coating machines are known to persons skilled in the art and include, for example, a perforated pan or fluidized-based system for example the GLATT, Vector (e.g. CF 360 EX), ACCELACOTA, Diosna, O'Hara and/or HICOATER processing equipment. To be mentioned is the MFL/01 Fluid Bed Coater (Freund) used in the “Bottom Spray” configuration.

Typical coating conditions are as follows:

| Process Parameter | Values |
|---|---|
| Fluidising airflow (m3/h) | 20-60 (preferably 30-60) |
| Inlet air temperature (° C.) | 20-65 |
| Exhaust air temperature (° C.) | 20-42 |
| Product temperature (° C.) | 20-45 (preferably 40 to 42) |
| Atomizing air pressure (bar) | Up to 1.4 e.g. 0.8-1.2 |
| Spray rate (g/min) | 2-10 and 3-25 RPM |

Suitably the coating is applied as a solution or dispersion of the polymers (and other components) of the coating. Generally the coatings are applied as an aqueous, solution or dispersion, although other solvent systems may be used if required. The coating dispersion is applied to the composition or the core as a spray in the fluid bed coater to give the required coating weight gain. Generally the coating process is carried out at a temperature which maintains the cores at a temperature of from 35 to 45° C., preferably 40 to 42° C.

After applying the coating, the composition may be dried, for example by drying at 40 to 45° C.

The invention further provides a product having the characteristics of a composition obtained as described herein, a product defined in terms of its characteristics being defined by the characteristics of the composition to the exclusion of the method by which it was made.

As mentioned herein the processes described may be used to provide any of the composition described in the various embodiments herein. By way of example there is provided a composition of the invention comprising a core and a first coating comprising a water-soluble cellulose ether or a water soluble derivative of a cellulose ether and/or a second coating comprising a delayed release polymer wherein the core comprises a hydrogel-forming polymer matrix comprising gelatin, an NFAT inhibitor, medium chain mono-di- and/or tri-glycerides, a first surfactant being or comprising a medium chain or long chain fatty acid mono- or di-glyceride or a combination thereof that does not comprise or is not a polyethyleneglycol ether or ester, a co-solvent and optionally a second surfactant, the core having the characteristics of a core obtained by the process comprising steps (i) to (vi) described above for forming the core, wherein the aqueous phase pre-mix in step (i) of the process comprises gelatin and optionally a second surfactant (suitably an anionic surfactant), and the oil phase pre-mix in step (ii) of the process comprises medium chain mono-di- or tri-glycerides, hydrophobic active ingredient, surfactant (suitably a non-ionic surfactant) and cosolvent; and the wherein the core is optionally coated with a first coating comprising a water-soluble cellulose ether or a water soluble derivative of a cellulose ether and/or a second coating comprising a delayed release polymer; wherein the coatings are any of those described herein. Accordingly, the process may produce a composition as described above comprising a first coating and/or a second coating. The process may additionally produce a composition comprising a first coating and a second coating being outside the first coating.

In a particular embodiment the composition or the core is in the form of a solid colloid, the colloid comprising a continuous phase and a disperse phase, wherein

the disperse phase is or comprises:

cyclosporin;

a medium chain mono-, di- and/or tri-glyceride, for example a medium chain triglyceride, particularly caprylic/capric triglyceride;

a medium- or long-chain mono- or di-glyceride, particularly glyceryl monooleate/dioleate; and

a co-solvent (for example 2-(ethoxyethoxy)ethanol);

and wherein the continuous phase is or comprises:

a hydrogel-forming polymer matrix which is or comprises a hydrocolloid selected from carrageenan, gelatin, agar and pectin, or a combination thereof optionally selected from gelatin and agar or a combination thereof, more particularly the polymer of the a hydrogel-forming polymer matrix is or comprises gelatin;

a plasticiser, optionally a plasticiser selected from glycerin, a polyol for example sorbitol, polyethylene glycol and triethyl citrate or a mixture thereof, particularly sorbitol; and

an anionic surfactant, for example at least one surfactant selected from fatty acid salts, alkyl sulphates and bile salts, particularly an alkyl sulphate, for example sodium dodecyl sulphate.

In another particular embodiment the composition or the core is in the form of a solid colloid, the colloid comprising a continuous phase and a disperse phase, wherein

the disperse phase is or comprises:

cyclosporin;

a medium chain mono-, di- and/or tri-glyceride, for example a medium chain triglyceride, particularly caprylic/capric triglyceride;

a non-ionic surfactant (for example a polyethoxylated castor oil (e.g. Kolliphor EL) medium- or long-chain mono- or di-glyceride, particularly glyceryl monooleate/dioleate; and

a co-solvent (for example 2-(ethoxyethoxy)ethanol);

and wherein the continuous phase is or comprises:

a hydrogel-forming polymer matrix which is or comprises a hydrocolloid selected from carrageenan, gelatin, agar and pectin, or a combination thereof optionally selected from gelatin and agar or a combination thereof, more particularly the polymer of the a hydrogel-forming polymer matrix is or comprises gelatin;

a plasticiser, optionally a plasticiser selected from glycerin, a polyol for example sorbitol, polyethylene glycol and triethyl citrate or a mixture thereof, particularly sorbitol; and

an anionic surfactant, for example at least one surfactant selected from fatty acid salts, alkyl sulphates and bile salts, particularly an alkyl sulphate, for example sodium dodecyl sulphate.

In a the above embodiment the core comprises a hydrogel forming polymer matrix comprising gelatin in an amount of 300 to 700 mg/g, the core further comprising cyclosporin A, medium chain mono-, di- or tri-glycerides (for example a medium chain triglyceride, particularly caprylic/capric triglyceride) in an amount of 20 to 200 mg/g, and the core further comprises the following components:

co-solvent (for example 2-(ethoxyethoxy)ethanol) in an amount of 150 to 250 mg/g;

non-ionic surfactant in an amount of 80 to 200 mg/g; and

anionic surfactant in an amount of 15 to 50 mg/g,

wherein weights are based upon the dry weight of the core.

Suitably in the embodiment of the above paragraph the cyclosporin A may be present in an amount of 60 to 150 mg/g, for example 80 to 120 mg/g or particularly 80 to 100 mg/g. The non-ionic and anionic surfactants are as defined herein, for example an anionic surfactant selected from alkyl sulfates, carboxylates or phospholipids (particularly SDS); or a non-ionic surfactant selected from sorbitan-based surfactants, PEG-fatty acids, or glyceryl fatty acids or poloxamers. A particular non-ionic surfactant is a polyethoxylated castor oil (for example Kolliphor™ EL).

In a further specific embodiment the disperse phase comprises:

cyclosporin in an amount of 60-180 mg/g;

caprylic/capric triglyceride in an amount of 40-80 mg/g;

2-(2-ethoxyethoxy)ethanol in an amount of 100-200 mg/g; and

glyceryl monooleate and/or glyceryl dioleate in an amount of 100-150 mg/g, wherein weights are based upon the dry weight of the composition.

The oil phase or disperse phase may comprise:

cyclosporin in an amount of 120-360 mg/g;

caprylic/capric triglyceride in an amount of 80-160 mg/g; 2-(2-ethoxyethoxy) ethanol in an amount of 200-400 mg/g; and

glyceryl monooleate and/or glyceryl dioleate in an amount of 200-300 mg/g,

wherein the weights are based on the weight of the wet composition.

The liquid composition may comprise an oil phase comprising:

cyclosporin in an amount of 20-60 mg/g;

caprylic/capric triglyceride in an amount of 13-27 mg/g;

2-(2-ethoxyethoxy) ethanol in an amount of 50-70 mg/g; and

glyceryl monooleate and/or glyceryl dioleate in an amount of 30-55 mg/g,

wherein weights are based upon the wet weight of the composition, i.e. the liquid composition, optionally wherein the oil phase to aqueous phase ratio may be 1:5.

In an embodiment the aqueous phase or continuous phase comprises a hydrogel-forming polymer matrix comprising gelatin in an amount of 300 to 700 mg/g, and SDS in an amount of 15-50 mg/g, wherein weights are based upon the dry weight of the composition.

In an embodiment the aqueous phase may comprise a hydrogel-forming polymer matrix comprising gelatin in an amount of 120 to 280 mg/g and SDS in an amount of 6-20 mg/g wherein the weights are based upon the weight of the aqueous phase. The aqueous phase may comprise a hydrogel-forming polymer matrix comprising gelatin in an amount of 100 to 230 mg/g and SDS in an amount of 5-16 mg/g, wherein the weights are based on the weight of the composition, i.e. the liquid composition, optionally wherein the oil phase to aqueous phase ratio may be 1:5.

Suitably in the embodiment of the immediately preceding two paragraphs the cyclosporin may be present in an amount of 90 to 140 mg/g, for example of 60 to 150 mg/g, 80 to 120 mg/g or particularly 80 to 100 mg/g. The anionic surfactants are as defined herein, for example an anionic surfactant selected from alkyl sulphates, carboxylates or phospholipids (particularly SDS).

The composition or the cores described herein comprising hydrogel-forming polymer matrix may be coated as described herein. A particular coating for these embodiments is a coating comprising:

a first coating (sub-coating) which is or comprises a water-soluble cellulose ether, particularly hydroxypropylmethyl cellulose;

a second coating outside the first coating which is or comprises a modified release coating, particularly a pH independent modified release coating, more especially a coating comprising ethyl cellulose (e.g. Surelease) still more particularly a coating comprising ethyl cellulose and a water-soluble polysaccharide such as pectin (e.g. a Surelease-pectin coating as described herein); and wherein

the first coating is present in an amount corresponding to a weight gain due to the first coating in a range selected from: (i) from 8% to 12%, for example about 10%; or (ii) from 4% to 6%, for example about 5% by weight based upon the weight of the formulation prior to applying the first coating; and wherein

the second coating is present in an amount corresponding to a weight gain of the formulation due to the second coating selected from (a) from 10% to 12%, for example about 11% or about 11.5%; or (b) from 16% to 18%, for example about 17% by weight based upon the weight of the formulation prior to applying the second coating.

Equally, the composition or the cores described above comprising hydrogel-forming polymer matrix may be coated with a coating comprising:

a second coating which is or comprises a modified release coating, particularly a pH independent modified release coating, more especially a coating comprising ethyl cellulose (e.g. Surelease) still more particularly a coating comprising ethyl cellulose and a water-soluble polysaccharide such as pectin (e.g. a Surelease-pectin coating as described herein); and wherein

the second coating is present in an amount corresponding to a weight gain of the formulation due to the second coating selected from (a) from 10% to 12%, for example about 11% or about 11.5%; or (b) from 16% to 18%, for example about 17% by weight based upon the weight of the formulation prior to applying the second coating.

In the cores described herein to which the following characteristics are applicable, e.g. in the immediately preceding paragraph, the following characteristics may be present:

gelatin may be present in an amount of 300 to 700 mg/g;

the medium chain mono-, di- or tri-glycerides (for example caprylic/capric triglyceride) may be present in an amount of 20 to 200 mg/g;

co-solvent (for example 2-(ethoxyethoxy)ethanol) may be present in an amount of 150 to 250 mg/g;

non-ionic surfactant (for example sorbitan-based surfactants, PEG-fatty acids, or glyceryl fatty acids or poloxamers or particularly a polyethoxylated castor oil for example Kolliphor EL) may be present in an amount of 80 to 200 mg/g;

anionic surfactant (for example, alkyl sulphates, carboxylates or phospholipids (particularly SDS)) may be present in an amount of 15 to 50 mg/g; and

cyclosporin A, may be present in an amount of from 60 to 180 mg/g, suitably 60 to 150 mg/g, 90 to 150 mg/g, or 80 to 100 mg/g, for example 81 to 98 mg/g;

wherein all weights are based upon the dry weight of the core before coating.

The composition may comprise or the core may be coated with a first coating (sub-coating) which is or comprises a water-soluble compound selected from cellulose ethers and their derivatives, particularly hydroxypropylmethyl cellulose; the first coating being present in an amount corresponding to a weight gain due to the first coating in a range selected from: (i) from 8% to 12%, for example about 10%; or (ii) from 4% to 6%, for example about 5% by weight based upon the weight of the core prior to applying the first coating. The first coating may have a modified release coating (or second coating) applied to it.

Preferably, any modified release coating (second coating), especially in the embodiments of the immediately preceding paragraphs, is or comprises a pH independent modified release coating, more especially the second coating may be a modified release coating comprising ethyl cellulose (eg Surelease) still more particularly a modified release coating comprising ethyl cellulose and a water-soluble polysaccharide, pectin (e.g. a Surelease-pectin coating as described herein); and wherein the modified release coating is present in an amount corresponding to a weight gain of the formulation due to the second coating selected from (a) from 10% to 12%, for example about 11% or about 11.5%; or (b) from 16% to 18%, for example about 17% by weight based upon the weight of the formulation prior to applying the second coating.

In addition the process to form a composition of the invention may comprise the steps of mixing a first population and a second population, wherein

the first population has a coating that is or comprises a water-soluble cellulose ether but having no outer coating, e.g. as described herein; and

the second population has a first coating that is or comprises a water-soluble cellulose ether and a second coating that is or comprises a delayed release coating, for example as described herein e.g. a coating that is or comprises a delayed release polymer.

### Therapy Mediated by NFAT-Activated T Cells

The therapy mediated by NFAT activated T cells may be any of the therapies described herein, including but not limited to:
a bi-specific antibody selected from: blinatumomab MEHD7945A, ABT-122, ABT-981, SAR156597, MM-111, IMCgp100, R05520985, XmAb5871, COVA322, ALX-0761, AFM13, AFM11, MEDI-565, Ertumaxomab, MGD006, MGD007, LY3164530 and AMv-564;
a high affinity Tcell receptor T cell selected from NY-ESO-1 TCR 1, HPV-16 E6 TCR1, HPV-16 E6 TCR, MAGE A3/A6 TCR1, MAGE A3 TCR1, SSX2 TCR1, NY-ESO TCR, MAGE-A-10 TCR, BPX-701 and ATTCK20;
an autologous CAR-T selected from CD19 CAR1, KTE-C19 CAR, EGFRvIII CAR, JCAR015, JCAR017, JCAR014, BPX-401, CBM-C19.1, CAR-T CD19, CTL109, JCAR018, JCAR023 JTCR016, a CAR-T directed to MUC16, a CAR-T directed to ROR1, BPX-601, bb2121, CAR-T CD30, CAR-T EGFR and CART-meso;

an allogenic CAR-T Therapy selected from UCART19, UCART123, UCART38, UCARTCS1 and EBV-CTL;

a checkpoint inhibitor selected from an anti-PD-1/anti-PD-L1 inhibitor, for example REGN2810, Opdivo, Keytruda, MED14736, MPDL3280A and PDR001 (PDR1), an antibody targeting lymphocyte-activation gene 3 (LAG3; CD223), for example LAG525, an anti-CTLA-4 receptor inhibitor selected from: ipilimumab and tremlimumab, and an anti-TIM-3 receptor inhibitors for example MBG453.

The therapy mediated by NFAT activated T cells may be a single therapy or two or more such therapies, suitably any two or more of the therapies described herein. For example, the therapy comprises two or more therapies selected from a bispecific T cell engager, a chimeric antigen receptor therapy and a checkpoint clockade therapy, for example wherein the therapy comprises a checkpoint blockade therapy and one or more therapy selected from a bispecific T cell engager and a chimeric antigen receptor therapy. For example the therapy mediated by NFAT activated T cells may comprise a PD1 checkpoint inhibitor (for example Medi4736 or Keytruda (pembrolizumab)) and one or more CAR T therapy, more specifically a combination of a PD1 checkpoint inhibitor (for example Medi4736 or Keytruda (pembrolizumab)) and one or more CD19 CART therapy, for example a CAR T selected from CD19 CAR1, KTE-C19 CAR, JCAR015, JCAR017, JCAR014, BPX-401 and CTL109. A further specific combination is Medi4736 and a CAR T selected from JCAR014, JCAR015 and JCAR017. Another combination is a bispecific T-cell engaging therapy, for example blinatumomab and a CAR T, for example any of the CAR T therapies described herein. Yet another combination may comprise a checkpoint inhibitor with a HIF antagonist or an IDO inhibitor. Another agent to use in combination with a NFAT-activated T cells of the invention is CpG oligodeoxynucleatides.

The therapy mediated by NFAT activated T cells may, or combinations thereof, may also be used together with one or more immune-oncology adjuvant therapies described herein.

### Applications

The composition of the invention may advantageously be used for oral delivery pharmaceutically active ingredients by virtue of the enhanced dissolution profiles achieved.

By maintenance of effectiveness of the therapy will be understood that T cells expressing functional, constitutive or activated NFAT are retained in sufficient amount systemically, outside the GI tract whereby their desired beneficial therapeutic effect is maintained. The present invention may modulate the systemic bioavailability of NFAT inhibitors to allow the (“on target”) effectiveness of the NFAT Tcells while controlling any negative (“off target”) effects that are driven by NFAT.

An aim of certain embodiments of the present invention is to reduce local and systemic inflammation, for example reduce the level of cytokines, particularly pro-inflammatory cytokines. Accordingly, the invention contemplates a composition that is capable of reducing or modulating local cytokine levels and systemic cytokine levels. Alternatively, the invention may reduce local cytokine levels in the gastrointestinal tract but not effect systemic cytokine levels and/or systemic lymphocyte (optionally T cell or NK cell) levels. In an embodiment the aforementioned features are mediated by a coating as defined elsewhere in the present application. For example, local gastrointestinal reduction in cytokine levels could be achieved with a composition of the present invention with a delayed release polymer coating.

Alternatively, the invention may reduce local cytokine and chemokine levels released variously by immune cells, including but not limited to T-lymphocytes, B-lymphocytes, antigen presenting cells, eosinophils, neutrophils and macrophages, in the gastrointestinal tract but not effect systemic cytokine levels and/or systemic lymphocyte (optionally T cell or NK cell) levels. In an embodiment the aforementioned features are mediated by a coating as defined elsewhere in the present application. For example, local gastrointestinal reduction in cytokine and chemokine levels could be achieved with a composition of the present invention with a delayed release polymer coating.

The compositions of the invention include modified release compositions which comprise cyclosporin A and a modified release coating, for example comprising a pH independent polymer, to target cyclosporin release to the lower intestine. Such compositions result in low systemic exposure to cyclosporin A, whilst providing high levels of cyclosporin A in the lower GI tract, particularly in the colon. Such compositions release the cyclosporin A in an active form for example as a solution, which provides enhanced absorption of cyclosporin A in the local tissue of the lower GI tract. When the composition is used in the form of minibeads, the minibeads are advantageously dispersed along large sections of the GI tract following oral administration and are therefore expected provide a more uniform exposure to cyclosporin to large sections of for example the colon.

Accordingly the modified release compositions according to the invention comprising cyclosporin for local treatment of the lower GI tract are expected to be useful in the treatment or prevention of a condition of the GIT. In particular the composition of the invention may comprise cyclosporin A and/or another immunosuppressant and be useful in the prevention or treatment of inflammatory conditions affecting the lower GI tract, particularly conditions affecting the colon.

The composition of the invention is suitably administered orally. The dose required will vary depending upon the specific condition being treated and the stage of the condition. In the case of compositions containing cyclosporin A, the composition will generally be administered to provide a dose of cyclosporin A of from 0.1 to 100 mg, for example a dose of 1 to 500 mg or particularly a dose of 25 to 250 mg cyclosporin A. The composition is suitably administered as a single daily dose.

Anti-cancer agents which may be suitable for use with the composition comprising an NFAT inhibitor or with the therapy mediated by NFAT activated T cells include, but are not limited to one or more agents selected from:

(i) antiproliferative/antineoplastic drugs and combinations thereof, such as alkylating agents (for example cis-platin, oxaliplatin, carboplatin, cyclophosphamide, nitrogen mustard, uracil mustard, bendamustin, melphalan, chlorambucil, chlormethine, busulphan, temozolamide, nitrosoureas, ifosamide, melphalan, pipobroman, triethylene-melamine, triethylenethiophoporamine, carmustine, lomustine, stroptozocin and dacarbazine); antimetabolites (for example gemcitabine and antifolates such as fluoropyrimidines like 5-fluorouracil and tegafur, raltitrexed, methotrexate, pemetrexed, cytosine arabinoside, floxuridine, cytarabine, 6-mercaptopurine, 6-thioguanine, fludarabine phosphate, pentostatine, and gemcitabine and hydroxyurea); antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors); proteasome inhibitors, for example carfilzomib and bortezomib; interferon therapy; and topoisomerase inhibitors (for example epipodophyllotoxins like etoposide and teniposide, amsacrine, topotecan, irinotecan, mitoxantrone and camptothecin); bleomcin, dactinomycin, daunorubicin, doxorubicin, epirubicin, idarubicin, ara-C, paclitaxel (Taxol™), nabpaclitaxel, docetaxel, mithramycin, deoxyco-formycin, mitomycin-C, L-asparaginase, interferons (especially IFN-alpha), etoposide, teniposide, DNA-demethylating agents, (for example, azacitidine or decitabine); and histone de-acetylase (HDAC) inhibitors (for example vorinostat, MS-275, panobinostat, romidepsin, valproic acid, mocetinostat (MGCD0103) and pracinostat SB939);
(ii) cytostatic agents such as antiestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride; and navelbene, CPT-II, anastrazole, letrazole, capecitabine, reloxafme, cyclophosphamide, ifosamide, and droloxafine;
(iii) anti-invasion agents, for example dasatinib and bosutinib (SKI-606), and metalloproteinase inhibitors, inhibitors of urokinase plasminogen activator receptor function or antibodies to Heparanase;
(iv) inhibitors of growth factor function: for example such inhibitors include growth factor antibodies and growth factor receptor antibodies, for example the anti-erbB2 antibody trastuzumab [Herceptin™] the anti-EGFR antibody panitumumab, the anti-erbB1 antibody cetuximab, tyrosine kinase inhibitors, for example inhibitors of the epidermal growth factor family (for example EGFR family tyrosine kinase inhibitors such as gefitinib, erlotinib, 6-acrylamido-N-(3-chloro-4-fluorophenyl)-7-(3-morpholinopropoxy)-quinazolin-4-amine (CI 1033), erbB2 tyrosine kinase inhibitors such as lapatinib) and antibodies to costimulatory molecules such as CTLA-4, 4-IBB and PD-I, or antibodies to cytokines (IL-10, TGF-beta); inhibitors of the hepatocyte growth factor family; inhibitors of the insulin growth factor family; modulators of protein regulators of cell apoptosis (for example Bcl-2 inhibitors); inhibitors of the platelet-derived growth factor family such as imatinib and/or nilotinib (AMN107); inhibitors of serine/threonine kinases (for example Ras/Raf signalling inhibitors such as farnesyl transferase inhibitors, for example sorafenib, tipifarnib and lonafarnib), inhibitors of cell signalling through MEK and/or AKT kinases, c-kit inhibitors, abl kinase inhibitors, PI3 kinase inhibitors, Plt3 kinase inhibitors, CSF-1R kinase inhibitors, IGF receptor, kinase inhibitors; aurora kinase inhibitors and cyclin dependent kinase inhibitors such as CDK2 and/or CDK4 inhibitors; and CCR2, CCR4 or CCR6 antagonists;
(v) antiangiogenic agents such as those which inhibit the effects of vascular endothelial growth factor, [for example the anti-vascular endothelial cell growth factor antibody bevacizumab (Avastin™)]; thalidomide; lenalidomide; and for example, a VEGF receptor tyrosine kinase inhibitor such as vandetanib, vatalanib, sunitinib, axitinib and pazopanib;
(vi) gene therapy approaches, including for example approaches to replace aberrant genes such as aberrant p53 or aberrant BRCA1 or BRCA2;
(vii) immunotherapy approaches, including for example antibody therapy such as alemtuzumab, rituximab, ibritumomab tiuxetan (Zevalin®) and ofatumumab; interferons such as interferon α; interleukins such as IL-2 (aldesleukin); interleukin inhibitors for example IRAK4 inhibitors; cancer vaccines including prophylactic and treatment vaccines such as HPV vaccines, for example Gardasil, Cervarix, Oncophage and Sipuleucel-T (Provenge); gp100; dendritic cell-based vaccines (such as Ad.p53 DC); toll-like receptor modulators for example TLR-7 or TLR-9 agonists; and
(viii) cytotoxic agents for example fludaribine (fludara), cladribine, pentostatin (Nipent™);

Optionally, the anti-cancer agents are not therapies mediated by NFAT activated T cells.

Immuno-Oncology Adjunct Therapies

The therapy mediated by NFAT may be used together with one or more adjunct therapies which potentiate or enhance the effect of the therapy. Accordingly the therapies described herein may be used together with one or more adjunct therapy. For example the therapy mediated by NFAT activated T cells may be used with an adjunct therapy selected from one or more of:

an adenosine A2A receptor inhibitor, for example HTL-1071;

anti CEACAM1 antibodies, for example CM-24, a humanized IgG4 mAb targeting carcinoembryonic antigen (CEA)-related cell adhesion molecule 1 (CEACAM1; CD66a);

a BRAF inhibitor, particularly when used with a checkpoint inhibitor), for example Tafinlar;

TGF beta receptor kinase inhibitors (particularly when used together with a checkpoint inhibitor);

A MAP kinase inhibitor, for example Mekinist (small molecule inhibitor of MAP kinase kinase 1 (MAP2K1; MEK1) and MEK2);

A STING (Stimulator of interferon genes) agonist, for example MIW815 (STING agonist);

an activator of NK cells, for example α-galactosylceramide (particularly when used together with a checkpoint inhibitor); and

a pro-inflammatory cytokine, for example IL-2, INFγ, GM-CSF, IL-7, IL-12, IL-15, IL-18 and IL-21;

or a combination of two or more thereof.

### Examples

### Example 1: Preparation of a Liquid Composition of the Invention

An aqueous phase was prepared by mixing sodium dodecyl sulphate (SDS) and D-sorbitol with purified water under constant stirring. Gelatin was then added to this solution and gentle heat was applied to approximately 60-70° C. to achieve complete melting of gelatin. The composition of the aqueous phase is shown in Table 1 below.

TABLE 1

| Component | w/w % |
|---|---|
| water | 79.6 |
| SDS | 1.3 |
| Sorbitol | 2.0 |
| Gelatin | 17.1 |

An oil phase was prepared by mixing together 2-(2-ethoxyethoxy)ethanol (Transcutol HP), glyceryl monooleate/dioleate (Capmul GMO-50) and capric/caprylic triglyceride (Miglyol 810) with stirring at room temperature to form a solution. Ciclosporin A was added and mixed until a clear solution was obtained. The composition of the oil phase is shown below in Table 2.

TABLE 2

| Component | w/w % |
|---|---|
| Cyclosporin A | 24.5 |
| Miglyol 810 N | 12.5 |
| Transcutol HP | 37.0 |
| Capmul GMO-50 | 26 |

The oil phase was mixed with the heated aqueous phase in a ratio of approximately 1:5 (oil phase: aqueous phase). The resulting mixture was stirred at 60-70° C., 250-350 rpm using a magnetic stirrer to achieve homogeneity.

### Example 2: Preparation of a Minibead

A minibead as described herein may be a composition of the invention. Alternatively the minibead may be a core. The minibead was generally prepared by forming a minibead according to the following procedure

The composition or core in the form of seamless minibeads were prepared using Spherex process as follows.

An aqueous phase and oil phase mixture was prepared following the procedure described in Example 1.

The mixture was then fed (via temperature controlled tubing) through a vibrating nozzle, with a single nozzle outlet with a diameter of 3 mm. Seamless minibeads were formed as the solution flowed through the vibrating nozzle into a cooling chamber of constantly flowing medium chain triglyceride (Miglyol 810) cooling oil at a temperature of 10° C.

The minibeads were removed from the cooling oil and placed in a centrifuge to remove the excess oil. Following centrifugation, a first drying step was initiated with a set refrigerator temperature of 10° C. and the heater temperature of 20° C. The dryer was rotated at 15 RPM. When the beads were observed to be freely rotating in the drying drum, they were considered to be dry.

The minibeads were washed with ethyl acetate and then dried for a further 24 h under the same drying conditions as those mentioned above in the first drying step. The dried minibeads were then sieved to remove oversize and undersize beads resulting in cores 1 mm-2 mm in diameter. This procedure provided cores with the composition shown in Table 3, the values being the weight percent of the total weight for each component.

TABLE 3

| Component | w/w % |
|---|---|
| Cyclosporin A | 12.1 |
| Miglyol 810 N | 6.2 |
| Transcutol HP | 18.3 |
| Capmul GMO-50 | 12.9 |
| SDS | 3.2 |
| Sorbitol | 4.9 |
| Gelatin | 42.4 |

### Example 3: Preparation of a Minibead with an Overcoat of Ethylcellulose

A minibead coated with Opadry, the first coating (also referred to as a subcoat), was produced following the procedure in Example 3. The minibead produced by the procedure of Example 2 was then coated with an overcoat (also referred to as a second coating herein) of Surelease® (an ethylcellulose dispersion).

The Surelease® overcoat was applied by the following procedure. Surelease® was slowly added to a stainless steel vessel and mixed to provide the required coating suspension of Surelease® for the overcoat. The resulting coating suspension was then applied onto the surface of the minibeads by loading the minibeads into a fluid bed coater (Wurster column) and coating with the suspension. The processing parameters, such as inlet air temperature and inlet air volume, were adjusted to keep the minibead temperature between 40° C. and 42° C. until the required coating weight gain was reached. The over-coated minibeads were then dried in the coater for an hour at 40-45° C.

The minibead was coated with a 13% weight gain of Surelease®.

The minibead with an overcoat has the composition shown in Table 4.

TABLE 4

| Component | w/w % |
|---|---|
| Cyclosporin A | 10.7 |
| Miglyol 810 N | 5.6 |
| Transcutol HP | 16.2 |
| Capmul GMO-50 | 11.4 |
| SDS | 2.8 |
| Sorbitol | 4.3 |
| Gelatin | 37.5 |
| Surelease | 11.5 |

### Example 4: Mouse Model of Undesirable Effects Induced by Infused T-Cells

The following mouse strain was used: NSG or NOD scid gamma (NOD.Cg-Prkdc^{scid} II2rg^{tm1WjI}/SzJ) (Jackson Labs, Bar Harbour, Me., USA). NSG is a strain of inbred laboratory mice, among the most immunodeficient described to date. NSG mice lack mature T cells, B cells, and natural killer (NK) cells. NSG mice are also deficient in multiple cytokine signaling pathways, and they have many defects in innate immunity. The compound immunodeficiencies in NSG mice permit the engraftment of a wide range of primary human cells, and enable sophisticated modeling of many areas of human biology and disease All mice were housed according to Dept. of Health (Ireland) guidelines and used with ethical approval under the terms of AE19124/P002 project authorisation from HPRA. Sample sizes for animal experiments were determined by statistical power calculation using SISA. SISA software is online at http://home.clara.net/sisa/power.htm.

Human Peripheral Blood Mononuclear Cell (PBMC) Isolation

Whole blood buffy coat packs, which contained red blood cells, white blood cells and platelets, were supplied by the Irish Blood Transfusion Service (IBTS) at St. James's Hospital, Dublin. PBMC were isolated from whole blood by density gradient centrifugation. The contents of buffy coat packs were diluted 1 in 2 with phosphate buffered saline (PBS) (Oxoid Ltd., Basingstoke, Hampshire, England). 25 ml diluted blood was carefully layered on top of 15 ml lymphoprep (Axis-Shield PoC AS, Oslo, Norway) in a 50 ml centrifugation tube (Sarstedt). Tubes were centrifuged at 2400 rpm for 25 min at room temperature with no brake and low acceleration. After centrifugation, the white buffy coat layer containing PBMC was removed into a new sterile 50 ml tube, leaving red blood cells and remaining plasma behind. Collected PBMC were centrifuged at 1800 rpm for 10 min at 4° C. with brake and acceleration at high settings. Supernatant was removed and the PBMC pellet was washed in 20 ml of PBS and centrifuged at 1500 rpm for 5 min at 4° C. for a total of two times. Remaining red blood cells were lysed using 5 ml 1× red blood cell lysis buffer (Biolegend, London, UK) for 5 min. 25 ml of complete RPMI (cRPMI) (RPMI 1640 (Sigma-Aldrich) supplemented with 10% (v/v) heat inactivated FBS, 50 U/ml penicillin (Sigma-Aldrich), 50 μg/ml streptomycin (Sigma-Aldrich), 2 mM L-glutamine (Sigma-Aldich) and 0.1% (v/v) 2-mercaptoethanol (Gibco)) was added to quench lysis. PBMC were centrifuged at 1000 rpm for 10 min at 4° C. to remove platelets. The PBMC pellet was resuspended in 25 ml of cRPMI and counted.

Administration of Human PBMC to Mouse

NOD.Cg-Prkdc^{scid}IL2^{tmIWjI}/Szj (NOD-Scid IL-2rγnull) (NSG) mice were exposed to a conditioning dose of 2.4 Gray (Gy) of whole body gamma irradiation. Freshly isolated human PBMC were administered by intravenous injection to the tail vein using a 27 gauge needle and a 1 ml syringe between 4 h but no longer than 24 h following irradiation. Before infusion, PBMC were washed three times with sterile PBS. From previous studies (Tobin et al. 2013), the optimum dose for PBMC was found to be 8.0×10⁵ g-1. Therefore 8.0×10⁵ g-1 was used for the present study. The mice were examined to determine whether they had weight loss, ruffled fur, hunched posture. Animals were returned to their cages where they were monitored closely for the first hour and at regular intervals thereafter for any signs of distress or ill health. Animals were weighed daily and weight loss was documented accordingly. Any animals which displayed greater than 15% total body weight loss were sacrificed humanely. In addition, an animal welfare score sheet was utilized throughout the study.

Intravenous Administration of Human MSC or PBMC

Before infusion, human PBMC or MSC were washed three times with sterile PBS. PBMC were administered to mice at 8.0×10⁵ g⁻¹ and MSC were administered at 5.7×10⁴ g⁻¹. PBMC or MSC were delivered to the tail vein using a 27 gauge needle and a 1 ml syringe. Each mouse received a total of 0.3 ml. PBMC were given on day 0 while MSC were given on day 7. Following i.v injection, animals were returned to their cages where they were monitored as above. F

Preparation and Administration of Cyclosporin Ormulations

Cyclosporin compositions of Example 2 and Example 3. Immediate release beads (Example 2) had a 10.87% loading of CsA (109 μg/mg). For each of these beads the average weight was in the range of 2-3 mg and the resultant active pharmacological ingredient (API) was 220-330 μ/mg per bead. Colonic release beads (Example 3) of CsA had a 10% loading of CsA (100 μg/mg) with an API of between 250-350 μg per bead. Each bead was weighed prior to administration to ensure correct dosage (25 mg/kg). Administration was carried out by oral gavage. Briefly, each bead was loaded at the end of a feeding needle (Vet Tech, Cheshire, UK)(Company, town, Country) with a syringe containing 200 μl PBS connected to it. Mice were carefully scruffed and the feeding needle was inserted into the mouth of the mouse. The feeding needle was carefully guided down the oesaphagus, where the beads were released with the aid of 200 μl PBS in a syringe. The Neoral® formulation of CsA was in the form of a 100 mg tablet. The CsA solution was removed from the inside of the tablet by needle (18 G) and 5 ml syringe and collected into a 50 ml tube. Prior to administration the Neoral® was diluted in PBS to yield a 25 mg/kg dose and 300 μl was delivered by oral gavage as described above. The Sandimmun® formulation of CsA was in the form of a 50 mg/ml injectable solution. The CsA solution was diluted in PBS to yield a 25 mg/kg dose prior to administration. Sandimmune® was delivered to the tail vein using a 27 gauge needle and a 1 ml syringe. Each mouse received a total of 0.3 ml. Following each procedure, animals were returned to their cages where they were monitored as above.

Mice were administered with the following combinations of immediate release (IR) and colonic release (CR) minibeads.

| Composition | Beads administered |
|---|---|
| A | 1 IR |
| B | 1 IR and 1 CR |
| C | 1 IR and 2 CR |
| D | 1 CR |
| E | 2 CR |

In addition to the mice groups that were treated with Compositions A to E described above, there were also mice groups that were administered with Neoral and Sandimmune as described above. There were also control groups of mice administered with PBS without any PBMC (called “PBS” in the results) and a group administered with PBMC but no cyclosporin, the untreated group (called “PBMC only” in the results).

Cellular and Cytokine Analysis from Mice

Isolation of Human Splenocytes from Mice

Spleens were removed aseptically from mice into a 50 ml tube containing cRPMI supplemented with 10% (v/v) heat inactivated FBS, 50 U/ml penicillium, 50 μg/ml streptomycin and 2 mM L-glutamine (Table 2.1). Spleens were homogenised through a 70 μm filter into a fresh 50 ml tube using a sterile plunger and the isolated splenocytes were then suspended in 10 ml cRPMI containing 0.1% v/v 2-mercaptoethanol (Invitrogen-Gibco). This homogenate was centrifuged at 300 g for 5 min and resuspended in 1 ml of red blood cell lysis buffer solution (BioLegend) for 10 min at room temperature. 2 ml of medium was added to the suspension to neutralise the lysis solution which was then centrifuged at 600 g for 5 min. Supernatant was removed and the cells were then resuspended in fresh cRPMI and counted. The cells were resuspended for FACS analysis (Section 2.10.1 or Section 2.10.2).

Cytokine Analysis from Splenic Cell Cultures

Spleens were removed from mice as described above and single cell suspensions were prepared. Cells were seeded at 2×10⁵ per well in a 96 well round bottom plate and cultured in cRPMI. Cells were unstimulated or stimulated with 10 μg/ml Phorbal 12-myrisate 13-acetate (Sigma-Aldrich) and 10 μg/ml ionomycin (Sigma-Aldrich). Supernatants were harvested after 72 hour for detection of IL-1β, IL-2, IL-6, IL-17, IL-23 and IFNγ. Cytokines in supernatants were detected by ELISA.

All ELISAs were carried out according to manufacturer's instructions (R & D Systems). Specific capture antibodies (human IFNγ, IL1β, IL2, IL6, IL17, or IL23) in PBS were added to 96 well microtitre plates (NUNC) and incubated overnight at room temperature. Plates were then washed 3 times in wash buffer (PBS supplememnted with 0.05% v/v Tween 20) and then incubated in blocking solution (PBS supplemented with 1% w/v BSA) for a minimum of 1 h. Plates were then washed and incubated with 100 μl/well of sample supernatant or corresponding cytokine standard for 2 h at room temperature. After washing, plates were incubated with specific detection antibodies for a further 2 h at room temperature. Plates were washed again and incubated with 100 μl/well of streptavidin horseradish peroxidase (HRP) (R & D Systems) conjugate diluted 1/40 in specific reagent diluent (Tris buffered solution (TBS) (Sigma-Aldrich) supplemented with BSA) for 20 min. After washing, plates were incubated with 100 μl/well of tetramethylbenzidine (TMB) substrate (Sigma-Aldrich) for 20 min at room temperature out of direct light. The reaction was stopped after 20 min by adding 50 μl/well of 1 M H₂SO₄. The absorbance (optical density (O.D)) of the samples and standards were measured at 450 nm for all ELISA using a miroplateplate reader (BioTek EL800) with Gen5 Data Analysis Software. The cytokine concentration of each sample was determined by comparison to the standard curve of known cytokine concentrations using Graph pad Prism5 software.

Isolation of Human Cells from the Liver or Lungs of Mice

Livers and lungs were removed aseptically from mice into a 50 ml tube containing cRPMI (Table 2.1). Tissues were homogenised through a 70 μm filter into a fresh 50 ml tube using a sterile plunger and the isolated cells were then suspended in 25 ml cRPMI. This homogenate was layered over 15 ml lymphoprep density gradient (Axis-Shield) and centrifuged at 2400 rpm for 25 min with no brake and low acceleration. The interface was collected by suction into a fresh labelled 50 ml tube. The interface was washed twice with 25 mls PBS and centrifuged at 300 g for 5 min. Supernatant was removed and the cells were resuspended for flow cytometry analysis as described below.

Isolation of Human Cells from the GI Tract of Mice

Small intestines and colons were removed aseptically from mice into a 15 ml tube containing cRPMI (Table 2.1). The tissue samples were placed in 10 ml digestion solution (20 U/ml DNase I (Roche Diagnostics, Germany), 300 U/ml collagenase from Clostridium histolyticum (Sigma-Aldrich) and PBS) at 37° C. under constant horizontal shaking at 300 rpm. After 1 hour of digestion, the homogenates were passed through a 70 μm filter into a fresh 50 ml tube using a sterile plunger and the isolated cells were then suspended in 25 ml cRPMI and centrifuged at 1500 rpm for 10 min. The cells were resuspended in 8 mls of 40% Percoll (Sigma-Alrdich), overlayed onto 4 mls of 80% Percoll (Sigma-Aldrich) and centrifuged at 2200 rpm for 20 min with no brake and low acceleration. The interface was removed by suction and transfered to a new tube. 15 ml of PBS was added and the interface was centrifuged at 1400 rpm for 8 min at 4° C. The supernatant was removed and the cells were resuspended for flow cytometry as described below.

Cytokine Analysis from the GI Tract of Mice

The small intestine and colon was removed from mice as described above where a section was immediately snap frozen and stored at −80° C. Tissues were thawed and gut contents were removed. The tissues were chopped finely and homogenised using an Ultra-Turrax homogeniser (Germany) in 1 ml of chilled homogenisation buffer (PBS: 2% heat inactivated FBS supplemented with protease inhibitor cocktail (Roche)). The homogenate was microcentrifuged at 13,000 rpm for 15 min at 4° C. The supernatant was removed and stored at −20° C. The protein concentration of GI tract extracts were determined by Bradford assay (Section 2.6.5). Protein extracts were analysed for IL-1β, IL-2, IL-6, IL-17, IL-23 and IFNγ. Cytokines in supernatants were detected by ELISA as described above.

Analysis of Human PBMC In Vitro and In Vivo by Flow Cytometry

Detection of Cytokine Production by Human Cells

TNFα and IFNγ were analsyed intracellularly by flow cytometry. Briefly, PBMC recovered from coculture assays or in vivo studies, were washed in 150 μl FACS buffer and centrifuged at 950 rpm for 5 min in 96 well v bottomed plates. PBMC were labelled with CD45 PercP, CD4 APC and CD8 PE or corresponding isotype control antibodies for 15 min at 4° C. The cells were washed twice in 150 μl FACS buffer, centrifuged at 950 rpm for 5 min and fixed with 100 μl fix/permeabilisation buffer (eBioscience) for 1 hour or overnight. The cells were then permeabilised with 200 μl permeabilisation buffer (eBioscience), washed with 150 μl FACS buffer and blocked using 3 μl 2% rat serum for 15 minutes. The cells were labelled with TNFα, IFNγ or isotype control antibodies and left at 4° C. for 1 hour. Samples were washed twice with 150 μl FACS buffer, resuspended in counting beads (3×10⁵/ml) and analysed by flow cytometry (Accuri C6 flow cytometer, BD Biosciences) using CFlowPlus software (BD Biosciences).

Intracellular Staining of Cells to Detect FoxP3 Expression

FoxP3 expression was analysed intracellularly using a FoxP3 staining kit (eBioscience). Briefly, PBMC recovered from coculture assays or in vivo studies, were washed in 150 μl FACS buffer and centrifuged at 950 rpm for 5 min. PBMC were labelled with CD4 APC and CD25 PE or corresponding isotype control antibodies for 15 min at 4° C. The cells were washed twice in 150 μl FACS buffer, centrifuged at 950 rpm for 5 min and fixed with 100 μl fix/permeabilisation buffer (eBioscience) for 1 hour. The cells were then permeabilised with 200 μl permeabilisation buffer (eBioscience), washed with 150 μl FACS buffer and blocked using 3 μl 2% rat serum for 15 minutes. The cells were labelled with FoxP3 or isotype control antibodies and left at 4° C. for 1 hour or overnight. Samples were washed twice with 150 μl FACS buffer, resuspended in counting beads (3×10⁵/ml) and analysed by flow cytometry (Accuri C6 flow cytometer, BD Biosciences) using CFlowPlus software (BD Biosciences).

Histology Tissue Preparation

The lungs, liver, spleen and small intestine were harvested from experimental mice at day 13 and fixed in 10% (v/v) neutral buffered formalin for at least 24 hours. Samples were transferred to 70% ethanol for a further 24 hours. Samples were processed for histology using an automated processor (Shandon Pathcentre, Runcorn, UK) which immersed the tissues in fixatives and sequential dehydration solutions including ethanol (70%, 80%, 95%×2, 100%×3) and xylene (×2) (Sigma-Aldrich). After processing, tissues were embedded in paraffin wax using a Shandon Histocentre 2 (Shandon) and left to set at 4° C. overnight. A Shandon Finesse 325 microtome (Thermo-Shandon, Waltham, Mass., USA) was used to cut 5 μm sections of each tissue. Sections were placed in cold water before being transferred to a hot water bath (42° C.) to remove any folding of the sections. Tissue sections were placed onto microscope slides (VWR) and left to air dry overnight. Samples were then stained with H&E (Section 2.11.2) and blindly scored using the system outlined in section 2.11.4.

Histology Haematoxylin/Eosin Staining

Before commencing with H&E staining, slides were heated to 56° C. for a minimum of 1 hour to aid wax clearance. Slides were then transferred to Xylene (Sigma-Aldrich) for 10 minutes. This was repeated with fresh xylene for a further 10 minutes. Samples were then re-hydrated following immersion in 3 decreasing concentrations of ethanol (100%×2, 90% and 80%) for 5 minutes each. Samples were then transferred to dH₂O for 5 minutes before being immersed in Haemotoxylin (Sigma-Aldrich) for 3 minutes. Samples were then washed under H₂O for 2 minutes before being placed in 1% acid alcohol for no longer than 20 seconds. Samples were washed again under H₂O before being immersed in Eosin Y (Sigma-Aldrich) for 3 minutes and back to washing under H₂O again. Slides were dehydrated through immersion in a series of increasing ethanol concentrations (80%, 90%, 100%) for 5 minutes each. Samples were air dried, mounted with DPX mounting media (BDH) and examined under a light microscope.

Histological Detection of Apoptosis Using Terminal Deoxynucleotidyl Transferase Mediated dUTP Nick End Labeling (TUNEL) Assay

Before commencing with TUNEL assay, slides were heated to 56° C. for a minimum of 1 hour to aid wax clearance. Slides were then transferred to Xylene (Sigma-Aldrich) for 10 minutes. This was repeated with fresh xylene for a further 10 minutes. Samples were then re-hydrated following immersion in 3 decreasing concentrations of ethanol (100%×2, 90% and 80%) for 5 minutes each. Samples were then transferred to dH₂O for 2 minutes before being immersed in boiling antigen unmasking solution (Vector, Peterborough, UK) for 6 minutes. Samples were then washed in PBS for 2 minutes. Tissue sections were circumscribed with ImmEdge™ wax pen (Vector). Once wax was dry. 10 ul of enzyme-label solution (Roche) was added directly onto the tissue. Samples were incubated for 1 hr in a humidified chamber at 37° C. Samples were washed in PBS before 100 ng/ml of DAPI nuclear stain was added to each tissue sample. Slides were incubated at room temperature and protected from light. Samples were air dried, mounted with VectaMount™ aqueous mounting media (Vector) and examined under a fluorescent microscope.

Histological Scoring

Following H&E staining, slides were coded without reference to prior treatment and examined in a blind manner. A semi-quantitative scoring chart was used to assess disease progression in the lungs, liver and GI tract (Tobin et al. 2013). Pathological scoring was carried out as follows:

| Score | Lung | Liver | GI Tract |
|---|---|---|---|
| 0 | Normal | Normal | Normal |
| 1 | Rare scattered areas of | Isolated collections of | Mild necrotic cells with minor |
| | mononuclear cells | mononuclear cells in the | mononuclear cell infiltration |
| | | parenchyma | |
| 2 | Mild and more focused | Endothelialitis present in at | Dispersed but mild villous |
| | areas of mononuclear | least one vessel and distinct | blunting, necrosis and |
| | cell infiltration | increase in mononuclear cell | increased cell infiltration |
| | | infiltration | |
| 3 | Moderate levels of | Endothelialitis present in more | Dispersed and moderate |
| | cellular infiltration and | than one vessel with a further | villous blunting, necrosis with |
| | damage to lung | increase in mononuclear cell | further increased cell |
| | architecture | infiltration | infiltration |
| 4 | Pervasive mononuclear | Endothelialitis present in | Dispersed and severe villous |
| | cell infitration with | virtually all vessels with | blunting, necrotic cells with |
| | pervasive damage to | extensive levels of | pervasive mononuclear cell |
| | lung architecture | mononuclear cell infiltration | infiltration |

Detection of CsA in the Lung, Liver, Spleen & GI Tract of Mice

The lungs, liver, spleen and GI tract were harvested from experimental mice at day 13 and immediately snap frozen and stored at −80° C. Tissues were thawed to room temperature. 500 ul of 2% N-Acetyl-L-Cysteine (Sigma-Aldrich) was added to each sample. Samples were vortexed for 10 minutes before being centrifuged at 13000 rpm for 2 minutes and the liquid phase was collected into a 15 ml tube. The liquid phase (A) and the remaining residue were prepared separately at this point. 500 ul 50% EtOH (Sigma-Aldrich) was added to the remaining residue with 25 ul of internal standard working solution. The tissue in the remaining residue was destroyed using an ultrasonic processor (cycle: 0.5 seconds, max. Amplitude) for 30 seconds. The liquid phase (B) was collected into a 15 ml tube. 500 ul 50% EtOH (Sigma-Aldrich) was added to the remaining residue and vortexed for 1 minute. All remaining liquid was transfered to the 15 ml tube with liquid phase (B). 500 ul 50% EtOH (Sigma-Aldrich) was added to liquid phase (A) with 25 ul of internal standard working solution. 4 ml of diisopropylether (Sigma-Aldrich) was added to liquid phase (A) and (B). (A) and (B) were vortexed vigorously for 5 minutes, centrifuged at 4000 rpm for 2 minutes before being stored at −80° C. for 10 minutes. The organic liquid phase was collected from (A) and (B) and evaporated using a speedy vac (DNA Speedy Vac Concentrator, Thermo Scientific) at 40° C. for 15 minutes. 50 ul of 50% EtOH (Sigma-Aldrich) was added to (A) and (B). (A) and (B) were vortexed for 2 minutes and centrifuged at 4000 rpm for 1 minute. Approx 50 ul were transfered into a conical auto-sampler vial for injection for LC-MS analysis.

Cyclosporin levels in the GI tissues might be higher for Composition A-E of the present invention than for iv administered cyclosporin (Sandimmune).

### Example 5: Survival and Weight Change Data

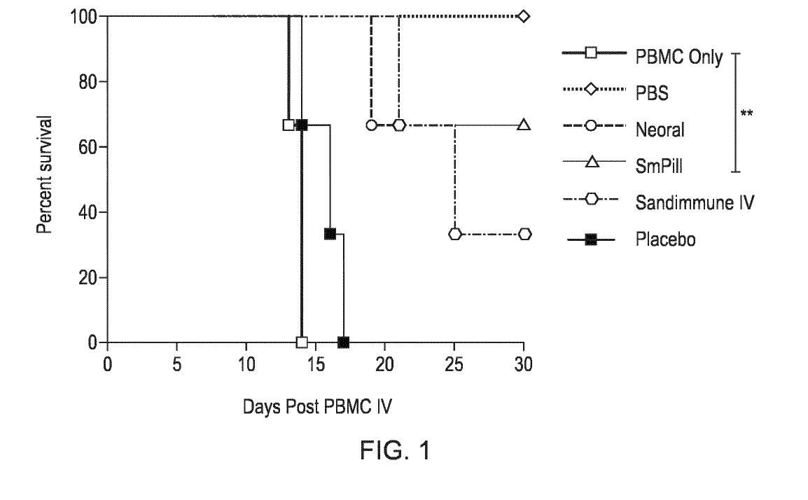

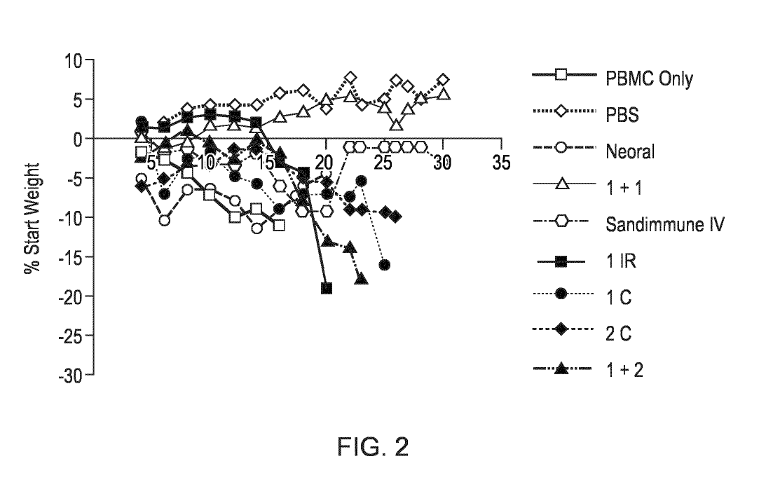

The survival data of each mouse group is shown in FIG. 1 whilst the weight change data is shown in FIG. 2. It is apparent from FIG. 1 that the compositions of the invention provided an improved survival rate compared to the mice not treated with cyclosporin. Certain compositions of the invention provided statistically significant improvements in the survival rate compared to the untreated group. Notably, Neoral did not provide a statistically significant improvement in the survival rate.

A similar trend is observed with the weight change data. Weight loss was lower in the groups administered with cyclosporin compared to the untreated group.

### Example 6: T-Cell Engraftment

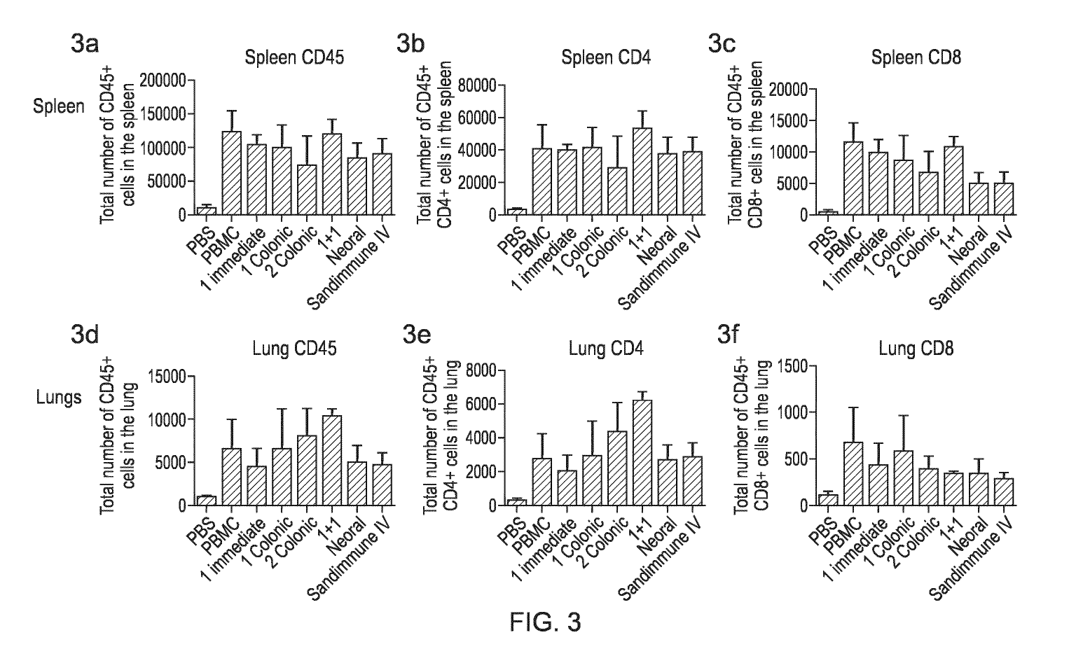

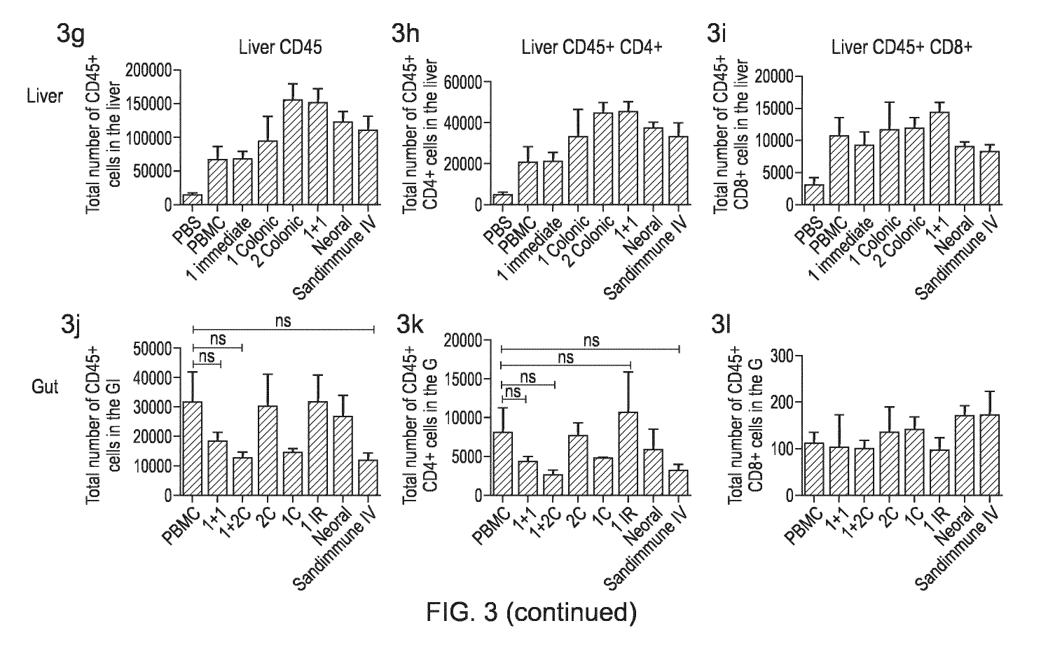

FIGS. 3a-l show data relating to T cell levels in the spleen, lungs, liver and gut of the mice. The total number of CD45+ cells (FIGS. 3a, 3d, 3g and 3j), CD4+ cells (3b, 3e, 3h and 3k) and CD8+ cells (FIGS. 3c, 3f, 3i and 3l) are shown for each group of mice. It is apparent that the total number of cells is not significantly reduced by compositions of the present invention. Engraftment of the donor, infused T cells is not impaired by administration of compositions of the present invention in the spleen, lungs, liver or gut.

### Example 7: Colon Pro-Inflammatory Cytokines

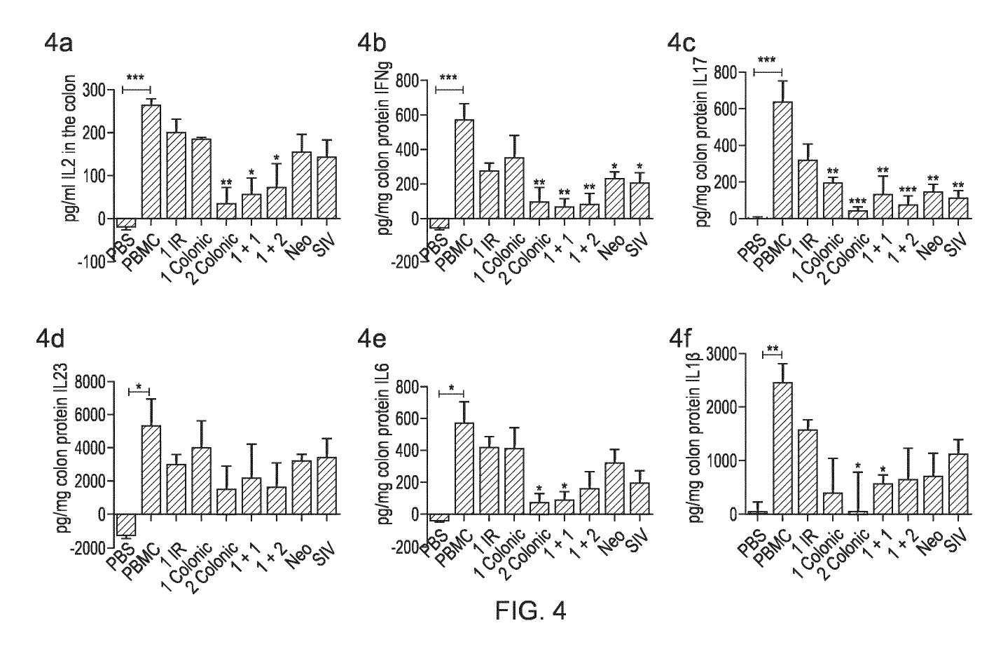

FIGS. 4a-f show the amount of IL2, IFNg, IL17, IL23, IL6 & IL1b, respectively, in the colon of mice from each group. Compositions of the invention significantly reduces the production of certain cytokines related to cytokine release syndrome in the colon of T cell infused mice.

### Example 8: Small Intestine Pro-Inflammatory Cytokines

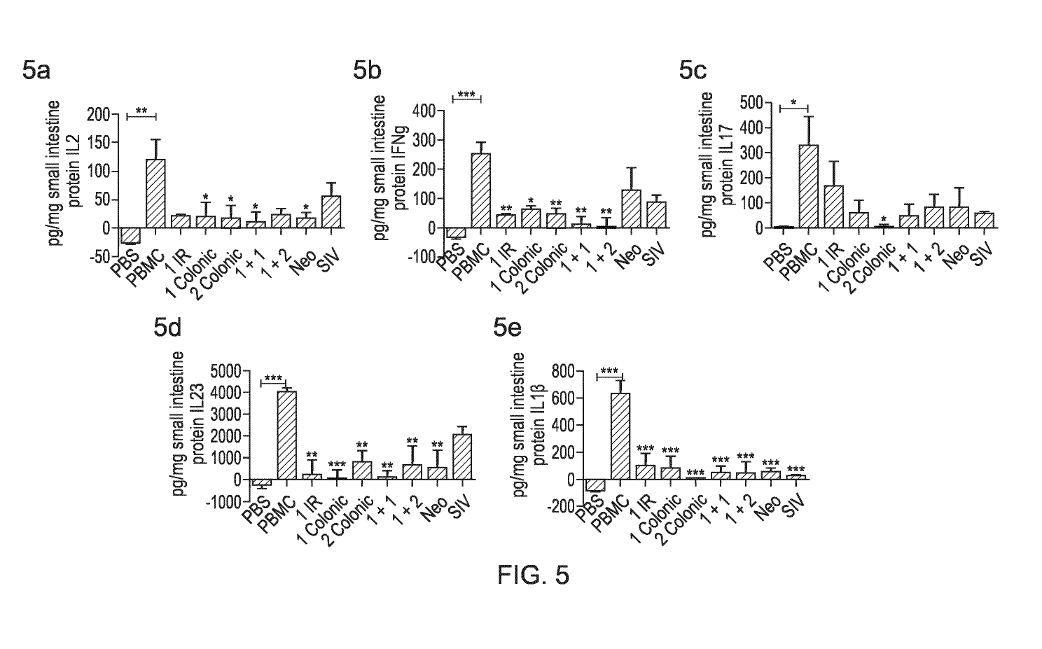

FIGS. 5a-e show the amount of IL2, IFNg, IL17, IL23, & IL1b in the small intestine. As in the colon, and FIGS. 4a-f, compositions of the invention reduce the number of cytokines in the small intestine.

Compare the data of Example 7 and FIGS. 3a-l with that of Example 8 and Example 9 and it is apparent that compositions of the invention do not impair T cell engraftment but do statistically significantly reduce the amount of certain cytokines which play an important role in CRS.

### Example 9: Spleenic Pro-Inflammatory Cytokines

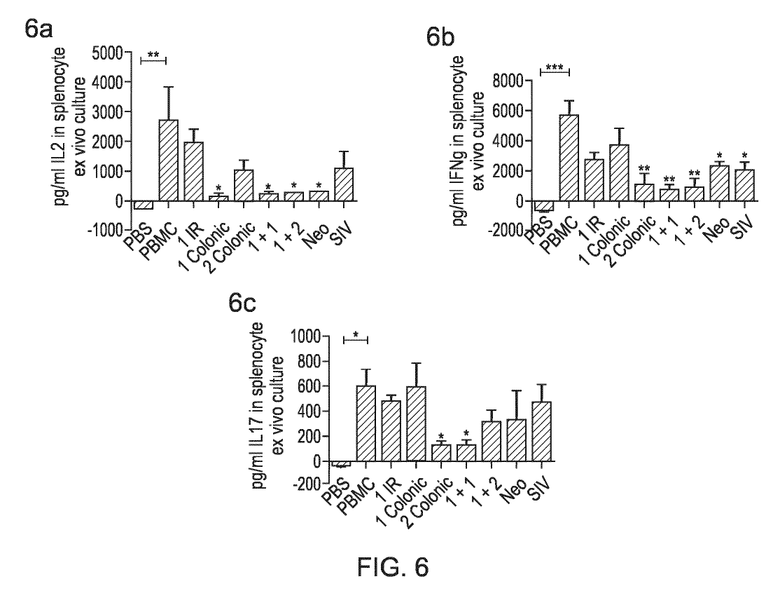

FIGS. 6a-c show that cyclosporin significantly reduces the production of IL-2, IFNg and IL17 in the spleens of mice. In contrast, FIGS. 3a-c show that T cell engraftment is not statistically significantly effected.

### Example 10: Lung Pro-Inflammatory Cytokines

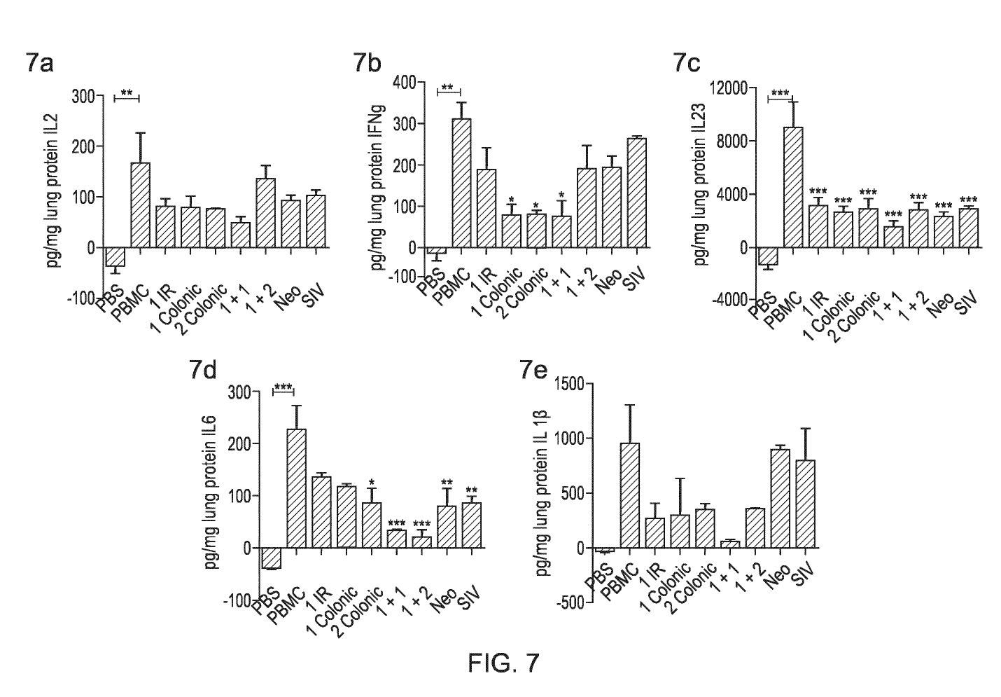

FIGS. 7a-e show that cyclosporin statistically significantly reduces certain pro-inflammatory cytokines in the lung. Again, contrasting the reduction in cytokines against the maintenance of T cell engraftment in shown FIGS. 3d-f shows a beneficial effect of the present invention.

### Example 11: T-Cells in the GIT

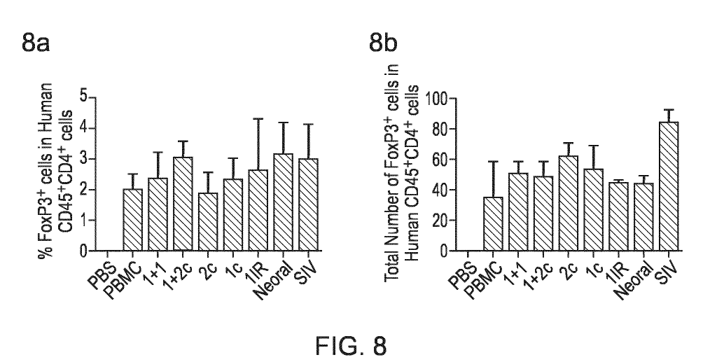

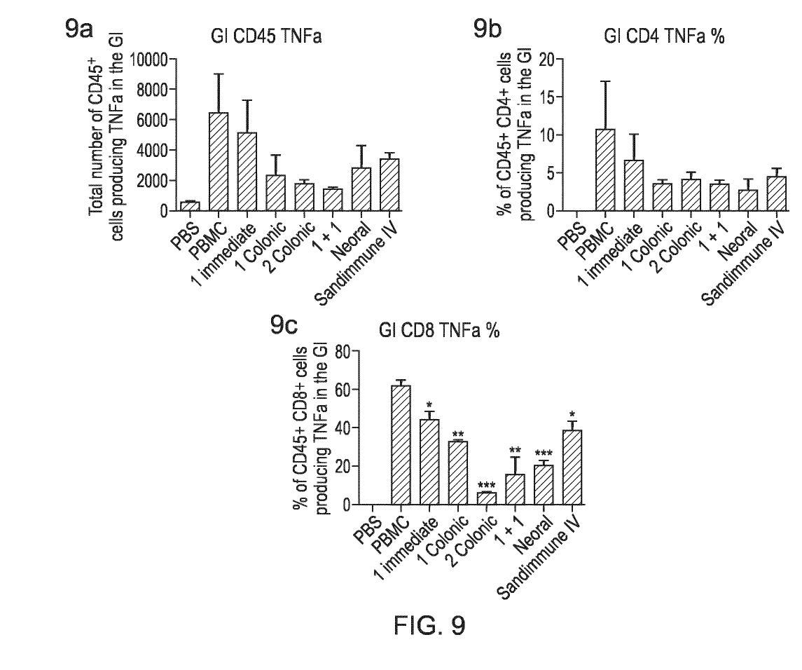

FIGS. 8 and 9 show levels of different types of T cell in the GIT. FIG. 8 shows the levels of FoxP3+, a regulatory T cell, and FIG. 9 shows the levels of TNFα producing T cells. A comparison of FIGS. 8 and 9 shows that the beneficial regulatory T cells, FoxP3+, are not statistically significantly reduced, whereas the cytokine release promoting, TNFα producing T cells are statistically significantly reduced in the GIT. An elevated presence of regulatory T-cells in the GIT is beneficial given that Treg cells control the balance of inflammation by releasing anti-inflammatory agents.

### Example 12: Inflammation and Apoptosis in Intestinal Villi

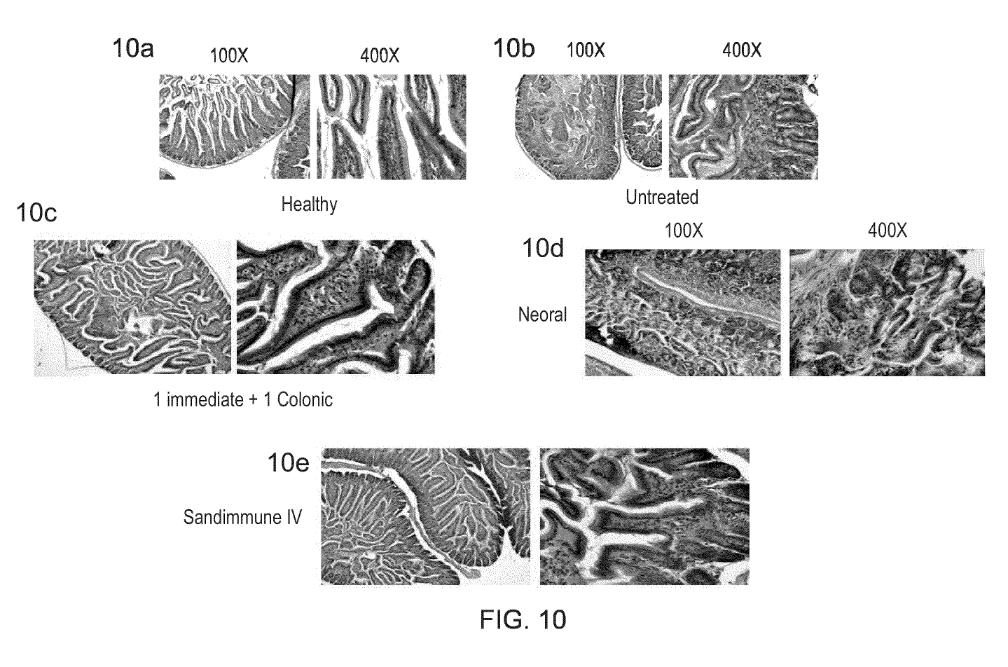

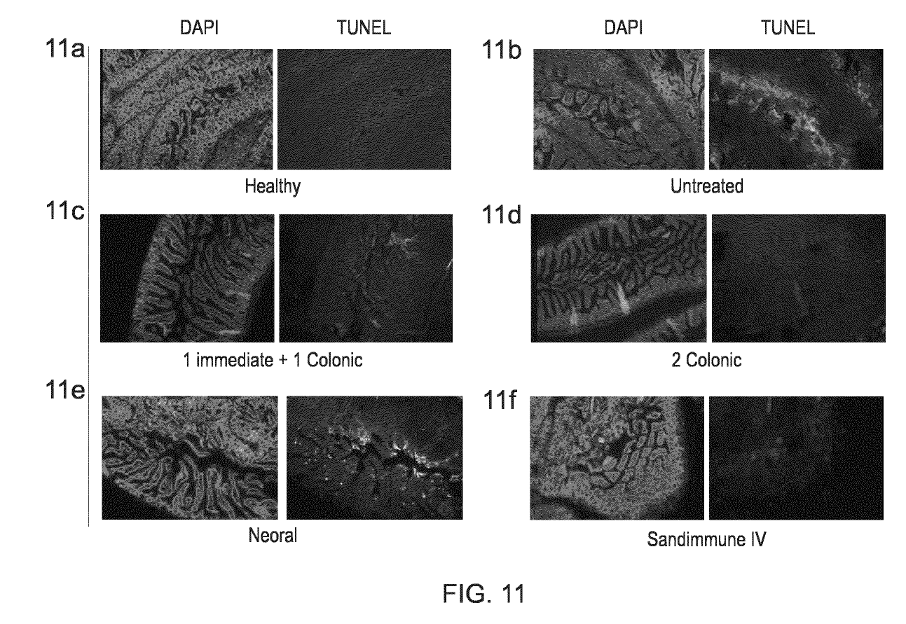

The histological slides in FIGS. 10b and 11b show the inflammation and apoptosis, respectively, of the villi of the small intestine in the untreated mouse group. The untreated mouse group show high levels of inflammation and apoptosis compared to the healthy mouse model. The mouse group administered with the composition of the invention shows relatively minimal inflammation and low (near natural) levels of apoptosis.

The mouse model of Example 4 produces high levels of T effector cells in untreated mice This is evident from FIG. 9 which shows a significant amount of TNFα producing T-cells in the untreated mouse compared to the healthy control mouse. This is equivalent to a NFAT mediated T-cell therapy where the therapy may comprise the administration of T effector cells or where the therapy promotes T effector cells. The data presented in the present application shows that in the presence of high T effector cell levels cytokines throughout the body (in the lung, liver, spleen, small intestine and colon) are reduced compared to untreated, see FIGS. 4 to 7. In contrast, and crucially, the T-cell levels throughout the body are largely, unaffected, see FIG. 3. Furthermore, it appears that T-cell homing into the different body tissues has not been affected. Thus, the maintenance of a NFAT mediated T-cell therapy might be expected based on the data contained herein. Furthermore, the reduction in cytokine levels throughout the body suggests that cytokine release syndrome would be ameliorated. In addition, the percentage of TNF-alpha producing T-cells in the GIT have been reduced in mice administered with a composition of the invention, see FIG. 9. TNF-alpha in the GIT plays a crucial role in GI inflammatory processes.

The above-noted studies, together with available knowledge on the role of NFAT-activation in T cells, leads the inventors to propose a new form of adjunct therapy for cancer therapies mediated by T cells such as CAR-T therapies and T-cell activators such as check-point inhibitors and bi-specific antibodies aimed at combating undesirable effects liable to occur in conjunction with such cancer therapies, more particularly CRS and symptoms associated with gastrointestinal inflammation that result from dysregulated T-cell activity. The above-noted studies lay foundation for such adjunct therapy by co-administration of an inhibitor of NFAT-activation to the GI tract, more particularly for example, oral administration of cyclosporin A formulated for such delivery to the GI, while enabling desired action of T cells for the cancer therapy to continue. As hereinbefore indicated, the ability to hit unwanted T cell activity while maintaining effective T cell-mediated cancer therapy in this manner importantly renders even routine prophylactic use of an inhibitor of NFAT activation such as cyclosporin A plausible for the same purpose. This is seen as a significant contribution to reducing commonly reported undesirable effects which may hamper application of T cell mediated therapies.

1. A method of treating in a subject one or more undesirable effects occurring in conjunction with a therapy mediated by NFAT-activated T cells, wherein said undesirable effects are selected from Cytokine Release Syndrome (CRS) and symptoms associated with gastrointestinal inflammation, the method comprising administering an inhibitor of NFAT activation to the subject, wherein said composition is administered to the gastrointestinal tract whereby said one or more undesirable effects are treated with maintenance of effectiveness of the therapy.

2. The method of claim 1, wherein the therapy is a T cell engaging therapy wherein T cells are activated when directly or indirectly bound to a target disease antigen via a receptor at the surface of the T cells.

3. The method of claim 1, wherein the therapy is an immune checkpoint blockade therapy.

4. The method of claim 3 wherein said therapy is an immune checkpoint blockade therapy employing one or more antibodies targeting a T cell inhibitory pathway.

5. The method of claim 4 wherein said checkpoint blockade therapy employs an anti-CTLA-4 antibody and/or an antibody which targets ligand binding to the PD1 receptor.

6. The method of claim 2 wherein said T cell engaging therapy is any of a bispecific T cell engager or a chimeric antigen receptor therapy.

7. The method of claim 6, wherein the bispecific T cell engager is a bispecific antibody.

8. The method of claim 6, wherein the chimeric antigen receptor therapy is a CAR-T cell therapy.

9. The method of claim 8, wherein the CAR therapy is allogenic.

10. The method of claim 8, wherein the CAR therapy is autologous.

11. The method of claim 1, wherein said therapy is mediated by NFAT-activated autologous T cells.

12. The method of claim 1 wherein the undesirable effect is CRS.

13. The method of claim 1, wherein the therapy is autologous CAR-T therapy and administration of said composition to the gastrointestinal tract reduces or prevents CRS while maintaining effectiveness of said therapy.

14. The method of claim 1, wherein the therapy is allogenic CAR-T therapy and administration of said composition to the gastrointestinal tract reduces or prevents CRS while maintaining effectiveness of said therapy.

15. The method of claim 12 wherein the CAR-T therapy is for the treatment of a cancer.

16. A method of claim 1 wherein the therapy comprises a pre-conditioning regime to deplete autologous T cells of the patient.

17. The method of claim 1 wherein the therapy comprises two or more therapies selected from a bispecific T cell engager, a chimeric antigen receptor therapy and a checkpoint blockade therapy, for example wherein the therapy comprises a checkpoint blockade therapy and one or more therapy selected from a bispecific T cell engager and a chimeric antigen receptor therapy.

18. (canceled)

19. The method of claim 1, wherein the one or more undesirable effects are symptoms associated with gastrointestinal inflammation, and wherein the symptoms of gastrointestinal inflammation are associated with an inflammatory bowel disease, optionally a condition selected from: irritable bowel disease, Crohn's disease, ulcerative colitis, celiac disease, gastroenteritis, duodenitis, jejunitis, ileitis, peptic ulcer, pouchitis, Curling's ulcer, appendicitis, colitis, pseudomembraneous colitis, diverticulosis, diverticulitis, collagenous colitis, systemic inflammation optionally emanating from the GIT, colorectal carcinoma and adenocarcinoma.

20. The method of claim 19, wherein said symptoms of inflammatory bowel disease are associated with ulcerative colitis or Crohn's disease.

21-22. (canceled)

23. The method of claim 1 wherein said inhibitor is selected from: cyclosporin, cyclosporin derivatives, tacrolimus derivatives, pyrazoles, pyrazole derivatives, phosphatase inhibitors, S1P receptor modulators, toxins, paracetamol metabolites, fungal phenolic compounds, coronary vasodilators, phenolic adeide, flavanols, thiazole derivatives, pyrazolopyrimidine derivatives, benzothiophene derivatives, rocaglamide derivatives, diaryl triazoles, barbiturates, antipsychotics (penothiazines), serotonin antagonists, salicylic acid derivatives, phenolic compounds derived from propolis or pomegranate, imidazole derivatives, pyridinium derivatives, furanocumarins, alkaloids, triterpenoids, terpenoids, oligonucleotides, or peptides.

24. (canceled)

25. The method of claim 1, wherein the composition is a solid composition comprising an enteric coating.

26. The method of claim 1, further comprising a hydrogel forming polymer matrix, a surfactant and an oil phase.

27. The method of claim 26, wherein the oil phase is dispersed in the hydrogel forming polymer matrix.

28. The method of claim 26 wherein the oil phase comprises a solution of the NFAT inhibitor.

29-42. (canceled)

43. The method of claim 26, wherein the surfactant comprises a long chain fatty acid mono- or di-glyceride or a combination thereof.

44. The method of claim 26, wherein the surfactant comprises a surfactant selected from:
glyceryl monocaprate, glyceryl dicaprate, glyceryl monocaprylate, glyceryl dicaprylate, glyceryl caprate, glyceryl monocaprylate/caprate, glyceryl caprylate/caprate glyceryl dicaprylate/caprate, glyceryl monooleate/dioleate, glyceryl monooleate, glyceryl dioleate, glyceryl monostearate, glyceryl distearate, glyceryl monopalmitostearate, glyceryl dipalmitostearate, glyceryl monobehenate, glyceryl dibehenate, glyceryl monolinoleate, glycerol monolinoleate, glyceryl dilinoleate, polyglyceryl dioleate, propylene glycol monoheptanoate, polyglycerol dioleate, and a combination thereof.

45-47. (canceled)

48. The method of claim 26, wherein the composition further comprises a second surfactant, optionally wherein the second surfactant is an anionic surfactant, optionally selected from alkyl sulphates, carboxylates or phospholipids, or a non-ionic surfactant, optionally selected from sorbitan-based surfactants, PEG-fatty acids, or glyceryl fatty acids, or poloxamers, or a combination thereof.

49-62. (canceled)

63. The method of claim 1, wherein the composition further comprises at least one coating.

64. The method of claim 63 wherein the at least one coating is adapted to release the NFAT inhibitor, for example cyclosporin, in at least the colon.

65-80. (canceled)

81. The method of claim 1, wherein the composition comprises a colloidal composition comprising:
a continuous phase comprising:
a hydrogel forming polymer matrix comprising gelatin in an amount of 300 to 700 mg/g;
a disperse phase comprising:
cyclosporin in an amount of up to 200 mg/g; and
a medium chain tri-glyceride in an amount of 20 to 200 mg/g;
and the composition further comprising:
solvent in an amount of 100 to 250 mg/g;
surfactant having an HLB of up to 8 in an amount of 80 to 200 mg/g; and
anionic surfactant in an amount of up to 50 mg/g.

82-88. (canceled)
